(19) United States
(12) Patent Application Publication
(10) Pub. No.: US 2017/0037425 A1
(43) Pub. Date: Feb. 9, 2017

(54) METHOD FOR IMPROVING PLANT TRAIT

(71) Applicant: SHANGHAI INSTITUTES FOR BIOLOGICAL SCIENCES, CHINESE ACADEMY OF SCIENCES, Shanghai (CN)

(72) Inventors: Weiming CAI, Shanghai (CN); Weining SUN, Shanghai (CN); Genyun CHEN, Shanghai (CN); Xinguang ZHU, Shanghai (CN); Hualing MI, Shanghai (CN); Haiying CHEN, Shanghai (CN)

(73) Assignee: SHANGHAI INSTITUTES FOR BIOLOGICAL SCIENCES, CHINESE ACADEMY OF SCIENCES, Shanghai (CN)

(21) Appl. No.: 14/370,337

(22) PCT Filed: Dec. 31, 2012
(86) PCT No.: PCT/CN2012/087995

§ 371 Date: Jul. 2, 2014

(30) Foreign Application Priority Data
Jan. 5, 2012 (CN) 201210002157.9

(51) Int. Cl. C12N 15/82; C07K 14/435

(52) U.S. Cl. CPC C12N 15/8269; C12N 15/8209; C07K 14/43504

(57) ABSTRACT

Provided is a method for plant improvement with the aim of increasing the light use efficiency of a plant. By expressing a light energy absorption and transduction (LEAT) protein in the plant, and by utilizing light energy absorbed to interact with a related methyl-quinone derivative in the plant body, such as plastoquinone, to catalyze water splitting and to release oxygen, the present invention increases the light use efficiency of the plant. The method effectively extends the utilization of light energy by the plant, thus increasing photosynthesis efficiency and yield.

### TECHNICAL FIELD

The invention belongs to biological technology, more particular to methods for improving plant traits.

### BACKGROUND ART

Photosynthesis is the most important chemical reaction on earth, by which plant can transfer light into stable chemical energy that can be used by humans and oxygen as well as food necessary for the survival of humans. However, with regard to the light energy utilization efficiency of a plant, which is calculated for the whole growth season, the crops in the field could utilize less than 1% of the light energy on the ground surface which the crops receive during the growth season. The utilization of light by a plant is mainly dependent on the following aspects: first, capturing light by a plant. For a higher plant, the efficiency of light energy capturing by leaves can reach more than 80%; wherein chlorophyll a and b mainly absorb the red and blue light portion for photosynthesis, carotenoids as accessory photosynthesis pigments mainly absorb blue and near ultraviolet light; and green and yellow light and the like are partly reflected and transmitted so that these lights may not be utilized adequately. Second, the excess UV portion (260-400 nm) of sunlight is harmful to plant cells, for example, excess UV light leads to damage in membrane system of plant cells and the function of a plant photosynthetic apparatus. Third, the light energy utilization efficiency is dependent on the transformation efficiency of the light energy captured by a plant, which is light energy transformation efficiency. The light energy transformation efficiency is not only dependent on the photoreaction on the thylakoid membranes of chloroplasts, but is affected by the dark reaction and photorespiration in the chloroplast stroma as well. Therefore, all the factors related to these aspects will affect the light utilization efficiency of plant leaves, for example, the protection mechanism or the light repair ability of a plant photosystem; intracellular CO₂ concentration in the leaf of a higher plant, which is controlled by stomata opening and closing, and the stomata opening and closing is driven by the ability to capture available photosynthetic energy by the chloroplast in stomata guard cells. In the background of the global population growth, food crisis and energy crisis, it is of great importance to find new means to increase the light utilization efficiency of a plant.

Under general conditions, sunlight and the raw materials such as water and carbon dioxide for photosynthesis are not absent, however, for internal and external reasons, the utilization efficiency of solar energy by the plant is very low, it is estimated that the annual mean conversion rate of the plant photosynthesis in temperate zone is about 0.5-2.5% of the whole solar radiation energy, the mean conversion rate of the whole biosphere is 3-5%. In ideal circumstance and condition, the highest photosynthesis efficiency can be up to 8-15%. Therefore, the increase of the light energy utilization efficiency is a great breakthrough to further increase energy biological yield.

For many years, various attempts have been made to increase photosynthesis efficiency of plants, the main strategies include reduction of the loss in photorespiration, increasing the ratio between plant Rubisco carboxylation and oxidation, and transforming a C3 plant into a C4 plant, etc. All these strategies focus on one certain aspect affecting photosynthesis efficiency of plants. At present, there is no approach confirmed to effectively increase light energy utilization efficiency of a plant. As the photosynthetic apparatus is very conservative among various plants, once an approach to increase light energy utilization efficiency is fully verified, its huge potential can be applied in different plants. Therefore, there is a need to investigate and develop a novel method for increasing light utilization efficiency of a plant to breed plants with higher light energy utilization efficiency, thereby increasing the yield of plants.

### SUMMARY OF INVENTION

The object of the invention is to provide a method for improving plant traits.

In the first aspect, the invention provides a method for improving plant traits, comprising the steps of: 1) transforming one or more polynucleotides encoding Light Energy Absorption and Transduction protein (LEAT protein) into plants; 2) selecting the plants with improved traits compared with a control plant from the transformed plants; said light energy absorption and transduction protein is a protein which can utilize light energy to catalyze water hydrolysis and 2,3,5-trimethyl-1,4-benzoquinone reduction.

In a preferred embodiment, said polynucleotide encoding the light energy absorption and transduction protein is selected from the group consisting of (a) a polynucleotide encoding a fluorescent protein or its mutant proteins with one or more (such as 1 to 30, preferably 1 to 20, more preferably 1 to 10, even more preferably 1 to 5) amino acid site mutations which are changed in fluorescence intensity and fluorescence emission spectra but can still utilize light energy to catalyze water hydrolysis and 2,3,5-trimethyl-1,4-benzoquinone reduction; or (b) a polynucleotide encoding a non-fluorescent chromoprotein or its mutant proteins with one or more (such as 1 to 30, preferably 1 to 20, more preferably 1 to 10, even more preferably 1 to 5) amino acid site mutations which can still utilize light energy to catalyze water hydrolysis and 2,3,5-trimethyl-1,4-benzoquinone reduction.

In another preferred embodiment, said polynucleotide encoding a fluorescent protein or its mutant proteins with one or more (such as 1 to 30, preferably 1 to 20, more preferably 1 to 10, even more preferably 1 to 5) amino acid site mutations which are changed in fluorescence intensity and fluorescence emission spectra but can still utilize light energy to catalyze water hydrolysis and 2,3,5-trimethyl-1,4-benzoquinone reduction is selected from the groups consisting of:

(a) a polynucleotide encoding the protein with the amino acid sequence as set forth in SEQ ID NO: 4;

(b) a polynucleotide encoding the protein with the amino acid sequence as set forth in SEQ ID NO: 10;

(c) a polynucleotide encoding the protein (efasCFP) with the amino acid sequence as set forth in SEQ ID NO: 36;

(d) a polynucleotide encoding the protein with the amino acid sequence as set forth in SEQ ID NO: 6;

(e) a polynucleotide encoding the protein with the amino acid sequence as set forth in SEQ ID NO: 28;

(f) a polynucleotide encoding the protein (scubGFP) with the amino acid sequence as set forth in SEQ ID NO: 40;

(g) a polynucleotide encoding the protein (rmueGFP) with the amino acid sequence as set forth in SEQ ID NO: 44;

(h) a polynucleotide encoding the protein (cpGFP) with the amino acid sequence as set forth in SEQ ID NO: 52;

(i) a polynucleotide encoding the protein (YFP) with the amino acid sequence as set forth in SEQ ID NO: 8;

(j) a polynucleotide encoding the protein (YFPmu2) with the amino acid sequence as set forth in SEQ ID NO: 12;

(k) a polynucleotide encoding the protein (YFPmu4) with the amino acid sequence as set forth in SEQ ID NO: 14;

(l) a polynucleotide encoding the protein (YFPmu7) with the amino acid sequence as set forth in SEQ ID NO: 16;

(m) a polynucleotide encoding the protein (YFP^{L232H}) with the amino acid sequence as set forth in SEQ ID NO: 18;

(n) a polynucleotide encoding the protein (YFP^{L232Q}) with the amino acid sequence as set forth in SEQ ID NO: 20;

(o) a polynucleotide encoding the protein (phiYFP) with the amino acid sequence as set forth in SEQ ID NO: 50;

(p) a polynucleotide encoding the protein (mCherry) with the amino acid sequence as set forth in SEQ ID NO: 2;

(q) a polynucleotide encoding the protein (mCherrymu3) with the amino acid sequence as set forth in SEQ ID NO: 22;

(r) a polynucleotide encoding the protein (mCherrymu4) with the amino acid sequence as set forth in SEQ ID NO: 24;

(s) a polynucleotide encoding the protein (mCherrymu5) with the amino acid sequence as set forth in SEQ ID NO: 26;

(t) a polynucleotide encoding the protein (eqFP611) with the amino acid sequence as set forth in SEQ ID NO: 30;

(u) a polynucleotide encoding the protein (eforCP/RFP) with the amino acid sequence as set forth in SEQ ID NO: 34;

(v) a polynucleotide encoding the protein (rfloRFP) with the amino acid sequence as set forth in SEQ ID NO: 42;

(w) a polynucleotide encoding the protein (ceriantRFP) with the amino acid sequence as set forth in SEQ ID NO: 46;

(x) a polynucleotide encoding the protein (hcriCP) with the amino acid sequence as set forth in SEQ ID NO: 32;

(y) a polynucleotide encoding the protein (anm2CP) with the amino acid sequence as set forth in SEQ ID NO: 48;

(z) a polynucleotide encoding the protein (YFP₁₋₂₃₁) with the amino acid sequence as set forth in SEQ ID NO: 62;

(aa) a polynucleotide encoding the protein (GFP₁₋₂₃₁) with the amino acid sequence as set forth in SEQ ID NO: 64;

(ab) a polynucleotide encoding a protein formed by one or more (such as 1 to 30, preferably 1 to 20, more preferably 1 to 10, even more preferably 1 to 5) amino acid residue substitution, deletion or addition in any amino acid sequence in (a) to (aa) and having the ability to utilize light energy to catalyze water hydrolysis and 2,3,5-trimethyl-1,4-benzoquinone reduction;

(ac) a polynucleotide encoding the protein which has more than 70% (more preferably more than 80%, more preferably more than 90%, more preferably more than 95%, more preferably more than 98%, more preferably more than 99%) sequence identity to the protein with any amino sequence in (a) to (aa) and having the ability to utilize light energy to catalyze water hydrolysis and 2,3,5-trimethyl-1,4-benzoquinone reduction;

or

(ad) a polynucleotide complementary to any polynucleotide in (a) to (ac) above.

In another preferred embodiment, said polynucleotide encoding a fluorescent protein or its mutant proteins with one or more (such as 1 to 30, preferably 1 to 20, more preferably 1 to 10, even more preferably 1 to 5) amino acid site mutations which are changed in fluorescence intensity and fluorescence emission spectra but can still utilize light energy to catalyze water hydrolysis and 2,3,5-trimethyl-1,4-benzoquinone reduction is selected from the groups consisting of:

(a) a polynucleotide with the nucleotide sequence as set forth in SEQ ID NO: 3;

(b) a polynucleotide with the nucleotide sequence as set forth in SEQ ID NO: 9;

(c) a polynucleotide with the nucleotide sequence as set forth in SEQ ID NO: 35;

(d) a polynucleotide with the nucleotide sequence as set forth in SEQ ID NO: 5;

(e) a polynucleotide with the nucleotide sequence as set forth in SEQ ID NO: 27;

(f) a polynucleotide with the nucleotide sequence as set forth in SEQ ID NO: 39;

(g) a polynucleotide with the nucleotide sequence as set forth in SEQ ID NO: 43;

(h) a polynucleotide with the nucleotide sequence as set forth in SEQ ID NO: 51;

(i) a polynucleotide with the nucleotide sequence as set forth in SEQ ID NO: 7;

(j) a polynucleotide with the nucleotide sequence as set forth in SEQ ID NO: 11;

(k) a polynucleotide with the nucleotide sequence as set forth in SEQ ID NO: 13;

(l) a polynucleotide with the nucleotide sequence as set forth in SEQ ID NO: 15;

(m) a polynucleotide with the nucleotide sequence as set forth in SEQ ID NO: 17;

(n) a polynucleotide with the nucleotide sequence as set forth in SEQ ID NO: 19;

(o) a polynucleotide with the nucleotide sequence as set forth in SEQ ID NO: 49;

(p) a polynucleotide with the nucleotide sequence as set forth in SEQ ID NO: 1;

(q) a polynucleotide with the nucleotide sequence as set forth in SEQ ID NO: 21;

(r) a polynucleotide with the nucleotide sequence as set forth in SEQ ID NO: 23;

(s) a polynucleotide with the nucleotide sequence as set forth in SEQ ID NO: 25;

(t) a polynucleotide with the nucleotide sequence as set forth in SEQ ID NO: 29;

(u) a polynucleotide with the nucleotide sequence as set forth in SEQ ID NO: 33;

(v) a polynucleotide with the nucleotide sequence as set forth in SEQ ID NO: 41;

(w) a polynucleotide with the nucleotide sequence as set forth in SEQ ID NO: 45;

(x) a polynucleotide with the nucleotide sequence as set forth in SEQ ID NO: 31;

(v) a polynucleotide with the nucleotide sequence as set forth in SEQ ID NO: 47;

(z) a polynucleotide with the nucleotide sequence as set forth in SEQ ID NO: 61;

(aa) a polynucleotide with the nucleotide sequence as set forth in SEQ ID NO: 63; or

(ab) a polynucleotide complementary to any of the polynucleotide in (a) to (aa).

In another embodiment, said polynucleotide encoding a non-fluorescent chromoprotein or its mutant proteins with one or more (such as 1 to 30, preferably 1 to 20, more preferably 1 to 10, even more preferably 1 to 5) amino acid site mutations which can still utilize light energy to catalyze water hydrolysis and 2,3,5-trimethyl-1,4-benzoquinone reduction is selected from the group consisting of:

(a) a protein (spisCP) with the amino acid sequence as set forth in SEQ ID NO: 38;

(b) a protein formed by one or more (such as 1 to 30, preferably 1 to 20, more preferably 1 to 10, even more preferably 1 to 5) amino acid residue substitution, deletion or addition in the amino acid sequence as set forth in (a) and having the ability to utilize light energy to catalyze water hydrolysis and 2,3,5-trimethyl-1,4-benzoquinone reduction;

(c) a protein having more than 70% (more preferably more than 80%), more preferably more than 90%, more preferably more than 95%, more preferably more than 98%, more preferably more than 99%) sequence identity to the protein with the amino sequence as set forth in (a) and having the ability to utilize light energy to catalyze water hydrolysis and 2,3,5-trimethyl-1,4-benzoquinone reduction; or

(d) a polynucleotide complementary to any of the polynucleotide in (a) to (c).

In another preferred embodiment, said polynucleotide encoding a non-fluorescent chromoprotein or its mutant proteins with one or more (such as 1 to 30, preferably 1 to 20, more preferably 1 to 10, even more preferably 1 to 5) amino acid site mutations which can still utilize light energy to catalyze water hydrolysis and 2,3,5-trimethyl-1,4-benzoquinone reduction is selected from the group consisting of:

(a) a polynucleotide with the nucleotide sequence as set forth in SEQ ID NO: 37; or

(b) a polynucleotide complementary to the polynucleotide of (a).

In another preferred embodiment, said polynucleotide encoding light energy absorption and transduction protein is further selected from the group consisting of: a polynucleotide encoding the protein selected from the following Genbank accession numbers: API68421, AY646070, AY646072, AY646071, AF168424, AF168420, AY182022, AY182023, DQ206381, DQ206392, DQ206382, AY181556, AY679113, EU498721, AY182017, AY646069, AY646066, AY151052, EU498722, AY647156, AY508123, AY508124, AY508125, AF435432, AY646067, AY485334, AY485335, AY646068, AF545827, AF545830, AY037776, AB180726, DQ206383, DQ206395, DQ206396, DQ206385, EU498723, AB193294, AY182020, AY182021, AB107915, DQ206389, P42212, AY268073, AY181553, AY181554, AY181555, DQ206393, AY155344, AY037766, AY679112, AF401282, EU498724, AY268076, AY268074, AY268075, AY268071, AY268072, DQ206391, AY015995, AY182014, AF372525, EU498725, DQ206390, AF168422, AY646073, AY296063, AF272711, AB128820, DQ206379, DQ206380, AF168419, AY059642, EF186664, AF420591, DQ206387, AY182019, AY765217, AB085641, AY181552, DQ206386, AY182013, AY646064, AY485333, AF168423, AY646077, AY646076, AY646075, EF587182, AF363775, AF383155, DQ206394, DQ206376, AF38315, AF363776, AY461714, AB209967, DQ206377, DQ206378; or the mutant proteins thereof with one or more amino acid site mutations which are changed in fluorescence intensity and fluorescence emission spectra but can still utilize light energy to catalyze water hydrolysis and 2,3,5-trimethyl-1,4-benzoquinone reduction.

In another preferred embodiment, the method for transforming the polynucleotide into plants comprises: transforming the expression cassette containing the nucleotide encoding light energy absorption and transduction protein into plants, thereby expressing said nucleotide in the plants.

Preferably, the expression cassette comprises at least one (one or more) polynucleotide encoding light energy absorption and transduction protein; said polynucleotide encoding light energy absorption and transduction protein is operable linked to expression regulatory elements.

In another preferred embodiment, said method comprises: (1) providing Agrobacterium tumefaciens carrying the expression vector which contains the expression cassette; (2) contacting the tissue or organ of the plant with the Agrobacterium tumefaciens in (1), thereby transforming the expression cassette into the plant cell, tissue or organ.

In another preferred embodiment, said improved plant trait is one or more select from the group consisting of: increasing the biomass of a plant; increasing the yield of a plant; promoting the growth of a plant; increasing the size of seed or panicle of a plant; increasing the number of seeds, tillers or panicles of a plant; increasing seed size; increasing seed weight; increasing total content of protein of a plant; increasing light utilization efficiency of a plant; increasing photochemical efficiency of PSI or PSII of a plant; increasing plant photosynthetic electron transfer efficiency; increasing CO₂ assimilation ability of a plant; increasing net photosynthetic rate of a plant; increasing the light protection ability of a plant; increasing the content of accessory photosynthetic pigment (including carotenoids); and increasing the photosynthetic O₂ evolution rate of a plant.

Preferably, the improved plant trait is an improved plant trait associated with the yield of a plant, one or more traits selected from the group consisting of: increasing the biomass of a plant; increasing the yield of a plant; increasing the size of seed or panicle; increasing the number of seeds of a plant, tillers or panicles; increasing seed size; and increasing seed weight.

In another preferred embodiment, the plants are gymnosperms, monocotyledonous or dicotyledonous plants.

In another preferred embodiment, the plants comprise: Salicaceae, Moraceae, Myrtaceae, Lycopodiaceae, Selaginellaceae, Ginkgoaceae, Pinaceae, Cycadaceae, Araceae, Ranunculaceae, Platanaceae, Ulmaceae, Juglandaceae, Betulaceae, Actinidiaceae, Malvaceae, Sterculiaceae, Tiliaceae, Tamaricaceae, Rosaceae, Crassulaceae, Caesalpinaceae, Fabaceae, Punicaceae, Nyssaceae, Cornaceae, Alangiaceae, Celastraceae, Aquifoliaceae, Buxaceae, Euphorbiaceae, Pandaceae, Rhamnaceae, Vitaceae, Anacardiaceae, Burseraceae, Campamdaceae, Rhizophoraceae, Santalaceae, Oleaceae, Scrophulariaceae, Gramineae, Pandanaceae, Sparganiaceae, Aponogetonaceae, Potamogetonaceae, Najadaceae, Scheuchzeriaceae, Alismataceae, Butomaceae, Hydrocharitaceae, Triuridaceae, Cyperaceae, Palmae (Arecaceae), Araceae, Lemnaceae, Flagellariaceae, Restionaceae, Centrolepidaceae, Xyridaceae, Eriocaulaceae, Bromeliaceae, Commelinaceae, Pontederiaceae, Philydraceae, Juncaceae, Stemonaceae, Liliaceae, Amaryllidaceae, Taccaceae, Dioscoreaceae, Iridaceae, Musaceae, Zingiheraceae, Annaceae, Marantaceae, Burmanniaceae, Chenopodiaceae or Orchidaceae.

In another preferred embodiment, the plants comprise, but not limited to: wheat, rice, barley, corn, sorghum, oat, rye, cane, Brassica, Chinese cabbage, cotton, soybean, alfalfa, tobacco, tomato, capsicum, pumpkin, watermelon, cucumber, apple, peach, plum, Malus spectabilis, sugar beet, sunflower, lettuce, asparagus lettuce, Artemisia carvifolia, Helianthus tuberosus, stevia, poplar, willow, eucalyptus, lilac, rubber tree, cassava, castor, peanut, pea, astragalus, tobacco, tomato, capsicum, etc.

In another aspect, the invention provides the use of light energy absorption and transduction protein or the polynucleotide encoding thereof for improving a plant trait.

In a preferred embodiment, said light energy absorption and transduction protein is selected from a fluorescent protein or non-fluorescent chromoprotein.

In another aspect, the invention provides separated light energy absorption and transduction proteins, which comprises:

Protein (mCherrymu3) with the amino acid sequence as set forth in SEQ ID NO: 22;

Protein (mCherrymu4) with the amino acid sequence as set forth in SEQ ID NO: 24;

Protein (mCherrymu5) with the amino acid sequence as set forth in SEQ ID NO: 26;

Protein (YFPmu2) with the amino acid sequence as set forth in SEQ ID NO: 12;

Protein (YFPmu4) with the amino acid sequence as set forth in SEQ ID NO: 14;

Protein (YFPmu7) with the amino acid sequence as set forth in SEQ ID NO: 16;

Protein (YFP_{L232H}) with the amino acid sequence as set forth in SEQ ID NO: 18;

Protein (YFP_{L232Q}) with the amino acid sequence as set forth in SEQ ID NO: 20;

Protein (YFP₁₋₂₃₁) with the amino acid sequence as set forth in SEQ ID NO: 62; or

Protein (GFP₁₋₂₃₁) with the amino acid sequence as set forth in SEQ ID NO: 64.

In another aspect, the invention provides a separated polynucleotide encoding any of the said light energy absorption and transduction protein.

In a preferred embodiment, said polynucleotide is selected from:

Polynucleotide (mCherrymu3) with the nucleotide sequence as set forth in SEQ ID NO: 21;

Polynucleotide (mCherrymu4) with the nucleotide sequence as set forth in SEQ ID NO: 23;

Polynucleotide (mCherrymu5) with the nucleotide sequence as set forth in SEQ ID NO: 25;

Polynucleotide (YFPmu2) with the nucleotide sequence as set forth in SEQ ID NO: 11;

Polynucleotide (YFPmu4) with the nucleotide sequence as set forth in SEQ ID NO: 13;

Polynucleotide (YFPmu7) with the nucleotide sequence as set forth in SEQ ID NO: 15;

Polynucleotide (YFP_{L232H}) with the nucleotide sequence as set forth in SEQ ID NO: 17;

Polynucleotide (YFP_{L232Q}) with the nucleotide sequence as set forth in SEQ ID NO: 19;

Polynucleotide (YFP₁₋₂₃₁) with the nucleotide sequence as set forth in SEQ ID NO: 61; or

Polynucleotide (GFP₁₋₂₃₁) with the nucleotide sequence as set forth in SEQ ID NO: 63.

In another aspect, the invention provides a recombinant expression vector containing said polynucleotide.

In another aspect, the invention provides a genetic engineered cell which contains said recombinant expression vector, or the genome of the cell is integrated with said polynucleotide.

In another preferred embodiment, the genetic engineered cell is non-propagation material and non-generative cell.

In another aspect, the invention provides an improved plant, the genome of which contains the expression cassette of light energy absorption and transduction protein. Preferably, said improved plant is a transgenic plant which is obtained by any method above.

Other aspects of the invention are apparent to those skilled in the art based on the disclosure herein.

### BRIEF DESCRIPTION OF THE DRAWINGS

FIG. 1. The effects of light on NAD⁺ or NAPD⁺ reduction by YFP. Figure shows the changes in absorption at 340 nm of NADH and NADPH before and after the light was switched on.

FIG. 2. Comparison of the catalytic activities of YFP, CFP, BFP and GFP on 2,3,5-trimethyl-1,4-benzoquinone (TMBQ) reduction under light. 50 mM phosphate buffer (pH 6.5), TMBQ, 400 μM, concentrations of fluorescent proteins are 25 nM. The reduction rates were measured with the corresponding excitation wavelength of each fluorescent at 1-2 μmol m⁻²s⁻¹.

FIG. 3. Light intensity dependent curve of water hydrolysis and O₂ evolution catalyzed by YFP and GFP in the presence of TMBQ. TMBQ: 2,3,5-trimethyl-1,4-benzoquinone. The reaction rate was represented as the number of the O₂ molecules converted by each protein molecule per minute. Reaction system: 50 mM phosphate buffer (pH6.5), YFP: 10 nM (final concentration, same for the following); GFP: 1 μM; TMBQ: 400 μM; light intensity: 0.6-1 μmol m⁻²s⁻¹.

FIG. 4. Light intensity dependent curves of water hydrolysis and O₂ evolution catalyzed by mCherry and GFP in the presence of TMBQ. TMBQ: 2,3,5-trimethyl-1,4-benzoquinone. Reaction system: 50 mM phosphate buffer (pH6.5), mCherry: 10 nM; GFP: 1 μM; TMBQ: 400 μM; light intensity: 0.6-1 μmol m⁻²s⁻¹.

FIG. 5. TMBQ concentration curves of O₂ evolution by fluorescent proteins GFP, YFP, CFP and BFP. 50 mM phosphate buffer (pH6.5), GFP: 1 μM; other fluorescent proteins: 10 nM; TMBQ: 400 μM; light intensity: 0.6-1 μmol m⁻²s⁻¹.

FIG. 6. Comparison of the activities of YFP (A) and mCherry (B) on water hydrolysis and O₂ evolution in the presence of different kind of quinones under light, BQ, 1,4-benzoquinone; MBQ, methyl-1,4-benzoquinone; DMBQ1, 2,5-dimethyl-1,4-benzoquinone; DMBQ2: 2,6-dimethyl-1,4-benzoquinone; TMBQ, 2,3,5-trimethyl-1,4-benzoquinone; DQ, duroquinone or tetramethyl-1,4-benzoquinone; UQ, 2,3-dimethoxyl-5-methyl-1,4-benzoquinone. Reaction system: 50 mM phosphate buffer, pH6.5; YFP, 10 nM; mCherry, 5 nM; light intensity: 0.6-1 μmol m⁻²s⁻¹.

FIG. 7. Absorption spectra of YFP (A, B) and mCherry (E, F) and their mutants. (B) is a zoom in view of YFPmu4 and YFPmu7 in (A). (F) is a zoom in view of mCherrymu4 and mCherrymu7 in (E). Fluorescence of YFP (C) and mCherry (G) and their mutant. Water hydrolysis and O₂ evolution activities of YFP (D) and mCherry (H) and their mutants under light. (A, B, C, E, F, G): absorption and fluorescence intensities were compared with same concentrations of proteins. Absorption intensity of non-mutated YFP or mCherry is considered as 1, and fluorescence intensity of non-mutated YFP or mCherry is considered as 100.

FIG. 8. Comparison of the sequences of different fluorescent proteins in GFP series.

FIG. 9. (A) Comparison of the O₂ evolution activities under light of YFP mutated in 232 amino acid residue and YFP mutant protein deleted from 232 amino acid residue. (B) Comparison of the O₂ evolution activities under light of GFP protein and GFP mutant protein deleted from 231 amino acid residue. The concentrations of the YFP L232H and the GFP were 1 μM. The concentrations of the other proteins were 10 nM. The concentration of TMBQ was 400 μM. The excitation light was 1-2 μmol m⁻²s⁻¹.

FIG. 10. Phylogenic tree of 110 fluorescent proteins from Cnidarian and Arthropoda (Alieva et al., 2008). Black spots indicate the fluorescent proteins used in this invention. These fluorescent proteins belong to different branch (A, B, C, D) of the phylogenic tree.

FIG. 11. (A) Phylogenic tree of all the LEAT proteins used in this invention, (B) Homologous comparison of all the LEAT proteins used in this invention. 1 is eqFP611, 2 is hcriCP, and so on.

FIG. 12. Map of plasmid pUC57 and its multiple cloning sites.

FIG. 13. (A) Map of vector pHB used for construction of plasmids for plant transformation. mCherry fragment was amplified by PCR, digested and ligated into pHB vector in the restriction sites of HindIII-XbaI. MCS: multiple cloning site. (B) Southern blot analysis of mCherry transgenic Brassica. (C) Observation of fluorescence in the root cells of transgenic plants. (D) RT-PCR and Western blot analysis of mCherry transgenic plant (L1-L6). (E) Analysis of the mCherry protein expression and subcellular localization by immuno-electromicroscopy. mCherry protein is mainly localized in the cytosol (Cy), nucleus (N). N, nucleus; CW, cell wall; Chl, chloroplast; V, vacuole; G, Golgi apparatus.

FIG. 14. (A) WT, empty vector control (pHB) and mCherry transgenic Brassica (L1-L3) were germinated and grown for one week in the phytotron under white light (PPFD:250 μmol m⁻²s⁻¹). Then the plants were moved and grown under white light, green light (PFD: 60 μmol m⁻²s⁻¹) or red+blue light (red light PFD; 60 μmol m⁻²s⁻¹, blue light PFD; 10 μmol m⁻²s⁻¹) for another 3 weeks and photos were taken, (B) Comparison of the fresh weights and dry weights of WT and mCherry transgenic Brassica grown under white light, green light or red+blue light for four weeks. Values in the figures are means±SD, for WT, n=10, for transgenic plant transformed with empty vector control pHB and mCherry, data were the averages of three independent lines and 10 plants for each line. **p≦0.01. (C) Net photosynthetic rate of WT and mCherry transgenic Brassica measured under different light qualities. Nine-week-old WT and mCherry transgenic Brassica grown in the phytotron (PPFD: 250 μmol m⁻²s⁻¹) were transferred and treated with different qualities of light for 4 days and net photosynthetic rate was measured. (D) Net photosynthetic rate of WT and mCherry transgenic Brassica grow in the natural field measured under different PPFD as light source. Net photosynthetic rate was measured in situ between 10:00 am and 12:00 pm. (E) Soluble protein contents in the mCherry transgenic Brassica were slightly increased.

FIG. 15. (A) Measurement of the fresh weight and dry weight per plant of 7-week-old WT and mCherry transgenic Brassica plants grown in the natural field conditions. Values in the figures are means±SD, n=10. *p≦0.05, **p≦0.01. (B) Comparison of the plant size, silique size, seed size, grain yield per plant and 1000-seed weight at the harvesting time. Values in the figures are means±SD, n=10. *p≦0.05, **p≦0.01.

FIG. 16. Transgenic Brassica showed enhanced light energy absorbance at different photon flux densities.

FIG. 17. (A) Measurement and comparison of the electron transfer rate, photochemical efficiency of PSII (Φ_{PSH}) and the excitation pressure of PSII (1−qL) of WT and mCherry transgenic Brassica grown under white light and red+blue light with different light intensities. 1−qL represents the redox state of the plastoquinone pool. (B) 77K Chl a fluorescence of thylakoid membrane. Thylakoid membrane extracted from WT leaves were excited with blue light (λ=435 nm) and green light (λ=540 nm) in the presence of 32 μg/ml of GST (Green_{GST}) or 32 μg/ml mCherry protein (Greenₘ). The inset shows the ratios of 695 nm (fluoresence of PSI) and 735 nm (fluoresence of PSII) excited with green light in the presence of mCherry to those in the present of GST, (C) 77K Chl a fluorescence of thylakoid membrane in the presence of different concentrations of mCherry protein. Black line (mCherry) represents the the intensity of fluorescence emission at 663 nm by GST-mCherry fusion protein. Values in (B) are the averages of six measurements±SD, n=6. Values in (C) are the averages of four measurements±SD, n=4. *p≦0.05, **p≦0.01. (D) Reduction kinetics of P700⁺ of WT and mCherry transgenic Brassica. The inset shows initial rate of P700⁺ reduction. This data suggested that the PSI cyclic electron transport capacity was increased in mCherry transgenic Brassica plants.

FIG. 18. mCherry protein expression enhances the intensity of the slow phase of millisecond-delayed light emission of Chl fluorescence. Measurement of the millisecond-delayed light emission of Chl fluorescence in WT and mCherry transgenic plants (TG) under white light and green light. The white light (PPFD: 1200 μmol m⁻²s⁻¹) was supplied by a halogen lamp, and the green light was obtained from the white light passing through a filter (530 nm<λ<560 nm).

FIG. 19. (A) Western blot analysis the expression of photosynthesis related protein β-subunit of ATP synthase (Atp B), D1 protein in reaction center of PSII, core protein of PSI Psa D, light harvesting complex II Lhcb1 subunit, light harvesting complex I Lhca1 subunit and cytochrome f (cytf). The results suggested that the contents of PSI, PSII and light harvesting complexes increased in the mCherry transgenic Brassica. (B) Initial and total activities of Rubisco in the leaves of WT and mCherry transgenic Brassica. (C) In situ measurement of state transition of leaves. For state transition measurement, the leaf was first illuminated with state 2 light with PFD of 100 μmol m⁻²s⁻¹ for 15 minutes then infrared light (6 μmol m⁻²s⁻¹) was turned on for 15 minutes, after which the infrared light was turned off for another 15 minutes. t_{0.5} represents the half time for transition between state 1 (St1) and state 2 (St2). (D) Measurement of the state transition of WT and mCherry transgenic Brassica grown under white light by 77K fluorescence emission. Thylakoid membrane was isolated from the WT and mCherry transgenic Brassica leaves grown under white light and the 77K fluorescence was measured. (E) Measurement of the state transition of WT and mCherry transgenic Brassica grown under red+blue light by 77K fluorescence emission. All spectra were normalized at 685 nm. Values in (B-D) are means±SD, n=6. *p≦0.05, **p≦0.01. Data suggested that the ability of light harvesting complex in regulating the two photo systems is significantly increased.

FIG. 20. Chlorophyll contents and the Chl a/b ratio (A) and carotenoid contents (B) in mCherry transgenic Brassica leaves. V: violaxanthin, L: lutein, Z: zeaxanthin, A: Antheraxanthin, N: neoxanthin. Leaves were from WT and mCherry transgenic Brassica plants grown either under white light in phytotron (PPFD: 250 μmol m⁻²s⁻¹, 9-week-old plants) or in the natural field (11-week-old plants). Data in the figure represent mean±SD, n=6, *p≦0.05.

FIG. 21. (A) Measurement of the intercellular CO₂ concentration in WT and mCherry transgenic Brassica grown under white light (PPFD: 250 μmol m⁻²s⁻¹) in phytotron. Data in the figure are means of 6 different lines±SD. (B) Measurement of the stomatal conductance (Gs) of WT and mCherry transgenic Brassica grown under white light (PPFD: 250 μmol m⁻² s⁻¹) in phytotron. Data in the figure are means of 6-8 leaves±SD, *p≦0.05. (C) Intercellular CO₂ concentration and stomatal conductance of leaves from 11-week-old Brassica grown in the field. Data in the figure are means of leaves from 6 different lines and 6 leaves from each line±SD. *p≦0.05. (D) The intercellular CO₂ concentration response curve of net photosynthetic rate under saturated light. The dashed lines represent the state that the photosynthesis is limited by the Rubisco carboxylation rate in plant, and the solid fit lines represent the state of RuBP regeneration limitation. Data in the figure are the average of four measurements±SD.

FIG. 22. Changes of photochemical efficiency of PSII (Φ_{PSII}) in WT and mCherry transgenic Brassica after being transferred from red+blue light to white light. (B) NAD⁺ and NADH contents in leaves grown under red+blue light, white light and 2 h after being transferred from red+blue light to white light, (C) NADP⁺ and NADPH contents in leaves grown under red+blue light, white light and 2 h after being transferred from red+blue light to white light. (D) GSH and GSSG contents in leaves grown under red+blue light, white light and 2 h after being transferred from red+blue light to white light. (E) Ascorbic acid (ASC) and dehydroascorbic acid (DHA) contents in leaves grown under red+blue light, white light and 2 h after being transferred from red+blue light to white light. Data in the figures are the averages form 5 measurements±SD, n=5. *p≦0.05, **p≦0.01.

FIG. 23. Comparison of the respiration rate of the etiolated hypocotyl of mCherry transgenic Brassica under light or in dark and the net O₂ evolution rate when respiration was blocked. −HgCl₂: difference in respiration rate before and after light being switched on when HgCl₂ was absent. +HgCl₂: the net O₂ evolution rate when respiration was blocked when HgCl₂ was added. Data in the figure are average of measurements±SD, n=4.

FIG. 24. mCherry transgenic rice grows obviously faster at seedling stage. (A) Identification of mCherry transgenic rice by PCR analysis. The numbers underlined indicate the positive transgenic plants identified by PCR. “−” is the negative controls for PCR and “+” is positive control for PCR, which was amplified with pHB-mCherry plasmid as template. Mr, molecular marker. (B) Growth of mCherry transgenic rice at seedling stage. mCherry transgenic rice grows faster than WT.

FIG. 25. (A) Identification of mCherry transgenic wheat by PCR analysis. The numbers underlined indicate the positive transgenic lines. Mr indicates the molecular marker. (B) Comparison of the mCherry transgenic wheat with corresponding WT cultivars (Jia, Xiaoyan 54 and Jing 411). The size of seed and spike of mCherry transgenic wheat are significantly increased as compared to WT. (C) Comparison of the grain yield per plant of mCherry transgenic wheat with corresponding WT cultivars (Jia, Xiaoyan 54 and Jing 411). Data in the figure are means±SD, n=3, *p≦0.05.

FIG. 26. (A) Identification of BFP transgenic Brassica, Genomic DNA was extracted from Brassica leaves, the BFP fragment was integrated into the genome and the integration was identified by PCR. pHB-BFP is PCR positive control and amplification was carried out with pHB-BFP plasmid as template. B1-B5 are the positive BFP transgenic lines. (B) RT-PCR analysis of BFP expression in BFP transgenic Brassica leaves. Total RNA was extracted from WT and B1-B5 lines of BFP transgenic Brassica. RNA was reverse transcribed into cDNA and BFP expression was analyzed by RT-PCR. UBI was used as internal control of RT-PCR to evaluate the expressional level of BFP in the tested plants. (C) Growth of 7-week-old WT and BFP transgenic Brassica in the field. The fresh weight per plant and dry weight per plant of WT and BFP transgenic Brassica were measured. Data in the figure are means±SD, n=10. *p≦0.05, **p≦0.01. (D) Comparison of the plant morphology, the size of siliques and seeds, grain yield per plant at harvest time. Data in the figure are means±SD, n=10. *p≦0.05 and **p≦0.01.

FIG. 27. WT, empty vector control (pHB) and BFP transgenic Brassica (B1-B5) were germinated and grown in the phytotron under white light (250 μmol m⁻²s⁻¹, UV-B intensity: 0.013 mW cm⁻²) for one week. Then the seedlings were moved to grow under white for another three weeks (A) or under white light+UV conditions (UVB intensity 0.075 mW cm⁻²) for three weeks. Photographs were taken three weeks after moved. The result suggested that, the resistance to UVB was increased in BFP transgenic Brassica.

FIG. 28. Measurement of the net photosynthetic rate of WT and BFP transgenic Brassica under different PPFD. Net photosynthetic rate was measured in situ between 10:00 am and 12:00 pm. Light source for net photosynthetic rate measurement is sunlight. The result suggested that transgenic BFP Brassica enhanced leaf photosynthesis preferentially under high light conditions.

FIG. 29. (A) Comparison of the photochemical efficiency of PSII (Φ_{PSII}) between WT and BFP transgenic Brassica. (B) Comparison of the excitation pressure of PSII (1−qL) between WT and BFP transgenic Brassica. 1−qL represents the redox status of the plastoquinone pool. (C) The reduction kinetics of P700⁺ of WT and BFP transgenic Brassica. (Inset) The initial rate of P700⁺ reduction, which represents the PSI cyclic electron transport capacity.

FIG. 30. Western blot analysis of the photosynthesis related protein β-subunit of ATP synthase (Atp B), D1 protein in reaction center of PSII, core protein of PSI Psa D, light harvesting complex II Lhcb1 subunit, light, harvesting complex I Lhca1 subunit and cytochrome f (cytf) in WT and BFP transgenic Brassica. The results suggested that the contents of PSI, PSII and light harvesting complexes increased in the BFP transgenic Brassica.

FIG. 31. WT and BFP transgenic Brassica were grown in the phytotron for 11 weeks with sunlight and halogen lamp as light source (400 μmol m⁻²s⁻¹, with UV). Chlorophyll and carotenoid contents were measured with leaves from the seedlings. (A) Chl a/b ratio in leaves. The Chl a/b ratio is significantly higher in BFP transgenic Brassica than that in WT. (B) Carotenoid contents in leaves. V: violaxanthin, L: lutein, Z: zeaxanthin, A: Antheraxanthin, N: neoxanthin. Carotenoids are accessory photosynthetic pigments. Increase in caroteinoid contents may increase the dissipation of light energy under high light conditions and increase the light protection ability of plants. (C) The stomatal conductance of BFP transgenic Brassica and WT. (D) Non-photochemical quenching (NPQ) of BFP transgenic Brassica and WT. The result suggested that the light protection ability of BFP transgenic Brassica was significantly higher than that of WT. Data in the figure are means±SD, n=6, *p≦0.05.

FIG. 32. (A) Identification of BFP transgenic wheat by PCR analysis. The numbers underlined indicate the positive transgenic lines. (B) Comparison of the BFP transgenic wheat with corresponding WT cultivars (Jia, Xiaoyan 54 and Jing 411). The size of seed and spike of BFP transgenic wheat are significantly increased as compared to WT. (C) Comparison of the grain yield per plant of BFP transgenic wheat with corresponding WT cultivars (Jia, Xiaoyan 54 and Jing 411). Datas in the figures are means±SD, n=3, *p≦0.05, **, P<0.01.

FIG. 33. (A) Identification of mGFP5 transgenic wheat by PCR analysis. The numbers underlined indicate the positive transgenic lines. Mr, molecular marker. (B) The size of seed and panicle is significantly increased, and the panicle number is increased of mGFP5 transgenic wheat as compared to those of WT cultivar Jia.

FIG. 34. (A) Identification of mGFP5 transgenic rice by PCR analysis. The numbers underlined indicate the positive transgenic lines. Mr, molecular marker. (B) Comparison of mGFP5 transgenic rice and WT. Left, grain yield per plant, Right, 100-seed-weight. Zhonghua 11 cultivar is the WT, mGFP5-1 and mGFP5-2 are two different mGFP5 transgenic lines. Statistic analysis showed that ectopic expression of mGFP5 in rice significantly increased the grain yield per plant and 100-seed-weight. Data represent means of three replicates. Standard error is from three plots and 3 plants from each plot. (*, p≦0.05, **, p≦0.01).

FIG. 35. (A) Identification of transgenic cotton by PCR analysis. The numbers underlined indicate the positive transgenic plants. Mr, molecular marker. (B) Comparison of the growth of cotton, the biomass per plant, the number of bolls per plant and the weight of bolls per plant. The biomass, the size, number and weight of bolls are significantly increased in mGFP5 transgenic cotton.

FIG. 36. (A) Identification of transgenic Brassica expressing low fluorescent mutant proteins mCherrymu3 (mu3), mCherrymu4 (mu4), mCherrymu5 (mu5) and YFPmu7 (mu7) by PCR analysis, Mr, molecular marker, “+” is the positive control for PCR, which was amplified using pHB-mCherrymu4 plasmid as template. (B) Comparison of transgenic Brassica expressing low fluorescent mutant proteins mCherrymu3, mCherrymu4, mCherrymu5 and YFPmu7 with WT and vector control pHB transgenic Brassica. At the seedling stage, transgenic Brassica expressing low fluorescent mutant proteins grow more vigorously than WT.

FIG. 37. Model of water photohydrolysis catalyzed by LEAT proteins. LEAT protein absorbs light, and converts itself into an excitation state (LEAT*), then catalyzes the water hydrolysis and returned to ground state. During water hydrolysis, the generated electron and proton are transferred to quinone, expecially plastoquinone and reslult in quinone reduction and and O2 evolution. LEAT: LEAT protein at the ground state; Q: quinone. QH₂: hydroquinone; LEAT*: LEAT protein at the excitation state.

### DESCRIPTION OF THE PREFERRED EMBODIMENTS

After in-depth studies, the inventor develops a new method for increasing light energy utilization efficiency of a plant, the method is carried out by expressing exogenous Light Energy Absorption and Transduction protein (LEAT protein) in the plant, catalyzing water hydrolysis under light and transferring the electrons and protons generated from the hydrolysis to quinones, such as plastoquinones that exist in a plant, therefore accomplishing the O₂ evolution. This process provides consecutive electron sources under light, regulates the redox status in the plant, changes the expression of the photosynthesis-related genes in the plant and increases photosynthesis efficiency of the plant.

### Terms

As used herein, “plant” refers to (a) plant(s) containing photosynthetic organs and is suitable for gene transformation operations. Said “plant” includes whole plant, descendant and portion of the plant (including seed, branch, stem, leaf, root (including tuber), flower, and tissues and organs), plant cells, suspension cultures, callus, embryo, meristem, gametocyte, sporophyte, pollen and sporule. The plant can be, for example, but not limited to gymnosperms, monocotyledonous or dicotyledonous plants. More particularly, the plant includes, but not limited to wheat, barley, rye, rice, corn, sorghum, sugar beet, apple, pear, plum, peach, apricot, cherry, strawberry, raspberry, blackberry, bean, lentil, pea, soybean, Brassica, mustard, poppy, Artemisia carvifolia, olea, sunflower, coconut, castor-oil plant, cocoa bean, peanut, calabash, tobacco, oil palm, cucumber, watermelon, cotton, flax, hemp, jute, orange, lemon, grapefruit, spinach, lettuce, asparagus, cabbage, Chinese cabbage, pakchoi, carrot, onion, potato, tomato, green pepper, avocado, cassia, camphor, tobacco leaf, nut, coffee, eggplant, sugar cane, tea, pepper, vine, oysters flax grass, banana, poplar, willow, pine, fir, eucalyptus, Euphorbia lathylris, Euphorbia tirucalli, Metroxylon sagu, Jojoba, natural rubber tree and ornamental plants.

As used herein, the “improved trait” refers to the characters of an improved plant, including, but not limited to increasing light absorption efficiency of a plant, increasing CO₂ utilization, increasing light transfer efficiency, increasing photosynthetic assimilation efficiency, enhancing light protection mechanism, number and size of organs, plant structure (number of tillers or panicles), seed number, size of seeds or panicles, increasing plant economic yield, seed size, weed weight, plant branch number, plant bearing number, biomass of a plant and/or plant yield, etc. In an important aspect of the invention, the improved trait is increasing plant yield, including the high yield without environmental stress conditions or under environmental stress conditions. The environmental stress conditions may comprise, for example, lack of sunshine, high light intensity, high ultraviolet radiation, high temperature, and high plant density. “Yield” can be affected by many plant characters including light absorption efficiency, light transfer efficiency, photosynthetic carbon assimilation efficiency, biomass accumulation, number and size of organs, plant structure (number of tillers or panicles), seed number, size of seeds or panicles of a plant and the like. Moreover, the transformed plant obtained only for the purpose of labeling or tracing certain structures or certain proteins in plant tissues or cells is not included in the scope of “plant with an improved trait”.

As used herein, “improvement of (a) plant(s) trait(s)”, “improved trait(s)”. “improved plant trait(s)”, “trait(s) improvement”, “plant trait(s) improvement” can be used interchangeably, which refer to increasing light absorption efficiency, increasing light transfer efficiency, increasing photosynthetic carbon assimilation efficiency, enhancing light protection mechanism, favorable change of number or size of organs, favorable change of plant structures (number of fillers or panicles), increasing seed number, favorable change of seeds or panicles, increasing crop economic yield, increasing seed size, increasing seed weight, favorable change of plant branch number, increasing plant bearing number, increasing biomass of the plant and/or increasing plant yield and the like in the improved plant of the invention compared with a plant prior to the improvement.

As used herein, the term “yield” may involve the vegetative biomass of a plant (biomass of root and/or branch, leaf) or the biomass of propagative organ and/or propagule (such as seed) of a plant.

“Yield-related trait” includes but not limited to seed number, seed size, seed weight, plant branch number, plant bearing number, plant biomass, and plant yield, etc.

As used herein, the terms “increasing”, “improving” or “enhancing” can be used interchangeably and refer to increasing at least 2%, 3%, 4%, 5%, 6%, 7%, 8%, 9% or 10%, preferably at least 15% or 20%, more preferably 25%, 30%, 35% or 40% or more in the yield and/or growth or other agricultural traits compared with the control plant defined herein in the application.

As used herein, the terms “seed index” or “100-seed weight” can be used interchangeably and both refer to the weight per 100 seeds, the data of which reflects the size and plumpness of a seed.

As used herein, various methyl quinone derivatives exist widely in organisms, some of which are intermediate product of metabolic process; some of which participate in the regulation of various basal metabolisms and secondary metabolisms in organisms, wherein the “plastoquinone (PQ)” exist in the plant is a methyl quinone derivative. There are two methyl groups linked to the quinone ring and a branch chain linked with varying numbers of isoprene unit. There are several types of PQs exist in the plant, which differ from each other by the number of isoprene unit. PQ exists widely in chloroplast and cytosol such as rough endoplasmic reticulum. As another example, phylloquinone is a methyl quinine derivative which exists widely in many membrane structures in plant chloroplast and cytosol.

“Light” as used herein, in addition to the electromagnetic wave within the visible light at a wavelength within the range of 400 to 760 nm, also refers to ultraviolet ray at a wavelength within the range of 300 to 400 nm and near infrared ray at a wavelength within the range of 760 to 1000 nm. The “light energy” refers to the energy of electromagnetic wave within the above range as 300 to 1000 nm.

As used herein, the “Light Absorption and Transduction Protein (LEAT protein)” is a protein which absorbs the electromagnetic wave at the wavelength of 300 to 1000 nm by the chromophore constituted by the amino acid residues which form the protein sequence, and transfer the absorbed energy of the electromagnetic wave (as the use above, briefly “light energy” hereafter) into chemical energy. The process of transfer into chemical energy is as follows: upon light energy absorption, the electron and proton generated by catalyzing water hydrolysis are transferred to related methyl quinone and the derivatives thereof, and reslult in methyl quinone and the derivatives thereof reduction. The methyl quinone and the derivatives thereof include TMBQ (2,3,5-trimethyl-1,4-benzoquinone), DMBQ2 (2,6-dimethyl-1,4-benzoquinone), MBQ (methyl-1,4-benzoquinone) and DMBQ1 (2,5-dimethyl-1,4-benzoquinone), preferably TMBQ (2,3,5-trimethyl-1,4-benzoquinone).

The lower limit of the wavelength of the electromagnetic wave absorbed by LEAT is 300 nm, preferably 320 nm, 340 nm, 350 nm, 360 nm, more preferably, 370 nm, 380 nm, 390 nm, 400 nm, 410 nm or 420 nm; the upper limit is 1000 nm, preferably 950 nm, 900 nm, 850 nm, 800 nm, more preferably, 750 nm, 700 nm, 680 nm, 660 nm, 650 nm, 640 nm, 630 nm or 620 nm.

The LEAT protein is preferably selected from:

Fluorescent protein or its mutant proteins with one or more (such as 1 to 30, preferably 1 to 20, more preferably 1 to 10, even more preferably 1 to 5) amino acid site mutations which are changed in fluorescence intensity and fluorescence emission spectra but can still utilize light energy to catalyze water hydrolysis and reduction of methyl quinine derivatives such as 2,3,5-trimethyl-1,4-benzoquinone or the analogues thereof; or

Non-fluorescent chromoprotein or its mutant proteins with one or more (such as 1 to 30, preferably 1 to 20, more preferably 1 to 10, even more preferably 1 to 5) amino acid site mutations which can still utilize light energy to catalyze water hydrolysis and reduction of methyl quinine derivatives such as 2,3,5-trimethyl-1,4-benzoquinone or the analogs thereof.

The fluorescent protein refers to a natural or synthetic protein, which can emit fluorescence upon excited by the light at certain wavelengths without the need of additional cofactors.

The non-fluorescent chromoprotein refers to a natural or synthetic protein, which can absorb the light at certain wavelengths without the need of additional cofactors, and do not emit fluorescence thereafter. A chromoprotein which contains any prosthetic group or cofactor, or absorbs light energy by any prosthetic group or cofactor, such as hemoglobin, flavoprotein, cytochrome, etc, does not fall into the scope of the non-fluorescent chromoprotein of this invention.

The fluorescent protein and non-fluorescent chromoprotein above both have similar three-dimensional cylindrical structure, wherein most of the polypeptide backbone is folded into 11 β-sheets linked with hydrogen bonds, alpha helix containing chromophore is in the center, both can form chromophore by their amino acid sequence to absorb and emit light energy.

The fluorescent protein of the invention is preferably selected from: Blue Fluorescent Protein, Cyan Fluorescent Protein, Green Fluorescent Protein, Yellow Fluorescent Protein, Red Fluorescent Protein, Far-red Fluorescent Protein, and Near Infra-red Fluorescent Protein.

The “Blue Fluorescent Protein (BFP)” is a fluorescent protein with an emission peak at 440-470 nm; for example, the Blue Fluorescent Protein has an amino acid sequence as set forth in SEQ ID NO: 4,

The “Cyan Fluorescent Protein (CFP)” is a fluorescent protein with an emission peak at 470-500 nm; for example, the Cyan Fluorescent Protein has an amino acid sequence as set forth in SEQ ID NO: 10 or SEQ ID NO: 36.

The “Green Fluorescent Protein (GFP)” is a fluorescent protein with an emission peak at 500-525 nm; for example, the Green Fluorescent Protein has an amino acid sequence as set forth in SEQ ID NO: 6, SEQ ID NO: 28, SEQ ID NO: 40 or SEQ ID NO: 44.

The “Yellow Fluorescent Protein (YFP)” is a fluorescent protein with an emission peak at 525-570 nm; for example, the Yellow Fluorescent Protein has an amino acid sequence as set forth in SEQ ID NO: 8 or SEQ ID NO: 50.

The “Red Fluorescent Protein (RFP)” is a fluorescent protein with an emission peak at 570-630 nm; for example, the Red Fluorescent Protein has an amino acid sequence as set forth in SEQ ID NO: 2, SEQ ID NO: 30, SEQ ID NO: 34, SEQ ID NO: 42 or SEQ ID NO: 46.

The “Far-red Fluorescent Protein” is a fluorescent protein with an emission peak at 630-760 nm.

The “Near Infra-red Fluorescent Protein” is a fluorescent protein with an emission peak at 760-900 nm; for example, the Near Infra-red Fluorescent Protein has an amino acid sequence as set forth in SEQ ID NO: 32 or SEQ ID NO: 48.

The “non-fluorescent chromoprotein” is a non-fluorescent chromoprotein has an amino acid sequence as set forth in SEQ ID NO: 38.

As used herein, “expression” refers to the transcription from (one or more) polynucleotide or an expression cassette containing a polynucleotide to mRNA, with or without the subsequent translation of said mRNA. This process comprises the transcription of DNA and the processing of the resulted mRNA.

As used herein, the terms “introduction” or “transformation” comprise transforming exogenous polynucleotide(s) into a host cell, without consideration of the methods used in transformation. All the plant tissues which can be clonal proliferated by organogenesis or embryo genesis can be transformed by the cassette of the invention, thereby regenerating the whole plant. The selection of the specific tissue may vary by the available and most suitable clonal proliferation system for the specific species to be transformed. Exemplary tissue targets include leaf disc, pollen, embryo, cotyledon, hypocotyl, female gametophyte, callus, existing meristem (such as apical meristem, axillary and roof meristem), and induced meristem (e.g., cotyledon meristem and hypocotyl meristem). A Polynucleotide can be introduced into a host cell transiently or stably, and can maintain in non-integrative state, for example, as a plasmid. Optionally, the polynucleotide(s) can integrate into the host genome. The resulted transformed plant cell can be regenerated into a transformed plant by the methods known for those skilled in the art.

The transformation of exogenous genes into plant genome is known as transformation. Transform the gene(s) into the plant species is a quite common technology. Advantageously, any of the several transformation methods can be used to transform a target gene into a proper progenitor cell. Transient or stable transformation can be performed by disclosed transformation methods and methods for regenerating a plant from a plant tissue or cell. Transformation methods comprise: liposome mediated transformation, electroporation, chemicals that increasing free DNA uptake, direct injection of DNA into a plant, gene gun/particle bombardment, transformation with virus or pollen, and microparticle bombardment. The methods can be selected from the calcium/polyethylene glycol method for protoplast (Krens, F. A. et al., (1882) Nature 296, 72-74; Negrutiu I. et al., (1987) Plant Mol. Biol. 8:363-378); electroporation for protoplast (Shillito R. D. et al., (1985) Rio/Technol 3, 1099-1102); microinjection for plant materials (Crossway A. et al., (1986) Mol. Gen Genet 202: 179-185); DNA or RNA coated particle bombardmen (Klein T. M. et al., (1987) Nature 327:70); infection with (non-conformity) virus, etc. Transformation mediated by Agrobacterium tumefaciens is preferred to generate transgenic plants (including transgenic crop plants). In planta transformation is an adventageous transformation method. To this end, for example, Agrobacterium tumefaciens can be applied in a seed of a plant, or Agrobacterium tumefaciens can be inoculated into plant meristem. It has been proved that applying the transformed Agrobacterium tumefaciens suspension to the whole plant or at least flower primordium is particularly advantageous. Then the plant is cultured until the seed of said plant is obtained (Clough and Rent, Plant J. (1998) 16, 735-743).

As to “control plant”, selection of a suitable control plant is a routine portion of experimental design, which can include corresponding wild type plant or corresponding plant without target gene. In general, a control plant is the same plant species or even the same variety as the plant to be assessed. A control plant can also be the one losses transgenic plant by separation. As used herein, the control plant does not only refer to the whole plant, but the part of the plant as well, including seed and part of seed.

The terms “increasing expression”, “over expression” and “ectopic expression” used herein refer to any form of extra expression relative to the original wild type expression level.

As used herein, “expression cassette” herein refers to a recombinant DNA molecule, which contains the expected nucleic acid coding sequence encoding Light Energy Absorption and Transduction protein; the DNA molecule further contains necessary or expected suitable regulatory elements for operable link to the coding sequence in vitro or in vivo. “Regulatory element” herein refers to a nucleotide sequence which can control the expression of a nucleic acid. Exemplary regulatory elements comprise a promoter, a transcription terminator or an upstream regulatory region, and these regulatory elements facilitate duplication, transcription, post-transcriptional modification of a nucleic acid. Moreover, regulatory elements may also comprise: enhancer, Internal Ribosomal Entry Site (IRES), origin of replication, and polyadenylation signal, etc.

As used herein, the “operable link” or “operably linked to” refer to a state that certain parts of a linar DNA sequence can regulate or control the activity of other parts of the same linar DNA sequence. For example, a promoter is operable linked to a coding sequence if it controls the transcription of the sequence.

As used herein, “exogenous” or “heterologous” genes or proteins refer to the genes or proteins non-naturally included in the organism genome. The “exogenous protein coding gene” is also referred as “heterologous DNA”, which refers to one DNA molecule or a group of DNA molecules which does not originally exist in a certain host cell; or refers to a DNA molecule different from the DNA molecule in a certain host cell.

As used herein, the “contain”, “possess” or “comprise” emcompass “include”, “consist mainly of”, “consisting essentially of” and “consist of”; the “consist mainly of”, “consisting essentially of” and “consist of” are the specific terms of “contain”, “possess” or “comprise”.

### LEAT Protein

The inventor unexpectedly finds that a series of Light Energy Absorption and Transduction proteins (LEAT proteins) can catalyze the hydrolysis of the water and the reduction of the plastoquinone or analogue thereof (such as 2,3,5-trimethyl-benzoquinone), the methyl quinine derivatives (e.g., 2,3,5-trimethyl-benzoquinone or plastoquinone) under light, and release O₂. Therefore, the LEAT protein is over expressed in a plant to catalyzes the water hydrolysis, and the generated electron thereby is used to the reduction of various types of methyl quinone derivatives, and the reduced methyl quinone derivatives participate in various oxidation-reduction reactions in vivo, regulates the expression of relative genes, promotes plant growth and improves plant traits.

A variety of Light Energy Absorption and Transduction proteins (LEAT proteins) can be used in the invention, as long as the protein can absorb energy from the photon and get enough energy for water hydrolysis, or the protein can absorb light at the wavelength of 300 to 1000 nm. It is common knowledge in the art that the hydrolysis of one H₂O molecule into 2 electrons and 2 protons requires 1.23 eV of light energy; therefore, as long as the energy absorbed from photon by the LEAT protein is more than the energy for water hydrolysis, as 1.23 eV of light energy, the reaction can be driven and the generated electrons and protons are mediated by the quinine compounds to reduce the oxidizing materials in vivo. Therefore, the invention relates to a series of proteins with light energy absorption and transduction function, including the protein gene of absorbing light radiation with and without fluorescence emission, such as Green Fluorescent Protein, Yellow Fluorescent Protein, Red Fluorescent Protein and the mutants thereof, as they can be used to increase light energy utilization efficiency of a plant and significantly increase biomass.

As the preferred embodiment of the invention, the LEAT proteins comprise Blue Fluorescent Protein, Cyan Fluorescent Protein, Green Fluorescent Protein, Yellow Fluorescent Protein, Red Fluorescent Protein or Far-red Fluorescent Protein or non-fluorescent chromoprotein.

Fluorescent proteins are a kind of proteins able to emit light under suitable conditions and the amino acid residues of said protein form into its chromophore. In current technologies, Fluorescent proteins are mainly used to label cell structure and monitor intracellular process, and they are also used in tracing the cell populations such as tumor cells in vivo. The earliest Green Fluorescent Protein (GFP) was found in a jellyfish (Aequorea Victoria) in 1962 and thereafter GFP is separated from marine coral polyp. A series of fluorescent proteins with different spectral characteristics were found in Actinozoa of Coelenterata such as coral and sea anemone in the subsequent researches. All the fluorescent proteins have a similar three-dimensional cylindrical structure, wherein most of the polypeptide backbone is folded into 11 β-sheets linked with hydrogen bonds, and an alpha helix containing chromophore is in the center. So far, a series of derivatives have been developed by the transformation of fluorescent proteins with genetic engineering means, their emission spectrum has almost covered the whole visible region (400 to 760 nm) and near infra-red region (760 to 900 nm). Therefore, the fluorescent proteins preferably have a three-dimensional structure, wherein an α-helix containing chromophore is surrounded by a beta barrel consisting of 11 β-sheets.

The fluorescent protein refers to natural or synthetic proteins, wherein the chromophore consisting of their amino acids can be excited by electromagnetic wave at certain wavelength range and emits visible light without an additional cofactor. In another aspect, the fluorescent protein can be a fluorescent protein or the derivatives thereof separated from Coelenterata, such as jellyfish, coral polyp or sea anemone; In another aspect, the fluorescent protein is Green Fluorescent Protein (Swiss-Prot: P42212) and the derivatives thereof from Aequorea, such as BFP as set forth in SEQ ID NO: 4; the fluorescent protein can also be red fluorescent protein (DsRed) (Swiss-Prot:Q9U6Y8) from Discosoma sp. or the derivatives thereof, such as mCherry.

The non-fluorescent chromoprotein of the invention has similar structure with above fluorescent protein, and can absorb light energy at certain wavelength, but the wild type protein has very poor ability to emit fluorescence.

At present, GFP, RFP, BFP and the like are widely used in biological imaging research, and report the location of the protein in tissues and cells (Shaner N C, Patterson G H, Davidson M W. 2007 Advances in fluorescent protein technology. J Cell Sci. 15;120 (Pt 24): 4247-4260; mCherry Shaner N C, Campbell R E, Steinbach P A, Giepmans B N, Palmer A E, Tsien R Y. (2004) Improved monomeric red, orange and yellow fluorescent proteins derived from Discosoma sp, red fluorescent protein, Nat Biotechnol. 22(12):1567-72). However, at present these fluorescent proteins or non-fluorescent chromoproteins have not been used in the preparation of transgenic plants to increase photosynthesis efficiency of a plant.

As a preferred embodiment of the invention, the LEAT protein is Blue Fluorescent Protein (BFP), Cyan Fluorescent Protein (CFP), Green Fluorescent Protein (GFP), Yellow Fluorescent Protein, Red Fluorescent Protein (RFP), or Far-red Fluorescent Protein or non-fluorescent chromoprotein. The variants of above fluorescent proteins can also be used in the invention. Although fluorescence intensity changes largely, the variants of above fluorescent proteins can still catalyze the hydrolysis of water and the release of O₂ under light in the presence of methyl quinones or the derivatives thereof, the reducing energy is generated sustainably and stored in the quinones, thus they can also be used in the invention.

As another preferred embodiment of the invention, the fluorescent protein is selected from, but not limited to Yellow Fluorescent Protein, Red Fluorescent Protein (RFP), or Far-red Fluorescent Protein. It is to be understood that, the method of the invention achieves the technical effect by using the fluorescent protein to transfer wavelength or spectrum energy of absorbed light, thus, any fluorescent protein that can be excited by light at the wavelength of 495 to 620 nm and has an emission peak at 550 to 700 nm has similar optical characteristics to RFP, that transfers the light of low plant utilization efficiency into the light of high photosystem utilization efficiency, thereby the protein can be used in the invention, for example, mHoneydew (excitation peak at 487/504 nm, emission peak at 537/562 nm), mBanana (excitation peak at 540 nm, emission peak at 553 nm), mOrange (excitation peak at 548 nm, emission peak at 562 nm), dTomato (excitation peak at 554 nm, emission peak at 581 nm), tdTomato (excitation peak at 554 nm, emission peak at 581 nm), mStrawberry (excitation peak at 574 nm, emission peak at 596 nm), mCherry (excitation peak at 587 nm, emission peak at 610 nm), mPlum (excitation peak at 590 nm, emission peak at 649 nm), mRFP1 (excitation peak at 584 nm, emission peak at 607 nm), mTangerine (excitation peak at 568 nm, emission peak at 585 nm).

The “Green Fluorescent Protein”, “Cyan Fluorescent Protein”, “Blue Fluorescent Protein” also include “enhanced Green Fluorescent Protein”, “enhanced Cyan Fluorescent Protein”, “enhanced Blue Fluorescent Protein”.

The “Cyan Fluorescent Protein” can have or be essentially identical to, for example, the amino acid sequence as set forth in GenBank Accession No: AAQ96626; or the protein formed by one or more amino acid residue substitution, deletion or addition in this amino acid sequence, and having identical functions to said protein with said amino acid sequence; or the protein have more than 70% identity with the amino acid sequence as set forth in GenBank Accession No: AAQ96626, and having improved trait functions.

The “Yellow Fluorescent Protein” can have or be essentially identical to, for example, the amino acid sequence as set forth in GenBank Accession No: ADR00308; or the protein formed by one or more amino acid residue substitution, deletion or addition in this amino acid sequence, and having identical functions to said protein with said amino acid sequence; or the protein have more than 70% identity with the amino acid sequence as set forth in GenBank Accession No: ADR00308, and having improved trait functions.

The “Far-red Fluorescent Protein” can have or be essentially identical to, for example, the amino acid sequence as set forth in GenBank Accession No: ACH06541; or the protein formed by one or more amino acid residue substitution, deletion or addition in this amino acid sequence, and having identical functions to said protein with said amino acid sequence; or the protein have more than 70% identity with the amino acid sequence as set forth in GenBank Accession No: ACH06541, and having improved trait functions.

The “non-fluorescent chromoprotein” can have the amino acid sequence as set forth in GenBank Accession No: DQ206394 (gfasCP), AF363776 (hcriCP), AY485336 (anm2CP), etc.

The LEAT proteins used in the invention can be naturally occurring, for example, it can be isolated or purified from lower organisms, such as Coelenterata. Moreover, the LEAT protein can also be artificially prepared, for example, it can be produced by common genetic engineering technologies. Preferably, a recombinant LEAT protein can be used in the invention. Any suitable LEAT protein can be used in the invention. The LEAT protein comprises the full length of the LEAT protein or the biological active fragments thereof. The protein is formed by one or more (such as 1 to 30, preferably 1 to 20, more preferably 1 to 10, even more preferably 1 to 5) amino acid residue substitution, deletion or addition in the amino acid sequence of wild type LEAT protein, and has identical functions to the protein with this sequence; or the protein has more than 70% sequence identity with the protein with wild type amino acid sequence, and has identical functions to the wild type protein. The LEAT protein or the biological active fragments thereof comprise a part of conservative amino acid replacement sequence, and said sequence with amino acid replacements does not affect its features on light energy absorption and transduction. Appropriate amino acid replacement is a well known technology in the art, and the technology is readily performed and ensures not changing the biological activity of the resulted molecule. These technologies make those skilled in the art recognize that changing a single animo acid in non-essential region of a polypeptide generally will not alter the biological activity; see Watson et al., Molecular Biology of The Gene, 4^{th} edition, 1987, The Benjamin/Cummings Pub. Co. P224. Any biological active fragment of LEAT protein can be used in the invention. The biological active fragment of LEAT protein herein refers to a polypeptide which still maintains all or part of the function of the full length LEAT protein. In general, the biological active fragment maintains at least 50% of the activity of full length LEAT protein. Under more preferred conditions, the active fragment maintains 60%, 70%, 80%, 90%, 95%, 99% or 100% of the activity of full length LEAT protein. Improved or modified LEAT proteins can also be used in the invention, such as the LEAT protein improved or modified to enhance its half-life, effectiveness, metabolism and/or effect of protein can also be used. It is said that any form that does not affect the light energy absorption and transduction of the LEAT protein can be used in the invention.

The LEAT protein of the invention can further be used to improve plant traits in several aspects, comprising: increasing light energy utilization efficiency of a plant; increasing the photochemical efficiency of PSII or PSI of a plant; increasing plant photosynthetic electron transfer efficiency; increasing the efficiency of CO₂ assimilation; increasing plant net photosynthesis rate; enhancing the light protection of the plant photosynthetic apparatus; increasing plant growth; increasing the biomass of a plant; increasing the number of seeds or panicles; increasing the total protein content; increasing the size of seeds or panicles and/or increasing plant economic yield. The above plant traits variety is very beneficial for the improvement of plant species.

### Methods for Improving Plant Traits

The utilization of sunlight by a plant is not full spectrum utilization; the plant absorbs light energy at certain wavelength dependent on different chlorophyll molecules. For example, the Chl a/b contained in higher plants mainly absorbs red and blue-violet light with lower utilize light energy at other part of spectrum such as yellow light and lowest utilize of green light. The invention utilizes the characteristic that different LEAT proteins can absorb photons at certain wavelength and catalyze water hydrolysis which generates electrons and protons under light, and the LEAT proteins are ectopically expressed in plant cells and interact with the methyl quinones or the derivatives thereof such as plastoquinone to transfer light energy into chemical energy, generate continuous reducing power to change the redox state of plant cells; thereby promoting systematic regulation of Photosystem I (PSI) and Photosystem II (PSII) and increases their efficiency, comprising enhancing ability of light harvesting induced by enhancing the expression level of PSII, PSI and light harvesting complex, enhancing state transition of light energy between the two photosystems, enhancing the transfer efficiency of cyclic and linear electron flow and increasing Rubisco activity, which increase the whole photosynthesis efficiency. In addition, as the fluorescent protein can emit photons at another wavelength which the plant can utilize, the green light and UV are transferred to red light or blue light, thereby expanding the available spectrum range of the plant, increases photosynthesis efficiency of a plant and reduces the damage by UV.

The invention provides a method for improving plant traits, comprising the expression of exogenous LEAT proteins in a plant. The exogenous LEAT proteins form a new synthetic photoreaction with the quinones in a plant which catalyzes water hydrolysis under light, transfers the absorbed light energy into the reducing power stored in quinine molecules with O₂ evolution; and the reduced quinine molecules can participate in a series of oxidation-reduction reactions in a plant, meanwhile, some LEAT proteins are utilized to absorb some rays harmful to the plant, such as UV or blue light with high intensity to protect the photosynthetic apparatus under high light conditions from injures. Thereby the plant photosynthesis is increased, the growth is enhanced and the biomass and yield are increased. The LEAT protein is Blue Fluorescent Protein, Cyan Fluorescent Protein, Green Fluorescent Protein, Yellow Fluorescent Protein, Red Fluorescent Protein or Far-red Fluorescent Protein or the variants thereof.

The methods to make a plant express an exogenous protein are well known in the art. In general, the expression of fluorescent proteins in a plant can be carried out by transformation of the cassette carrying LEAT protein coding genes.

Therefore, the invention further provides a cassette for expressing LEAT proteins in a plant. The expression cassette comprises regulatory elements operable linked with the coding sequence of LEAT proteins, thereby when the cassette is transformed into cells or integrated into genome, the LEAT proteins can be recombinant expressed. The expression cassette comprises a promoter operable linked to the coding sequence of LEAT proteins. The promoter can be any promoter that can drive the expression of the LEAT protein coding sequence in a plant, for example, the promoter can be constitutive (e.g., CaMV35S promoter) or tissue specific or inducible promoters. Under the drive of the promoter, the LEAT protein is expressed and the light utilization of a plant is increased.

In the invention, the expression cassette of LEAT proteins can be inserted into a recombinant expression vector. The term “recombinant expression vector” refers to bacterial plasmid, phage, yeast plasmid, plant cell virus, mammal cell virus or other vectors. In short, any plasmid and vector can be used as long as it can reproduce and be stable in the host.

Furthermore, an expression vector preferably comprises one or more specific label genes to provide phenotype traits for selecting transformed host cells, such as dihydrofolate reductase, neomycin resistance used in eukaryotic cell culture, or kanamycin or ampicillin resistance in E. coli.

As a preferred embodiment of the invention, the method to obtain the plant which expresses LEAT proteins is as follows:

(1) Providing Agrobacterium tumefaciens carrying an expression vector, the expression vector contains the expression cassette of LEAT proteins;

(2) Co-cultivate the plant tissues or organs with the Agrobacterium tumefaciens in step (1), thereby transforming the expression cassette of LEAT proteins into the plant tissues or organs;

(3) Selecting the plant tissues or organs with the expression cassette of LEAT proteins transformed; and

(4) Regenerating the plant tissues or organs in step (3) into a plant.

Wherein, any appropriate mean can be used to perform the method, including reagent, temperature, and pressure condition.

According to the embodiment of the invention, the function of LEAT protein in vitro and in a plant is proved by the following experiments:

(1) The ability of LEAT proteins to catalyze the TMBQ (an analog of plastoquinone) reduction and O₂ evolution under light is proved. It has high activity and the transfer number can be up to 1000 quinone molecules per second. Moreover the function does not depend on whether it has fluorescent characteristic. It is illustrated that these molecules can catalyze water hydrolysis under light to continue to stably provide electrons in the presence of plastoquinone in vivo. And this characteristic generally exists in most of the reported LEAT proteins. Further gene mutation has proved that a protein with very low activity, for example, the activity of GFP is 20 times lower than that of YFP, can obtain very high activity by gene mutation, and reach or surpass the activity of wild type YFP, such as the GFP mutant GFP₁₋₂₃₃ with C-terminal deletion.

(2) mCherry, one of red fluorescent proteins (RFP), is ectopiclly expressed in Brassica and the photosynthesis efficiency of Brassica is detected, the net photosynthetic rate is found to increase 16% under high light conditions (1200 μmol m⁻²s⁻¹) and increase 28% and 31%, respectively under lower light conditions (800 or 400 μmol m⁻²s⁻¹) in the mCherry transgenic Brassica compared with those in the wild type. The mCherry protein has an excitation wavelength of 500 to 610 nm with peak of 587 nm, and an emission wavelength of 560 to 680 nm with peak of 610 nm. As is proved by a series of experiments in vitro, the mCherry protein increases plant photosynthesis by increasing the photosynthesis efficiency, which is mainly due to the enhancement of the systematic adjustment ability of Photosystem I (PSI) and Photosystem II (PSII), including the enhancement of the state transition between the two photosystems, and the enhancement of the efficiency of cyclic and linear electron transfer. Additionally, the mCherry can transfer green light into red light (640 to 665 nm) which can be utilized efficiently by plant Chl a/b in cells, and said red light can be absorbed by chlorophyll, thus increase light energy utilization. Therefore, mCherry transgenic plants are more advantageous under lower light or more green light conditions.

(3) Blue fluorescent proteins (BFP) is ectopicly expressed in Brassica and the photosynthesis efficiency of Brassica is detected, the net photosynthetic rate is found to increase 12.6% and 17.6% respectively under the conditions with light intensity of 1200 μmol m⁻²s⁻¹ and 800 μmol m⁻²s⁻¹, and no significant difference exists under the conditions with light intensity of 400 μmol m⁻²s⁻¹ in the BFP transgenic Brassica compared with those in the wild type. The BFP protein can absorb UVB and UVA at the wavelength of 300 to 400 nm with the peak wavelength of 370 nm, and an emission wavelength of 380 to 500 nm with peak of ˜450 nm, equal to the highest absorption peak of Chl a/b. It is supposed that in addition to the same mechanisms to mCherry, BPF can transfer the UV which possesses the highest energy in sunlight and is harmful to plant cells into blue light which possesses lower energy and can be absorbed by Chl a/b, thereby reducing the harm of UV and increase plant photosynthesis. BFP is mainly used to increase photosynthesis efficiency of a plant under high light or high UV radiation.

(4) mCherry is ectopicly expressed in Brassica and the respiration rate of transgenic etiolated seedling hypocotyl under light is found to be lower than the respiration rate in dark, but no difference exists in the wild type, when the respiration is blocked, net O₂ evolution is observed in the etiolated seedling of transgenic Brassica, indicating that ectopic expression of mCherry in Brassica could establish the system of water hydrolysis and O₂ evolution similar to in vitro reaction with the methyl quinine derivatives in vivo, for example, plastoquinone, etc., thereby the redox state of the transgenic plant cells changes, the reduced materials increase in cells and a more reduced form of cell redox state than the wild type Brassica is produced.

(5) mCherry is ectopicly expressed in Brassica and the transgenic plant is found to grow better than WT, and the fresh weight and dry weight at young seedling stage increase 66.7% and 78%, respectively. The grain yield per plant increases 30 to 100%. The 1000-seed weight increases 25%.

(6) BFP is ectopicly expressed in Brassica and the transgenic plant is found to grow better than WT, and the fresh weight and dry weight increase. The grain yield per plant and 1000-seed weight also increase.

(7) Non-fluorescent or low fluorescent mCherry and YFP mutants are expressed in Brassica and the transgenic plant is found to grow better than WT.

(8) mCherry and BFP are ectopicly expressed in wheat and the transgenic plant is found to grow better than WT, and the seed size and grain yield per plant increase.

(9) mCherry and BFP are ectopicly expressed in rice and the transgenic plant is found to grow better than WT, and the seed size and grain yield per plant increase.

(10) mGFP5, one of green fluorescent proteins (GFP), is ectopicly expressed in rice, wheat and cotton, and the rice, wheat and cotton are found to grow better than WT, and the tiller number, grain yield per plant and 100-seed weight increase, and the number and weight of boll increase. GFP has an excitation wavelength of 395 nm and an emission wavelength of 510 nm. If is supposed that GPF can transfer the UV and blue-violet light in sunlight into green light which possesses lower energy, and thereby reducing the harm of UV and midday high light on plant chloroplast, act as light protection and increase photosynthesis efficiency of a plant under high light. Moreover, though GFP has lower activity to catalyze reduction of methyl quinone and the derivatives thereof, it contributes to some extent under high light.

Those skilled in the art may understand that the mechanism of photosynthesis among plants are very similar, that is: transferring light energy into unstable chemical energy by photoreaction using photosynthetic pigments (mainly chlorophyll, such as Chlorophyll a, Chlorophyll b) and accessory photosynthetic pigments under visible light, and then transferring CO₂ and water into stable organic compounds and releasing O₂ by dark reaction. The key participant of the process is the inner chloroplast which transfers the CO₂ that enters the leaf through stoma and water absorbed by root into starch and releases O₂ under sunlight. Therefore, it is to be understood that the technical solution of the invention is not only applied in Brassica, wheat, rice, and cotton.

### The Main Advantage of the Technical Solution of the Invention is

The method of the invention can expand light energy utilization of a plant and crop and increase photosynthesis efficiency and yield. In the invention, the LEAT protein is transformed into the cytosol of a plant, which regulates the expression of photosynthetic apparatus by changing the redox state of the cytosol, thereby increasing the utilization efficiency of sunlight energy. The method possesses briefness, low cost, good effect, and remarkable result. The invention possesses significance in increasing sunlight utilization of a plant, reducing the harm of harmful radiation (ultraviolet ray UVB) and high light on a plant, thereby increasing photosynthesis efficiency of a plant, finally increasing crop biomass and economic yield. Therefore, the invention can be applied in agriculture, biological energy industries, urban greening, and space life support system, etc.

The invention will be described in further detail below with reference to examples. It is to be understood that the example is merely illustrative and it is not intended that the invention be limited to the example. The experiment methods without describing specific condition in the following experiments are performed of the common condition described in Sambrook J et al., (2002) Molecular cloning: a laboratory manual. 3^{rd} ed. Science Press, or the proposed conditions by the manufacturer. Unless otherwise indicated, all percentages and parts are by weight.

### I. Materials and Methods

### Measurement of Net Photosynthetic Rate

Gas exchange was measured using photosynthetic infrared gas analysis system (LI-6400, Li-Cor Inc, Lincoln, Nebr., USA). The net photosynthetic rate (Pn) and stomatal conductance (Gs) was measured. Sunlight and artificial light were used as artificial light sources for measurement. The ambient CO₂ during the measurements was controlled at 400 μmol mol⁻¹. The ambient temperature was controlled at the temperature for plant growth. Six leaves were measured for each line of plant.

In addition, for the CO₂ response curve of the plant leaf, the light intensity was set at PPFD of 1500 μmol m⁻²s⁻¹ with artificial light source. The Pn under 50-1200 ppm CO₂ was measured.

### Measurement of Millisecond-Delayed Light Emission

Measurements of millisecond-delayed light emission were carried out according to the method described in Chen et al., 2010 (Reversible association of ribulose-1,5-bisphosphate carboxylase/oxygenase activate with the thylakoid membrane depends upon the ATP level and pH in rice without heat stress. Journal of Experimental Botany 61: 2939-2950). Leaf samples with same size were treated in dark for same time and millisecond-delayed light emission was measured in situ. The leaf sample was illuminated with light passing through holes arranged on two rotating wheels, so that the measurement can be partitioned into consecutive cycles of 1 ms excitation by light followed by 4.6 ms darkness. The delayed light emission between 2.8 and 3.8 ms after every flash was recorded. The white light source was supplied by a halogen lamp, and the green light source was obtained by passing the white light source through a 530 nm filter.

### Identification of Transgenic Plants by PCR

Transgenic plants were identified by PCR. Plant genomic DNA was isolated by CTAB method. 200 mg of plant leaves tissue were collected and ground in liquid nitrogen. Then 6 ml of DMA extraction buffer (0.1 M Tris, pH 8.0, 0.5 M NaCl, 0.05 M EDTA, 0.01 M β-mercaptoethanol) and 0.8 ml SDS was added and incubated at 65° C. for 30 min. Then add 2 ml of 5 M KAc, mix and incubate on ice for 30 min. Centrifuge at 4000 rpm, 4° C. for 10 min. Add 6 ml of isopropanol in the supernatant and precipitate for 20 min at room temperature. Centrifuge at 4000 rpm for 10 min and discard the supernatant. Dissolve the pellet in 400 μl H₂O, add 400 μl CTAB buffer (0.2 M Tris, pH 7.5, 0.2 M NaCl, 0.05 M EDTA, 2% CTAB), and incubate at 65° C. for 15 min. Add 800 μl chloroform, mix, and centrifuge at 13000 rpm for 5 min. Transfer the upper aqueous phase to a new centrifuge tube, add 1.4 ml absolute ethanol and precipitate for 15 min. Centrifuge at 13000 rpm for 10 min, discard the supernatant. Wash the pellet with 75% ethanol twice. Dry the pellet and dissolve the pellet in 100 μl H₂O.

2 μl genomic DNA isolated with the method described above was used as template. Target genes mCherry, BFP, mGFP5, YFP or mutant genes were amplified. Primers used for PCR amplification were as follows:

mCherry, BFP, YFP forward primer: 5′-ATGGTGAGCAAGGGCGAGGAG-3′mCherry, BFP, YFP reverse primer: 5′-CTTGTACAGCTCGTCCATGCCG-3′; mGFP5 forward primer: 5′-ATGAGTAAAGGAGAAGAAC-3′; reverse primer: 5′-TTATTTGTATAGTTCATCCAT-3′. PCR program: 95° C. 4 min; 30 cycles of 95° C. 30 sec, 56° C. 30 sec, 72° C. 30 sec, followed by 72° C. 10 min.

### Southern Blot

Southern blot performed with the method described in “Molecular Cloning: A Laboratory Manual”. In detail, 200 μg of genomic DNA from WT or mCherry transgenic Brassica was digested by HindIII, and then transferred onto Hybond N⁺ membrane after electrophoresis. The digoxigenin-labeled λ-DNA digested by HindIII (DNA Molecular weight marker II, Digoxigenin-labeled, Roche, Mannheim, Germany) was used as molecular marker. Southern blot was performed with the method described in “Molecular Cloning: A Laboratory Manual” using digoxingenin labeled full length mCherry as probe. The probe label kit was PCR DIG Probe Synthesis Kit (Roche Applied Science, Mannheim, Germany). The label method was based on the instruction manual. Forward primer: 5′-ATGGTGAGCAAGGGCGAGGAG-3′; reverse primer: 5′-CTTGTACAGCTCGTCCATGCCG-3′.

### RT-PCR

RNA was extracted from leaves with Trizol reagent (Invitrogen, Carlsbad, Calif., USA). 3 μg of total RNA was reversely transcribed with ReverTra Ace reverse transcriptase (Toyobo, Osaka, Japan) in a total volume of 20 μl. For RT-PCR, 2 μl cDNA was used. UBI was used as internal control. Primers for RT-PCR:

mCherry or BFP forward primer:
5′-ATGGTGAGCAAGGGCGAGGAG-3′;
mCherry or BFP reverse primer:
5′-CTTGTACAGCTCGTCCATGCCG-3′;
UBI forward primer:
5′-AGGCCAAGATCCAGGACAAAG-3′;
UBI reverse primer:
5′-CGAGCCAAAGCCATCAAAGAC-3′;

PCR program: For mCherry or BFP amplification, 95° C. for 4 min, 28 cycles of 95° C. for 30 s, 56° C. for 30 s and 72° C. for 30 s, followed by 72° C. for 10 min. UBI was amplified with the same program for 21 cycles.

### Western Blot

Leaves of WT or mCherry transgenic Brassica were ground into fine powder in liquid nitrogen. About 200 mg of plant material were shaked and mixed with the 400 μl extraction buffer (100 mM Tris, pH 7.6, 50 mM NaCl, 5 mM EDTA, 0.2% β-mercaptoethanol, 1% insoluble polyvinyl pyrrolidone and 1 mM phenylmethanesulfonyl fluoride) and centrifuged at 3300×g for 20 min at 4° C. The obtained supernatant was transferred into a new tube and its protein concentration was measured by Bradford method. 50 μg of soluble proteins were separated by 12.5% SDS PAGE and transferred onto PVDF membrane (Milipore, Billerica, Mass., USA). Anti-mCherry antibody (BioVision, San Francisco, Calif., USA) was used as first antibody at 1:1000 dilution. The secondary antibody bovine anti-rabbit IgG-HRP (Santa Craze Biotechnology, Santa Cruz, Calif., USA) was used at 1:10000 dilution. The signal was detected with ECL Western blotting substrate (Pierce, Thermo Scientific, Rockford, Ill., USA) and visualized by exposure to the X-ray film.

### Electron Microscopy and Immunolabeling

Leaves of transgenic Brassica napus L. were preserved by high-pressure freezing/freeze-substitution techniques as described (AndèmeOndzighi et al., Plant Cell 2008, 20(8):2205-20) for immunolabeling. The samples were frozen in Lecia HPM100 and then transferred to liquid nitrogen for storage. Freeze-substitution was performed by put the samples in 0.1% uranyl acetate plus 0.25% glutaraldehyde in acetone in cryo-vials (Nunc, Denmark) for 7 days at −90° C., followed by slow warming to room temperature or −50° C. over a period of one day. After three rinses in acetone, samples were embedded in resin: 33% (24 h), 66% (24 h), and 100% resin (3 days). Polymerization of the resin embedding said material was carried out at −50° C. under UV light for 2 to 3 days in flat bottom embedding capsules. The resin embedded section was placed on the gold net.

For immunolabeling, the sections were blocked with 1% BSA for 30 min, washed with PBS and then incubated with a 10-fold dilution of anti-mCherry antibody (Bio Vision, Cat. 5993-100, San Francisco, Calif., USA) for 2 h at room temperature. Sections were washed again and transferred to a 30-fold dilution of goat anti rabbit IgG-conjugated to 10 nm gold particles (Boshide, Wuhan, China) for 2 h at room temperature. Sections were washed and then stained with uranyl acetate and lead citrate. Observations were performed using a Hitachi H7650 microscope (Hitachi, Japan).

### Measurement of Leaf Absorption Spectrum

Reflectance and transmittance of Brassica leaves under different light intensities were measured using fiber optic spectrometer with an integrating sphere (Ocean Optics, Britain). The absorbance of the leaves under different light intensities was calculated with the equation: absorbance=100%) (without leaf)−transmittance−reflectance.

### Measurements of Chlorophyll Fluorescence and P700 Oxidation-Reduction

Electron transfer efficiency of leaves was measured by chlorophyll fluorescence. The change of chlorophyll fluorescence parameter was monitored with PAM-2000 fluorometer (Walz, Effeltrich, Germany). Electron transfer efficiency of leaves in response to light was measured after 30-min-dark adaption, Fo represents minimum PSII fluorescence after measuring light is turned on. Fm represents maximum PSII fluorescence after a saturating pulse of white light is turned on. The photochemical efficiency of PSII was calculated with the equation: Φ_{PSII}=(Fm′−Fs)/Fm′ (Genty, B., Briantais, J. M., Baker, N. R. (1989). The relationship between the quantum yield of photosynthetic electron transport and quenching of Chl fluorescence. Biochimica et Biophysica Acta, 990, 87-92). The excitation pressure of chlorophyll was calculated with the equation: 1−qL=1−(Fm′−Fs)/(Fm′−Fo)×Fo′/Fs (Kramer, D. M., Johnson, G., Kiirats, O., Edwards, G. E. (2004) New fluorescence parameters for the determination of Q(A) redox state and excitation energy fluxes. Photosynthesis Research, 79, 209-218).

PAM chlorophyll fluorometer with ED-P700 DW-E light absorb unit was used to measure the change of the light absorb at 810-830 nm, then the change of oxidation-reduction state of P700 was obtained. The initial reduction rate of P700 at dark was measured after irradiated under far-red light for 40 s (Klughammer, C et al. (1998), In: Grab, G. (ed) Photosynthesis: mechanisms and effects, Vol 5. Kluwer Academic Publishers, Dordrecht, the Netherlands, 4357-4360; Mi, H. et al. (1992), Plant Cell Physiol. 33: 1099-1105).

### In Vitro Measurements of H₂O Hydrolysis and Oxygen Evolution Catalyzed by LEAT Protein

1.85 ml of 50 mM phosphate buffer (pH6.5) was added in the reaction vessel of Clark-type oxygen electrode, then LEAT proteins (at a final concentration of 0.02-1 μg/ml) and 400 μM 2,3,5-trimethyl-1,4-benzoquinone (TMBQ) or other quinones were sequentially added into the buffer in dark. The oxygen evolution rate was measured under the light with corresponding excitation wave length for LEAT proteins and light intensity at 1-2 μmol m⁻² s⁻¹.

### Measurements of TMBQ Reduction by Fluorescent Proteins Under Light

2 ml of 50 mM PBS buffer (pH6.5) was added into a 4-side-transparent quartz cuvette with volume of 3 ml. Then LEAT proteins at a final concentration of 0.02-1 μg/ml (20 μg/ml for GFP) and 400 μM 2,3,5-trimethyl-1,4-benzoquinone (TMBQ) were sequentially added into the buffer in dark. Decrease in absorption at OD₄₃₆ was measured with UV-3000 (Shimadzu) under the light at a suitable wave length (the light intensity is about 1-2 μmol m⁻²s⁻¹). Extinction coefficient of 41.4 M⁻¹ cm⁻¹ at 436 nm was used to calculate the TMBQ reduction rate.

### Measurement of Leaf State Transition

Leaves were collected from four-week-old grown under white light. State transition of leaves was measured using PAM-101/PDA100 fluorometer (Walz) as described in (L. Dietzel et al., The Plant Cell 23, 2964 (Aug. 1, 2011). PSII light with a PFD of 100 μmol m⁻²s⁻¹ was provided by a Schott KL-1500 lamp (Walz). PSI light with a PFD of 6 μmol m⁻²s⁻¹ was provided by PAM-101 (Walz). The half-time (t_{1/2}) of state 1-state 2 transition was determined by calculate the decrease of the fluorescence after PSI light was switched off. The half-time for state 2-state 1 transition was determined following L. Dietzel et al., The Plant Cell 23, 2964 (Aug. 1, 2011).

### Measurement of State Transition of Plant Thylakoids by 77K Fluorescence

Thylakoids isolated from Brassica leaves were added into STN buffer supplemented with 10 mM ATP in a final concentration of 5-10 μg/ml. Half of thylakoids were illuminated and half of the thylakoids were treated in dark for 20-30 min in room temperature, respectively. Chlorophyll α fluorescence emission spectra were recorded at 77K (in liquid nitrogen) using F-4600 spectrofluorometer (Hitachi, Japan), The excitation wavelength was set at 435 nm. Data were the average of four measurements.

### Measurements of Chlorophyll and Pigments Contents

Chlorophylls and pigments were extracted from Brassica leaves with 80% acetone. Chlorophyll contents were measured as described by Arnon D J 1949, Plant Physiol 24:1-15. Carotenoid contents were measured as described by Niyogi et al., (1997), Proc. Natl. Acad. Sci. U.S.A. 94, 14162-14167.

### Measurement of NAD⁺/NADH, NADP⁺/NADPH, GSSG/GSH and ASC/DHA

Leaves were from the 4-week-old Brassica plants grown under white light or red+blue light, or 2 h after being switched from red+blue light to white light. Extraction of metabolites from leaves and measurement of NAD⁺/NADH, NADP⁺/NADPH, GSSG/GSH and ASC/DHA follows G. Queval, G. Noctor, Anal Biochem. 363, 58 (2007).

### Measurement of Respiration Rate and Oxygen Evolution Rate Under Respiration Depressed Conditions in the Etiolated Hypocotyls

5-6 individual hypocotyls (5-6 cm in length) were collected from 1-week-old etiolated Brassica. The roots and cotyledons were detached and the hypocotyls were cut into 1 mm in length after weighing. The hypocotyls were put into the reaction vessel of the Clack-type oxygen electrode. The changes of dissolved oxygen content in solution over measuring time were recorded in dark or under light, respectively. Then 15 mM HgCl₂ was added to block the respiration, and the change of dissolved oxygen content in solution was recorded. The difference between the respiration rate under light and in dark, and the oxygen evolution rate when respiration was blocked by HgCl₂ was calculated.

### Rubisco Activity Assay

Ribulose-1,5-bisphosphate carboxylase/oxygenase (Rubisco) carboxylase activity assay: Soluble proteins were extracted from fresh leaves with extraction buffer (50 mM Tris-HCl pH 7.8, 1 mM EDTA, 50 mM NaCl, 2 mM β-Mercaptoethanol). The homogenate was centrifuged (12,000 g for 6 min at 4° C.) and the supernatant was used for Rubisco activity assay. Carboxylase activity of Rubisco was measured with ¹⁴C-labelled method as described in (Wang Z Y, Snyder G W, Esau B D, Portis A R, Ogren W L (1992) Species-dependent variation in the interaction of substrate-bound ribulose-1,5-bisphosphate carboxylase/oxygenase (Rubisco) and Rubisco activase. Plant Physiology 100: 1858-1862).

### Gene and Amino Acid Sequences of LEAT Proteins Used

All the LEAT proteins including fluorescent proteins and non-fluorescent mutant proteins used in this invention share similar 3-dimentional cylindrical structure, wherein most of the polypeptide backbone is folded into 11 β-sheets linked with hydrogen bonds, and an alpha helix containing chromophore is in the center. The absorption spectrum covers most of the visible light and part of the ultraviolet light (320-630 nm).

The gene sequence of mCherry is set forth in SEQ ID NO: 1.

The amino acid sequence of mCherry is set forth in SEQ ID NO: 2 (excitation wavelength 480-620 nm).

The gene sequence of BFP is set forth in SEQ ID NO: 3.

The amino acid sequence of BFP is set forth in SEQ ID NO: 4 (excitation wavelength 320-410 nm).

The gene sequence of GFP is set forth in SEQ ID NO: 5.

The amino acid sequence of GFP is set forth in SEQ ID NO: 6 (excitation wavelength 400-510 nm).

The gene sequence of YFP is set forth in SEQ ID NO: 7.

The amino acid sequence of YFP is set forth in SEQ ID NO: 8 (excitation wavelength 450-530 nm).

The gene sequence of CFP is set forth in SEQ ID NO: 9.

The amino acid sequence of CFP is set forth in SEQ ID NO: 10 (excitation wavelength 350-490 nm).

Gene and amino acid sequences of YFP mutants:

The gene sequence of YFPmu2 (YFP^{H149C Y204A}) is set forth in SEQ ID NO: 11.

The amino acid sequence of YFPmu2 (YFP^{H149C Y204A}) is set forth in SEQ ID NO: 12.

The gene sequence of YFPmu4 (YFP^{H149C F166N I168M Y204A}) is set forth in SEQ ID NO: 13.

The amino acid sequence of YFPmu4 (YFP^{H149C F166N I168M Y204A}) is set forth in SEQ ID NO: 14.

The gene sequence of YFPmu7 (YFP^{S148C H149C F166N K167M I168M S203A Y204A}) is set forth in SEQ ID NO: 15.

The ammo acid sequence of YFPmu7 (YFP^{S148C H149C F166N K167M I168M S203A Y204A}) is set forth in SEQ ID NO: 16.

The gene sequence of YFP^{L232H} is set forth in SEQ ID NO: 17.

The amino acid sequence of YFP^{L232H} is set forth in SEQ ID NO: 18.

The gene sequence of YFP^{L232Q} is set forth in SEQ ID NO: 19.

The amino acid sequence of YFP^{L232Q} is set forth in SEQ ID NO: 20.

Gene and amino acid sequences of mCherry mutants:

The gene sequence of mCherrymu3 (mCherry^{S151C S152C K167M}) is set forth in SEQ ID NO: 21.

The amino acid sequence of mCherrymu3 (mCherry^{S151C S152C K167M}) is set forth in SEQ ID NO: 22.

The gene sequence of mCherrymu4 (mCherry^{S151C S152C K167M I202A}) is set forth in SEQ ID NO: 23.

The amino acid sequence of mCherrymu4 (mCherry^{S151C S152C K167M I202A}) is set forth in SEQ ID NO: 24.

The gene sequence of mCherrymu5 (mCherry^{S151C S152C H166N K167M I202A}) is set forth in SEQ ID NO: 25.

The amino acid sequence of mCherrymu5 (mCherry^{S151C S152C H66N K167M I202A}) is set forth in SEQ ID NO: 26.

The gene sequence of mGFP5 is set forth in SEQ ID NO: 27.

The amino acid sequence of mGFP5 is set forth in SEQ ID NO: 28.

The gene sequence of eqFP611 (AY130757) is set forth in SEQ ID NO: 29.

The amino acid sequence of eqFP611 is set forth in SEQ ID NO: 30.

The gene sequence of hcriCP (AF363776) is set forth in SEQ ID NO: 31.

The amino acid sequence of hcriCP is set forth in SEQ ID NO: 32.

The gene sequence of eforCP (EU498726) is set forth in SEQ ID NO: 33.

The amino acid sequence of eforCP is set forth in SEQ ID NO: 34.

The gene sequence of efasCFP (DQ206397) is set forth in SEQ ID NO: 35.

The amino acid sequence of efasCFP is set forth in SEQ ID NO: 36.

The gene sequence of spisCP (DQ206398) is set forth in SEQ ID NO: 37.

The amino acid sequence of spisCP is set forth in SEQ ID NO: 38.

The gene sequence of scubGFP (AY037767) is set forth in SEQ ID NO: 39.

The amino acid sequence of scubGFP is set forth in SEQ ID NO: 40.

The gene sequence of rfloRFP (AY037773) is set forth in SEQ ID NO: 41.

The amino acid sequence of rfloRFP is set forth in SEQ ID NO: 42.

The gene sequence of rmueGFP (AY015996) is set forth in SEQ ID NO: 43.

The amino acid sequence of rmueGFP is set forth in SEQ ID NO: 44.

The gene sequence of ceriantRFP (AY296063) is set forth in SEQ ID NO: 45.

The amino acid sequence of ceriantRFP is set forth in SEQ ID NO: 46.

The gene sequence of anm2CP (AY485336) is set forth in SEQ ID NO: 47.

The amino acid sequence of anm2CP is set forth in SEQ ID NO: 48.

The gene sequence of phiYFP (AY485333) is set forth in SEQ ID NO: 49.

The amino acid sequence of phiYFP is set forth in SEQ ID NO: 50.

The gene sequence of cpGFP (AB185173) is set forth in SEQ ID NO: 51.

The amino acid sequence of cpGFP is set forth in SEQ ID NO: 52.

The gene sequence of YFP₁₋₂₃₁ is set forth in SEQ ID NO: 61.

The amino acid sequence of YFP₁₋₂₃₁ is set forth in SEQ ID NO: 62.

Gene and amino acid sequences of GFP mutants:

The gene sequence of GFP₁₋₂₃₁ is set forth in SEQ ID NO: 63.

The amino acid sequence of GFP₁₋₂₃₁ is set forth in SEQ ID NO: 64.

### Plasmid Construction for Plant Transformation

mCherry, mCherrymu3, mCherrymu4, mCherrymu5, BFP, YFPmu7 and mGFP5 genes were amplified by PCR and cloned into vector pHB (Mao et al., PNAS_Aug. 23, 2005_vol.102_no. 34_12270-12275; http://www.pnas.org/content/102/34/12270/suppl/DCl#F7) with the restriction sites of HindIII/XbaI. The obtained plasmids carrying corresponding genes were used for plant transformation. The map of pHB was shown in FIG. 13A.

For of pHB-mCherry construction, pmCherry vector (Clontech, Mountain View, Calif., USA) was used as template for PCR amplification with forward primer: 5′-CCCAAGCTTATGGTGAGCAAGGGCGAGGAG-3′ (SEQ ID NO: 53) and reverse primer: 5′-CCGTCTAGACTACTTGTACAGCTCGTCCATG-3′ (SEQ ID NO: 54). The restriction sites of HindIII and XbaI in the forward and reverse primers were underlined. PCR reaction was 95° C. 4min; 30 cycles for 95° C. 30 sec, 56° C. 30 sec, 72° C. 30 sec; followed by 72° C. for 10 min. PCR product was digested with HindIII/XbaI and ligated to the pHB vector digested with HindIII/XbaI.

For pHB-RFP construction, pRSET-BFP (Invitiogen, Cat# v354-20) was used as template for PCR amplification with forward primer: 5′-CCCAAGCTTATGGTGAGCAAGGGCGAGGAG-3′ (SEQ ID NO: 55) and reverse primer: 5′-CCGTCTAGATTACTTGTACAGCTCGTCCATG-3′ (SEQ ID NO: 56). The restriction sites of HindIII and XbaI in the forward and reverse primers were underlined. PCR reaction was performed as for mCherry amplification. PCR product was digested with HindIII/XbaI and ligated to the pHB vector digested with HindIII/XbaI.

mCherrymu3, mCherrymu4, mCherrymu5 and YFPmu7 genes were obtained by DNA synthesis (Geneseript, Nanjing, China). The synthesized mCherrymu3, mCherrymu4 and mCherrymu5 fragments were cloned into vector pUC57 with restriction sites BamHI/SacI. YFPmu7 fragment was cloned into vector pUC57 with restriction sites KpnI/SacI. For pHB-mCherrymu3, pHB-mCherrymu4 and pHB-mCherrymu5 construction, pUC57 -mCherrymu3, pUC57-mCherrymu4 and pUC57-mCherrymu5 were used as templates for PCR amplification, respectively. For pHB-YFPmu7 construction pUC57-YFPmu7 was used as template for PCR amplification. PCR primers and reactions were same as for mCherry amplification. PCR products were digested with HindIII/XbaI and ligated to the pHB vector digested with HindIII/XbaI. Map of pUC 57 was shown in FIG. 12.

For pHB-mGFP5 construction, pCambia-1302 (Cambia) was used as template for PCR amplification using forward primer: 5′-CCCAAGCTTATGAGTAAAGGAGAAGAAC-3′ (SEQ ID NO: 57) and reverse primer: 5′-CCGTCTAGATTATTTGTATAGTTCATCCAT-3′ (SEQ ID NO: 58). The restriction sites of HindIII and XbaI in the forward and reverse primers were underlined. PCR reaction was performed as for mCherry amplification. PCR product was digested with HindIII/XbaI and ligated to the pHB vector digested with HindIII/XbaI.

### Plant Transformation

Brassica, wheat, rice and cotton were transformed by Agrohacteria infection method.

Rice transformation was performed with method described in Liu Qiaoquan, Zhang Jingliu, Wang Zongyang, Hong Mengming and Gu Minghong (1998). A highly efficient transformation system mediated by Argrobacteriun tumefaciens in rice (Oryza sativa L.). Acta Phytophysiologica Sinica 24, 259-271.

Brassica transformation was performed with method described in Zhang H X, Hodson J N, Williams J P, and Blumwald E. (2001) Engineering salt-tolerant Brassica plants: Characterization of yield and seed oil quality in transgenic plants with increased vacuolar sodium accumulation. Proceeding of National Academy of Science USA 98, 12832-12836.

Wheat transformation was performed with method described in Supartana P, Shimizu T, Nogawa M, Shioiri H, Nakajima T, Haramoto N, Nozue M, and Kojima M. Development of simple and efficient in Planta transformation method for wheat (Triticum aestivum L.) Using Agrobacterium tumefaciens, Journal of Bioscience and Bioengineering 102, 162-170.

Cotton transformation was performed with method described in Yue Jianxiong, Zhang Huijun and Zhang Lianhui (2002). Hygromycin resistance as an efficient selectable marker for cotton stable transformation. Cotton Science, 14(4), 195-199.

As described above, transgenic Brassica, transgenic wheat, transgenic rice and transgenic cotton were obtained.

### Prokaryotic Expression and Purification of LEAT Proteins

Full length of mCherry gene was amplified by PCR. PCR product was digested with BamHI and SalI and cloned into pGEX-4T-1 (GE healthcare, Uppsala, Sweden) to make the GST fused at the N-terminal of mCherry. The plasmid was transformed into E. coli BL21 (DE3) (Promega, Madison, Wis.). Forward primer 5′-CCCGGATCCATGGTGAGCAAGGGCGAGGAG-3′ (SEQ ID NO: 59) and reverse primer: 5′-CCGGTCGACCTACTTGTACAGCTCGTCCATG-3′ (SEQ ID NO: 60) was used for PCR amplification. The restriction sites of BamHI and SalI in the forward and reverse primers were underlined.

pRSET-BFP was digested with EcoRI/XhoI and full length of BFP gene fragment was ligated with pGEX-4T-1 digested with EcoRI/XhoI to make the GST fused at the N-terminal of BFP. The plasmid was transformed into E. coli BL21 (DE3).

mCherry mutant genes (mCherrymu3, mCherrymu4 and mCherrymu5) were synthesized (Genescript, Nanjing, China) with BamHI and SacI restriction sites at both sides of the genes. The synthesized mCherrymu3, mCherrymu4 and mCherrymu5 genes were ligated into pUC57 at the restriction sites BamHI/SacI. The pUC57-mCherrymu3, pUC57-mCherrymu4 and pUC57-mCherrymu5 were digested with BamHI and SacI, and the fragments were ligated with pET30a (Novagen) to make the 6×His tag fused at the N-terminal of mCherry mutant genes. The plasmids were transformed into E. coli BL21 (DE3) to induce the expression of recombinant proteins.

YFP, CFP, GFP genes as well as C-terminal truncated YFP (YFP₁₋₂₃₁) and GFP mutant (GFP₁₋₂₃₁) were amplified by PCR with iProof High-Fidelity DNA polymerase (Bio-rad) using pEYFP, pECFP (Clonetech) and p1301-GFP (Li N, Zhang D-S, Liu H-S et al. The rice tapetum degeneration retardation gene is required for tapetum degradation and anther development. The Plant Cell 2006; 18: 2999-3014. ) as templates, respectively. The PCR products were digested with KpnI and SacI and ligated with pET51b vector (Novagen). YFPmu2 and YFPmu4 mutation sites were obtained by PCR mutagenesis. All the genes above were fused with strep II at the N-terminal. The plasmids were sequenced and the plasmids with the corrected sequences were transformed into E. coli BL21-CodonPlus strain (Progema, Madison, Wis.). The recombinant proteins were induced with 0.1 ml IPTG over night at 20° C.

Twelve LEAT genes were obtained by gene synthesis (Genescript, Nanjing, China). The LEAT genes were synthesized with restriction sites BamHI and SacI or EcoRI and SacI at both sides of the genes. The synthesized genes were digested with BamHI and SacI or EcoRI and SacI and ligated with pET30a (Novagen) to make the 6×His tag fused at the N-terminal of the genes. The plasmids were transformed into E. coli BL21 (DE3) to induce the expression of recombinant proteins.

The above all recombinant proteins were induced according to the user manual provided by the manufacture. The purified proteins were desalt and stored in 50 mM PBS buffer supplemented with 10% glycerol at −80° C.

### II. EXAMPLES

### Example 1

### LEAT Proteins can Utilize the Light Energy and Catalyze the H₂O Hydrolysis

### 1. LEAT Protein can Catalyze the Analogue of Plastoquinone 2,35-trimethyl-1,4-benzoquinone Reduction Under Light

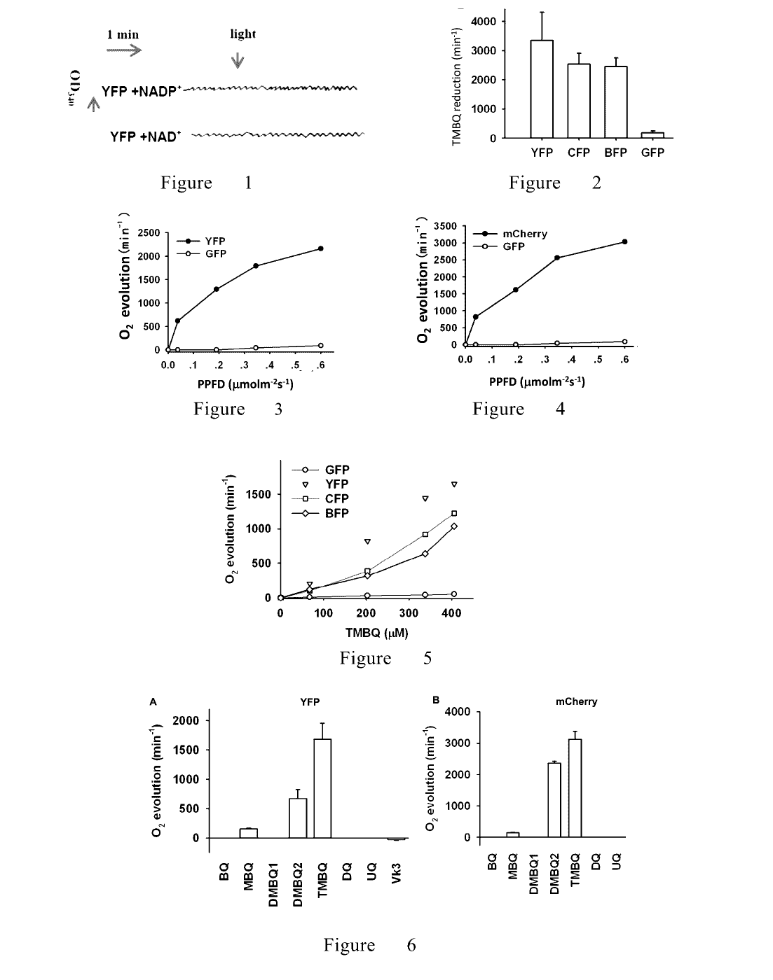

It was reported that proteins such as GFP can function as electron donor to reduce the oxidized small compounds such as NAD⁺, potassium ferricyanide, and some proteins such as cytochrome c, and FAD-containing proteins (Bogdanov A. M. et al, 2009, Nature Chemical Biology. 5:459-461), The invertors used E. coli to express and purify recombinant proteins. Although pre-illuminated YFP and mCherry can function as electron donors, 1 LEAT protein only can provide 1 electron, and therefore cannot continuously provide electrons under light (FIG. 1). Quinone reduction under light was analyzed by monitoring the decrease of absorption at 436 nm.

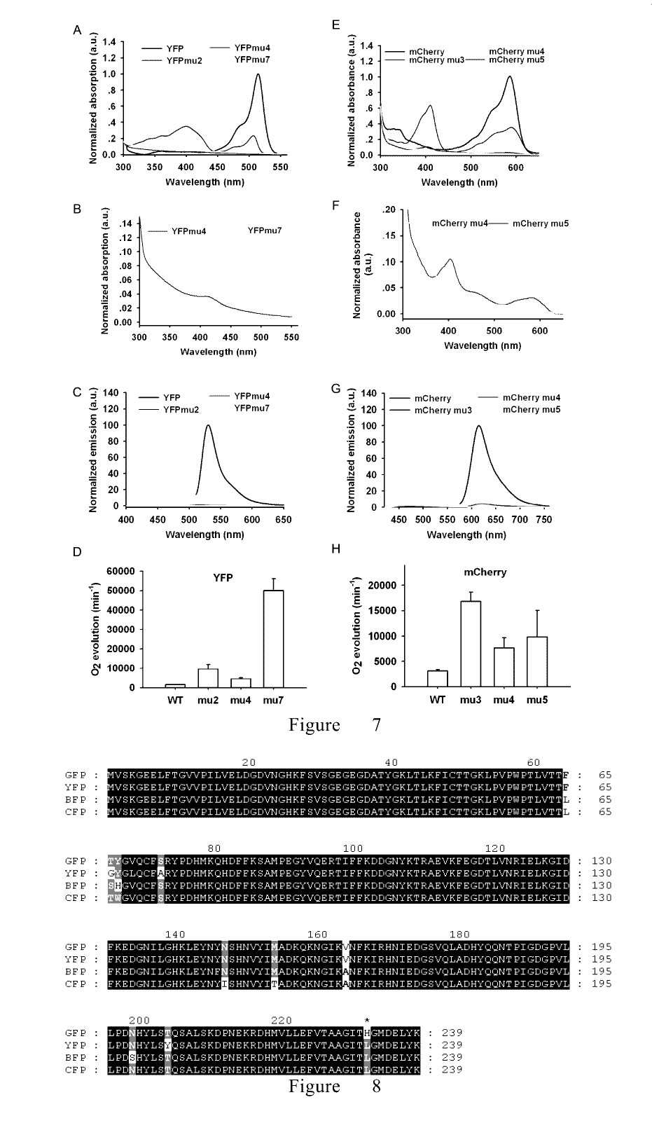

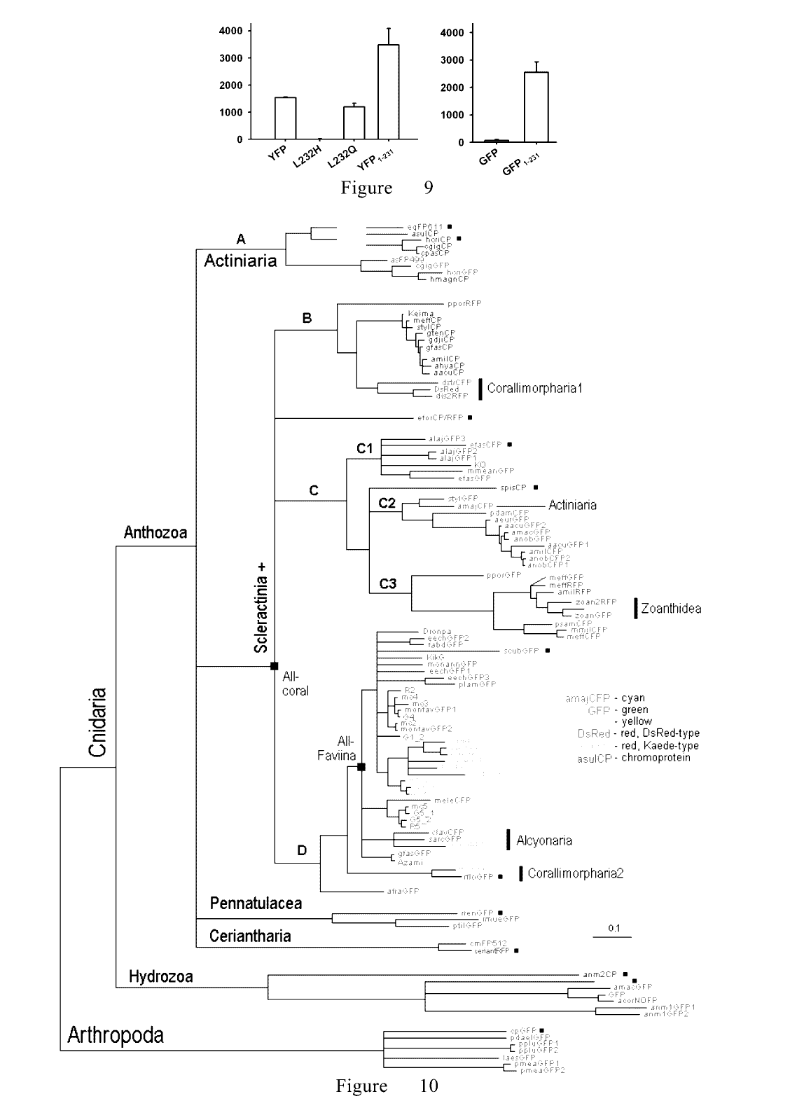

LEAT protein can function as a kind of pigments like Chl a in the reaction center, catalyze the the reduction of methyl quinones and their variants, such as the analog of plastoquinone, 2,3,5-trimethyl-1,4-benzoquinone by water (FIG. 2). Just like the reduction of TMBQ under light, the O₂ evolution catalyzed by LEAT protein is dependent on the light intensity. Although GFP also can catalyzed the O₂ evolution from water, its activity is much lower (FIGS. 3 and 4). All the other LEAT proteins have strong activities on catalyzing the reduction of TMBQ and O₂ evolution, which are depended on the TMBQ concentration (FIG. 5).

YFP/mCherry also can catalyze the hydrolysis of water under light in the presence of other methyl quinones. In the presence of TMBQ, water hydrolysis and O₂ evolution activity of YFP and mCherry is the highest. Among the quinones tested, the activity of water hydrolysis by YFP and mCherry protein is TMBQ>DMBQ2>MBQ>DMBQ1. When TMBQ is substituted with Duro quinone (DQ, tetramethyl-1,4-benzoquinone) or a analog of ubiquinone (UQ, 2,3-dimethoxyl-5-methyl-1,4-benzoquinone), YFP and mCherry does not have water photohydrolysis and O₂ evolution activity under light (FIG. 6). There are various types of quinone exist in plant, among which methyl quinine derivative is one of the most important quinone, i.e. PQ, which is the analog of TMBQ, exists widely in cytosol and chloroplast in high abundant.

### 2. The Activity of LEAT on TMBQ Reduction, Water Photohydrolysis and O₂ Evolution is not Dependent on the Fluorescence Ability

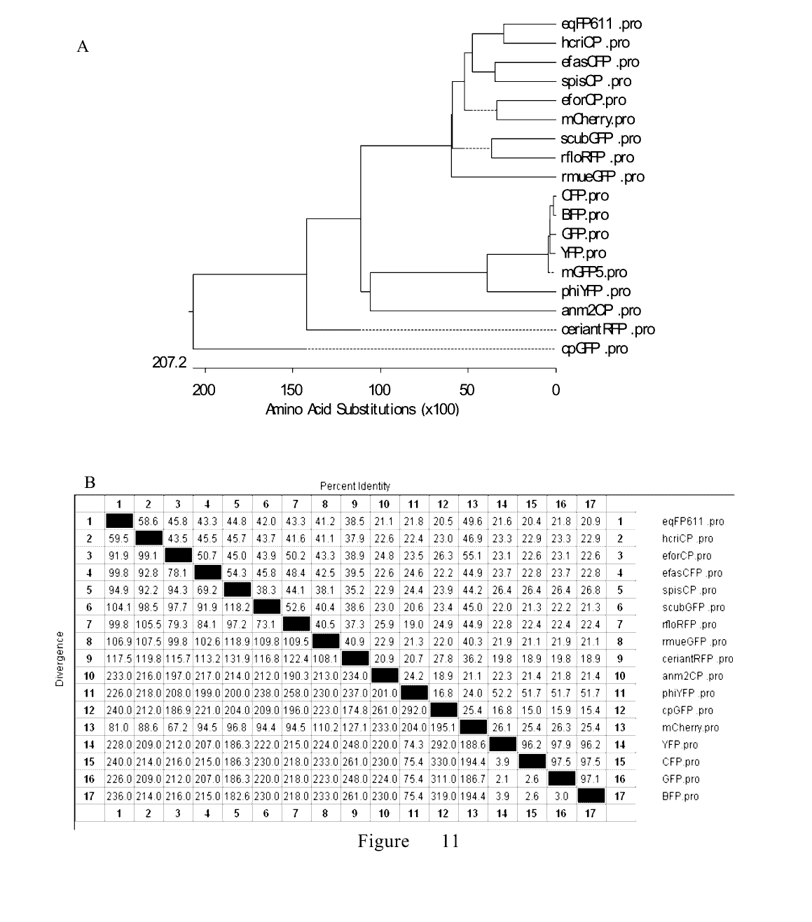

In nature there are a lot of chromphore proteins, which cannot emit fluorescence but can absorb the light. To further explore the relationship between the fluorescence of LEAT protein and its activity on water photohydrolysis and O₂ evolution, the inventor mutated some amino residues around the chromophore triplet of YFP and mCherry, and obtained 3 mutants for YFP and mCherry, respectively, with decreased fluorescence. These mutants are: YFPmu2(YFP^{H149CY204A}), YFPmu4(YFP^{H149CF166NI168MY204A}), YFPmu7(YFP^{S148CH149CF166NK167MI168MS203AY204A}), mCherrymu3(mCherry^{S151CS152CK167M}), mCherrymu4 (mCherry^{S151CS152CK167MI202A}) and mCherrymu5 (mCherry^{S151CS152CH166NNK167MI202A}). The inverters compared the absorbance, fluorescence, and the activities of TMBQ reduction, water hydrolysis and O₂ evolution under light. Results of absorbance and fluorescence spectra analysis showed that only very low or almost no fluorescence emit from these 6 mutants. Among them, the absorbance of YFPmu2 and mCherrymu3 decreased to 30% and 40%) of their original proteins (FIGS. 7A and 7E), and their fluorescence decreased to 1% and 4% of their original proteins (FIGS. 7B and 7D). Both fluorescence and absorbance decreased much more in other 4 mutants. Compare with WT YFP, the fluorescence of YFPmu4 and YFPmu7decreased to 0.04% and 0.07% (FIGS. 7A, 7B, 7E and 7F), and fluorescence of mCherrymu4 and mCherrymu5 was only about 3% and 5% to that of WT (FIGS. 7C and 7G).

Further analysis of water hydrolysis and O₂ evolution under light showed that LEAT proteins catalyze quinone reduction and O₂ evolution independent of their fluorescence. The activity of O₂ evolution is not decreased along with the decrease of fluorescence, on the contrary, the activity is markedly increased, wherein the O₂ evolution rate catalyzed by YFPmu2 and YFPmu7 increased for 6 and 30 folds, respectively; and O₂ evolution rate catalyzed by mCherrymu3 increased for 6 folds (FIGS. 7D and 7H).These results suggested that O₂ evolution catalyzed by LEAT protein via quinone is not depend on their fluorescence, but depend on their capacity of light energy absorption, transduction and conversion. Because the energy was not dissipated through fluorescence their activities of TMBQ reduction and O₂ evolution were much higher than those of control.

### 3. The O₂ Evolution Activity of YFP Mutants: YFP^{L232H}, YFP^{L232Q}, YFP₁₋₂₃₁ and GFP₁₋₂₃₁

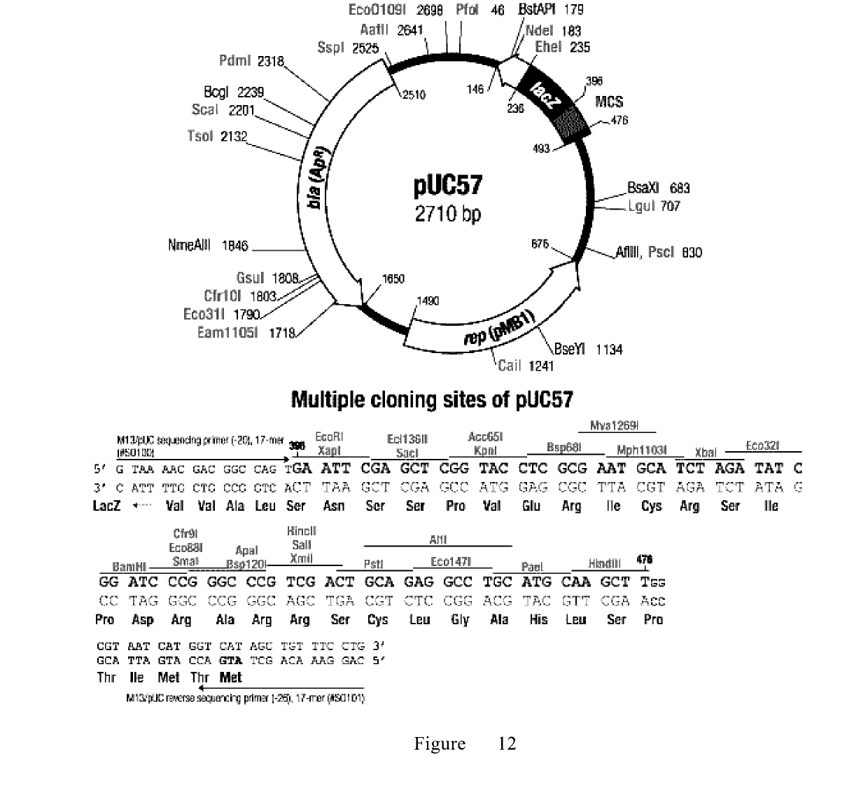

By comparing the amino acid sequences of GFP, YFP, CFP and BFP (FIG. 8), it was found that the 232nd residue may influence the O₂ evolution activity. The O₂ evolution activity of YFP^{L232H} mutant decreased to the level that less than 1% of the wild type YFP (FIG. 9A). However, the O₂ evolution activity by catalyzing the water hydrolysis in the presence of TMBQ was very high by deleting the C-terminals of YFP (FIG. 9A) and GFP (FIG. 9B). This result suggested that low activity of GFP is relate to His232, moreover, LEAT proteins with low activity can be engineered simply, such as truncating the C-terminal, to increase their activity greatly, and be used in the improvement of photosynthesis efficiency of the plant.

### Example 2

### Comparison of the Water Hydrolysis Activities Under Light of LEATs with Different Origins

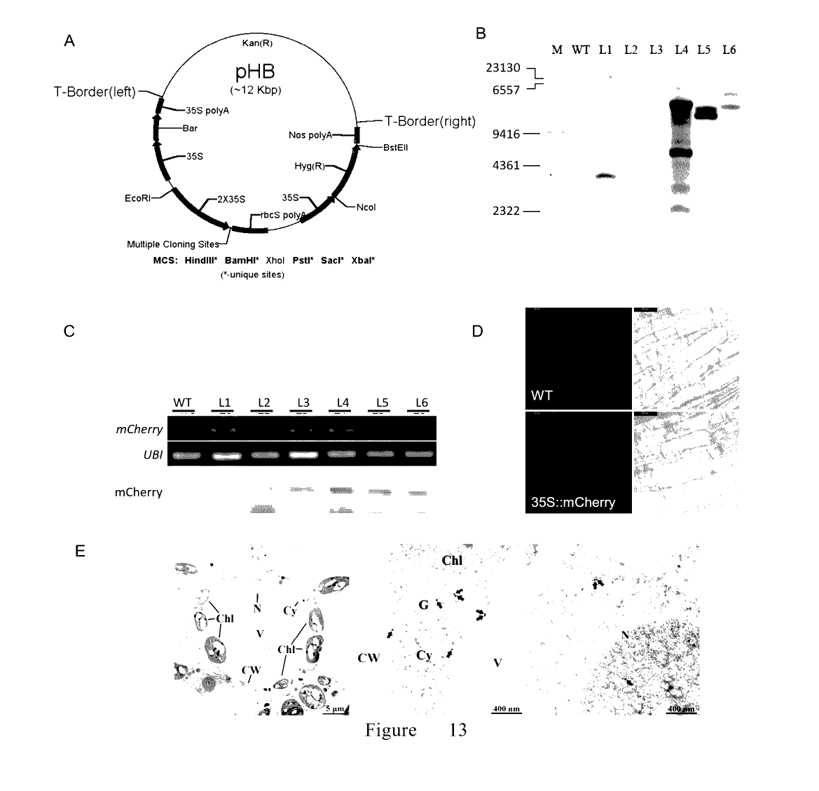

Among 110 fluorescent and chromophore proteins from Cnidarian and Arthropoda (Table 1), 9 fluorescent proteins and 3 chromophore proteins were selected (FIG. 10). These 12 proteins are belong to A, B, C, D and other different limbs of the phylogenetic tree (FIG. 10). These 12 proteins together with GFP and dsRed series of fluorescent proteins cover almost all different types of fluorescent and chromophore proteins reported (Alieva et al., 2008, Diversity and evolution of coarl fluorescent proteins. PLoS One 3(7): e2680, doi:10.1371/journal.pone.0002680). Big differences exist between the 12 proteins and GFP or dsRED series of fluorescent proteins in molecular evolution and amino acid sequence homology (FIGS. 11A and 11B). Although the intensify of fluorescence, absorbance and fluorescence spectra of these recombinant proteins expressed in E. coli are different, they all showed activities of O₂ evolution to a different degree (Table 2). It is possible to use these proteins or the mutants thereof for improving the phenotype of plants by utilizing their abilities of supplying electrons continuously from water hydrolysis under light after suitable genetic engineering.

TABLE 1
The gene ID and accession numbers of 110 fluorescent and chromophore
proteins from Cnidarian and Arthropoda (Alieva et al., 2008).

| | | | | | | Chromophore |
|---|---|---|---|---|---|---|
| | | | | DsRed type of | Kaede type of | protein without |
| CFP | GFP | | | RFP | RFP | fluorescence |
| amajCFP | aacuGFP1 | eechGFP3 | pdaelGFP | amilRFP | cjarRFP | aacuCP |
| AF168421 | AY646069 | DQ206396 | AY268076 | AY646073 | EF186664 | AY646077 |
| amilCFP | aacuGFP2 | efasGFP | pmeaGFP1 | ceriantRFP | dendRFP | ahyaCP |
| AY646070 | AY646066 | DQ206385 | AY268074 | AY296063 | AF420591 | AY646076 |
| anobCFP1 | acorNOFP | fabdGFP | pmeaGFP2 | dis2RFP | eechRFP | anm2CP |
| AY646072 | AY151052 | EU498723 | AY268075 | AF272711 | DQ206387 | AY485336 |
| anobCFP2 | aeurGFP | KikG | ppluGFP1 | eqFP611 | G1_1 | amilCP |
| AY646071 | EU498722 | AB193294 | AY268071 | AY130757 | AY182019 | AY646075 |
| clavCFP | afraGFP | G1_2 | ppluGFP2 | KO AB128820 | EosFP | asulCP |
| AF168424 | AY647156 | AY182020 | AY268072 | | AY765217 | EF587182 |
| dstrCFP | alajGFP1 | G4 | pporGFP | meffRFP | Kaede | cgigCP |
| AF168420 | AY508123 | AY182021 | DQ206391 | DQ206379 | AB085641 | AF363775 |
| efasCFP | alajGFP2 | Azami | ptilGFP | pporRFP | mc1 | cpasCP |
| DQ206397 | AY508124 | AB107915 | AY015995 | DQ206380 | AY181552 | AF383155 |
| G5_1 | alajGFP3 | gfasGFP | rfloGFP | DsRed AF168419 | meleRFP | gfasCP |
| AY182022 | AY508125 | DQ206389 | AY037772 | | DQ206386 | DQ206394 |
| G5_2 | amacGFP | GFP | rmueGFP | zoan2RFP | R1_2 | gdjiCP |
| AY182023 | AF435432 | P42212 | AY015996 | AY059642 | AY182013 | DQ206376 |
| meffCFP | amilGFP | hcriGFPAF | R2 | | rfloRFP | gtenCP |
| DQ206381 | AY646067 | 420592 | AY182014 | | AY037773 | AF38315 |
| mmilCFP | anm1GFP1 | laesGFP | rrenGFP | | scubRFP | hcriCP |
| DQ206392 | AY485334 | AY268073 | AF372525 | | AY646064 | AF363776 |
| meleCFP | anm1GFP2 | mc2 | sarcGFP | | | hmagnCP |
| DQ206382 | AY485335 | AY181553 | EU498725 | | | AY461714 |
| mc5 | anobGFP | mc3 | scubGFP | | YFP | Keima |
| AY181556 | AY646068 | AY181554 | AY037767 | | | AB209967 |
| pdamCFP | asFP499 | mc4 | stylGFP | | phiYFP | meffCP |
| AY679113 | AF545827 | AY181555 | DQ206390 | | AY485333 | DQ206377 |
| psamCFP | cmFP512 | meffGFP | zoanGFP | | zoanYFP | stylCP |
| EU498721 | AF545830 | DQ206393 | AF168422 | | AF168423 | DQ206378 |
| R5 | cgigGFP | mmeanGFP | | | | spisCP |
| AY182017 | AY037776 | AY155344 | | | | DQ206398 |
| | cpGFP | monannGFP | | | Chromo-Red | |
| | AB185173 | AY037766 | | | | |
| | Dronpa | monfavGFP1 | | | eforCP/RFP | |
| | AB180726 | AY679112 | | | EU498726 | |
| | eechGFP1 | monfavGFP2 | | | | |
| | DQ206383 | AF401282 | | | | |
| | eechGFP2 | plamGFP | | | | |
| | DQ206395 | EU498724 | | | | |

Note:
The proteins underlined were expressed in E. coil and purified, and the water hydrolysis and O₂ evolution activities under light were investigated.

TABLE 2
Comparison the fluorescence intensity of different LEAT proteins and
their activities in water hydrolysis and O₂ evolution under light.

| | | | Excitation | | |
|---|---|---|---|---|---|
| | LEAT protein | | wavelength | | O₂ evolution |
| No. | (GenBank accession No.) | Protein source | (nm) | Fluorescence | activity |
| 1 | eqFP611 (AY130757) | Entacmaea quadricolor | 520-630 | weak | + |
| 2 | hcriCP (AF363776) | Heteractis crispa | 520-620 | Emission | + |
| | | | | wavelength: | |
| | | | | 795-815 | |
| | | | | nm, >850 nm | |
| | | | | Infrared ray | |
| 3 | efofCP/RFP (EU498726) | Echinopora forskaliana | 510-610 | Strong | + |
| 4 | efasCFP (DQ206397) | Eusmilia fastigiata | 400-480 | Strong | + |
| 5 | spisCP (DQ206398) | Stylophora pistillata | 460-510 | Extream weak | ++ |
| 6 | scubGFP (AY037767) | Scolymia cubensis | 450-530 | Strong | + |
| 7 | rfloRFP (AY037773) | Ricordea florida | 440-515 | weak | ++ |
| 8 | rmueGFP (AY015996) | Renilla muelleri | 420-470 | weak | ++ |
| 9 | ceriantRFP (AY296063) | Cerianthus sp. DW-2003 | 470-595 | strong | ++ |
| 10 | anm2CP (AY485336) | Anthomedusae sp. SL-2003 | 490-590 | Emission | + |
| | | | | wavelength: | |
| | | | | 830-870 nm, | |
| | | | | Infrared ray | |
| 11 | phiYFP (AY485333) | Phialidium sp. SL-2003 | 470-545 | Strong | ++ |
| 12 | cpGFP (AB185173) | Chiridius poppei | 420-525 | Stong | + |
| 13 | GFP series | Aequorea Victoria | | | |
| | GFP | | 400-510 | strong | low |
| | BFP | | 330-410 | strong | + |
| | YFP | | 450-530 | strong | + |
| | CFP | | 350-490 | Strong | + |
| 14 | mCherry | Discosoma sp | 300-400 | Srong | + |
| | | | 480-620 | | |

Note:
In the O₂ evolution assays, “+”: similar O₂ evolution activity as that of YFP; “++”: higher O₂ evolution activity than that of YFP by at least one order of magnitude; “weak”: lower activity than that of YFP by at least one order of magnitude.

### Example 3

### Study on LEAT Protein (mCherry) Transgenic Plants

### 1. Expression of mCherry in Brassica Increased Net Photosynthesis Rate, Plant Growth and Biomass of the Transgenic Plant

The mCherry transgenic Brassica was confirmed. The results of Southern blot analysis of mCherry transgenic Brassica was shown in FIG. 13B. The fluorescence signal of mCherry expressing in the root cells of transgenic Brassica was observed (FIG. 13C). Results of RT-PCR and Western blot analysis of transgenic plant line (L1-L6) were shown in FIG. 13D. The mCherry protein localization in plant cell was analyzed by electron microscopy and immunolabeling as shown in FIG. 13E. The above results suggested that L1-L6 are positive transgenic mCherry lines, and mCherry protein was expressed in cytosol (Cy) and nucleus (N) of the cell. In this invention, T2 generation of mCherry transgenic Brassica was used for the experiments.

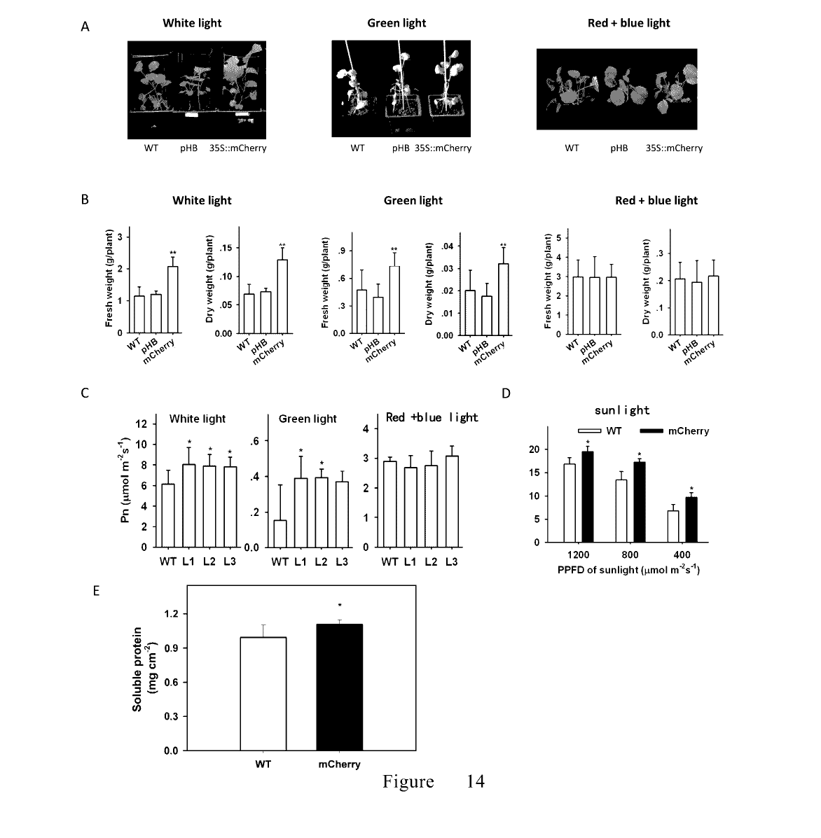

In this invention, three lines of mCherry transgenic Brassica were selected and the content of soluble protein was measured and compared with that in WT. The result was shown in FIG. 14E. The content of soluble protein in mCherry transgenic Brassica was increased.

Wild type (WT), transgenic Brassica with empty vector (pHB) and mCherry transgenic Brassica (L1-L3) were germinated in the phytotron under white light (light intensity: 250 μmol m⁻²s⁻¹) for 1 week. Then the plants were transferred and continued to grow under white light, green light (light intensity: 60 μmol m⁻²s⁻¹), red+blue light (intensity of red light: 60 μmol m⁻²s⁻¹; intensity of blue light: 10 μmol m⁻²s⁻¹). The paragraphs were taken three weeks after transfer. As shown in FIG. 14A, mCherry transgenic Brassica grew better than WT and empty vector control plants when the excitation light of mCherry i.e. white light and green light are presented. No significant difference was observed when excitation light was absent, i.e. under red+blue light.

FIG. 14B showed the comparison of fresh and dry weights of 4-week-old wild type (WT) and mCherry transgenic Brassica grown under white light, green light and red+blue light. The result suggested that the fresh and dry weights of mCherry transgenic Brassica were significantly increased as compared to those of WT and empty vector control plants under white or green light, however, no significant difference was observed under red+blue light.

Net photosynthesis rate of WT and mCherry transgenic Brassica under different light qualities. WT and mCherry transgenic Brassica (L1, L2 and L3) were grown in the phytotron (light intensity: 250 μmol m⁻²s⁻¹) for 9 weeks. Then the plants were transferred to different light qualities for 4 days. The net photosynthesis rate was measured. The results in FIG. 14C showed that net photosynthesis rate of mCherry transgenic Brassica was significantly increased under white light and green light.

Net photosynthesis rate of WT and mCherry transgenic Brassica grown in the natural field conditions were measured under different light intensities. Results showed in FIG. 14D suggested that the net photosynthesis of mCherry transgenic Brassica culture under nature light was significantly increased than that of WT and transgenic Brassica with empty vector, especially when measured under lower light intensity.

FIG. 15A showed the fresh and dry weights per plant of WT and mCherry transgenic Brassica (L1-L6 represented 6 different transgenic lines). FIG. 15B showed comparison of the size of plant, silique and seeds, grain yield per plant and 1000-seed weight between WT and mCherry transgenic Brassica at the harvesting time. Therefore, expression of mCherry in Brassica stimulated the growth and biomass accumulation in the natural field conditions.

As shown in FIG. 15B the seed size of mCherry transgenic Brassica was significantly increased than that of WT, the 1000-seed weight was increased, grain yield per plant was increased. The result suggested that the increase of photosynthesis efficiency in mCherry transgenic Brassica leads to a significant increase of seed size and grain yield per plant.

### 2. Leaves of mCherry Transgenic Brassica Increase Light Energy Absorption

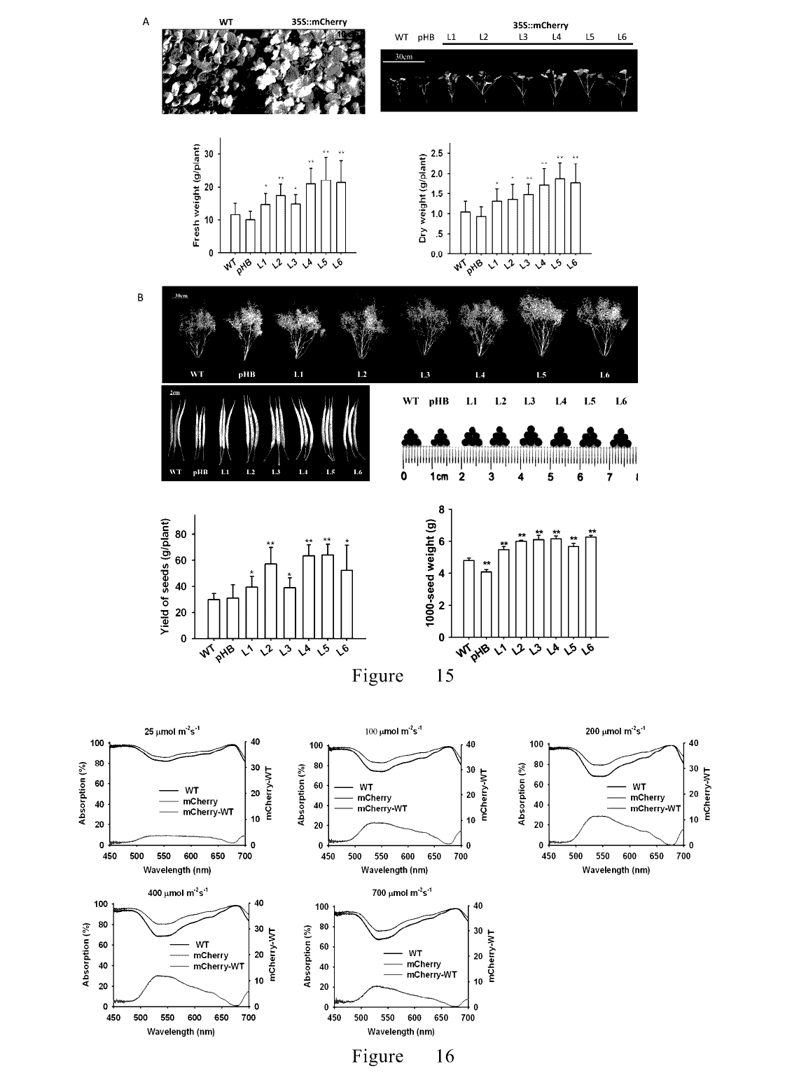

FIG. 16 showed the light absorbance spectra of mCherry transgenic Brassica leaves under different light intensities. The result suggested that the light energy absorbed by mCherry transgenic Brassica leaves was significantly increased.

### 3. Expression of mCherry in Brassica Enhanced the Cyclic and Linear Electron Flow Under White Light, Which Lead to the Changes in the Photochemical Efficiency of PSII and the Redox States of Plastoquinone Pool

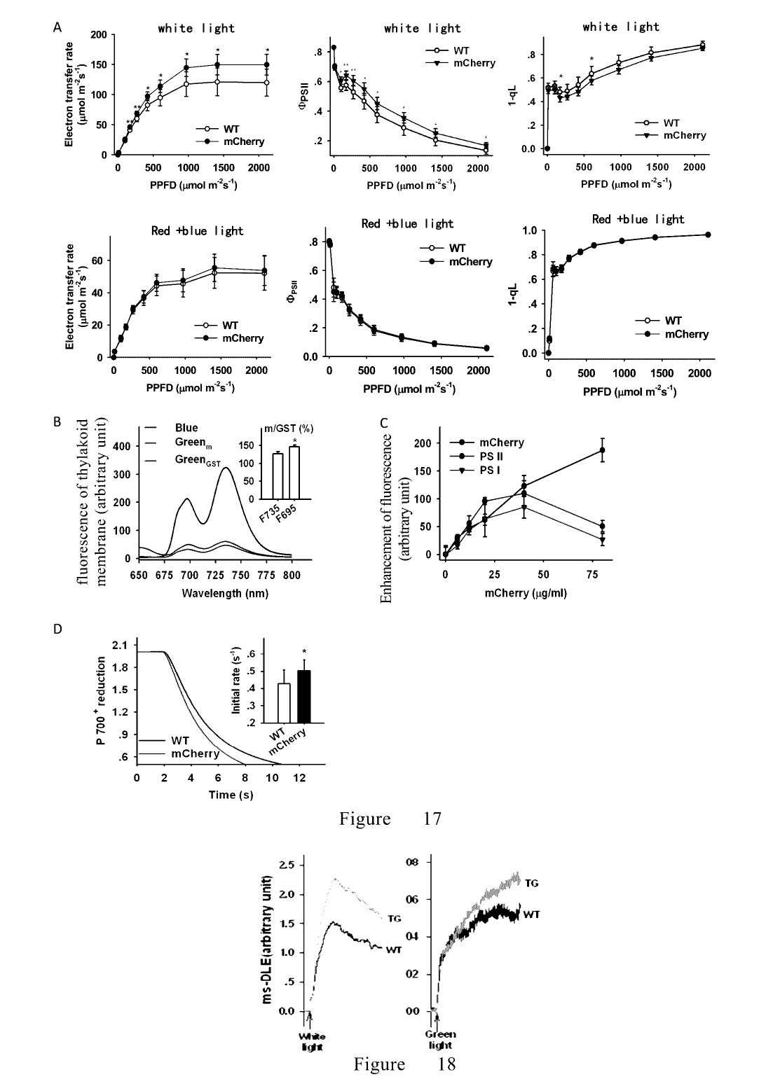

FIG. 17A showed the electron transfer rate, photochemical efficiency of PSII (Φ_{PSII}) and the excitation pressure of PSII (1−qL) of WT and mCherry transgenic Brassica plants grown under white light and red+blue light. Compared with WT, the slow phase of millisecond delay light evolution (ms-DLE) of mCherry transgenic Brassica was higher both under white light and under green light (FIG. 18). These results suggested that the enhancement of electron transfer lead to the increase of proton gradient across the thylakoid membrane (ΔpH) in the mCherry transgenic plants. Because ΔpH is used to the synthesis of ATP, the increase of ΔpH would benefit the ATP synthesis, and improve the photosynthesis.

FIG. 17B showed 77K Chl fluorescence of thylakoid membrane. The thylakoid membrane of WT was excited by blue light (wavelength: 435 nM). Or the thylakoid membrane of WT was excited by green light (wavelength: 540 nM) in the presence of 32 μg/ml of GST (Green_{GST}) or 32 μg/ml mCherry protein (Greenₘ).

FIG. 17C showed the 77k Chl a fluorescence spectra of the thylakoid membrane in the presence of different concentrations of mCherry protein. The black line (mCherry) represented the fluorescence intensity of GST-mCherry recombinant protein excited at 663 nm (the absorption peak of Chl a). In the absence of mCherry protein, the fluorescence intensity of thylakoid membrane excited by green light was much wreaker than that by blue light. When mCherry protein was added, no changes was observed in the fluorescence spectra of thylakoid membrane excited by green light, suggesting that mCherry protein did not change the light harvest complex and reaction center of photosynthesis, but increased the absorption of green light. The P700⁺ reduction of WT and mCherry transgenic Brassica. Inset: the initial rate of P700⁺ reduction. The initial rate of P700⁺ reduction represents the capacity of the cyclic electron transfer of PSI. The initial rate of P700⁺ reduction in leaves of mCherry transgenic Brassica (FIG. 17D) was much higher than that of WT, suggesting that the cyclic electron transfer rate around PSI was increased.

### 4. In mCherry Transgenic Brassica, Expression of Photosynthesis Related Proteins are Increased; Rubisco Activity is Increased; the State Transition Rate and Percentage are Increased Significantly

Western blot analysis of photosynthesis related proteins Atp B, D1, Psa D, Lhcb1, Lhca1 and cyt f in the leaves of WT and mCherry transgenic Brassica grown under white light or red+blue light. The results showed in FIG. 19A, suggested that expression of photosynthesis related proteins are significantly increased in mCherry transgenic Brassica grown under white light but no significant difference was observed between WT and mCherry transgenic Brassica when grown under red+blue light.

FIG. 19B showed Rubisco initial and total activity in Brassica. The result suggested that Rubisco activity and Rubisco activation state were increased in mCherry transgenic Brassica.

When measure the state transition of live leaf, the leaf was first illuminated with state 2 white light with PFD of 100 μmol m⁻²s⁻¹ for 15 minutes then state 1 infrared light (6 μmol m⁻²s⁻¹) was turned on for 15 minutes, after which the infrared light was turned off for another 15 minutes, t_{0.5} represents the half time for transition between state 1 (St1) and state 2 (St2). Result in FIG. 19C showed that t_{0.5} for state transition between St1 and St2 was significantly decreased in mCherry transgenic Brassica, suggesting that the rate of state transition is increased.

The capacity of state transition of thylakoid membrane was tested by the 77K fluorescence measurement. Thylakoid membrane was obtained from leaves of WT and mCherry transgenic Brassica, and the 77K fluorescence was measured. The spectra was normalized with the fluorescence peak at 685 nm. The result showed in FIG. 19D suggested that the percentage of state transition was increased when mCherry transgenic Brassica were grown under white light. This was consistent with the increase of Lhcbl content as shown by Western blot analysis. However, the result in FIG. 19E showed that no significant difference in state transition of thylakoid membrane was observed in Brassica grown under red+blue light, suggesting that the enhancement of state transition between the two photosystems under white light was related to the light absorbance of mCherry protein.

### 5. Chlorophyll Content in mCherry Transgenic Brassica Leaves was not Changed Significantly, but Contents of Accessory Photosynthetic Pigments (Carotenoids) were Increased Significantly

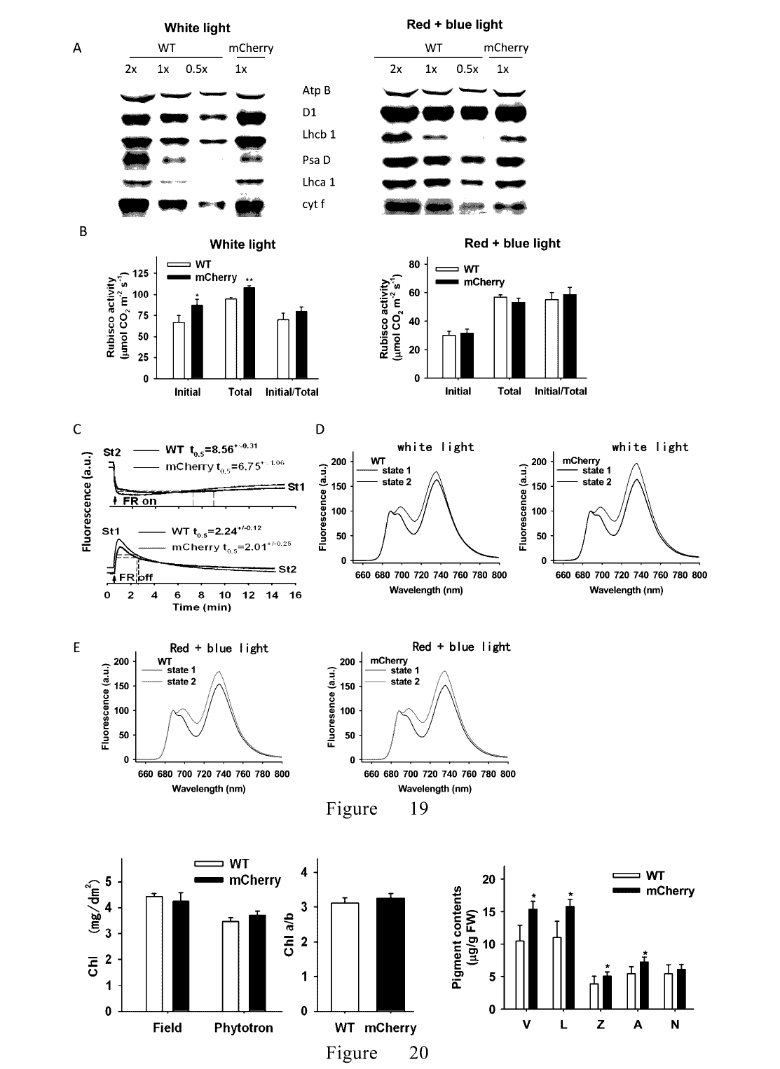

FIG. 20 showed the chlorophyll content (left) and the ratio of Chl a/b (middle) in mCherry transgenic Brassica and WT. The results suggested that expression of mCherry protein did not change the chlorophyll content and the ratio of Chl a/b in Brassica leaves.

The content of other photosynthetic pigments in the leaves of mCherry transgenic Brassica and WT was shown in FIG. 20 (right). In mCherry transgenic Brassica the major accessory photosynthetic pigments (carotenoids), including violaxanthin; lutein, zeaxanthin, antheraxanthin were increased.

The above results showed that the accessory photosynthetic pigments related to the energy dissipation under strong light was increased, indicating the increased capacity of light energy dissipation in transgenic plant.

### 6. Net Photosynthesis Rate of mCherry Transgenic Brassica Increased Together with Stomatal Conductance Increased

FIG. 21A showed the intercellular CO₂ concentration (Ci) and FIG. 21B showed the stomatal conductance (Gs) of mCherry transgenic Brassica grown in the phytotron under white light (light intensity: 250 μmol m⁻²s⁻¹). FIG. 21C showed the Ci and Gs of field grown mCherry transgenic Brassica at 11-week-old. The results indicated that the increased net photosynthesis rate together with increased Gs did not lead to the decrease of intercellular CO₂ concentration in mCherry transgenic Brassica leaves.

FIG. 21D is the net photosynthesis rate versus Ci responsive curve under saturation light. The result of CO₂ responsive curve indicated that the increase in Gs contributes a low proportion to the increase of photosynthesis rate, and the increase of photosynthesis rate is mainly due to the increases of electron transfer capacity and Rubisco activity.

### 7. Expression of mCherry Protein Improved the Photochemical Efficiency of PSII, Contents or Percentage of Redox Metabolites, such as NADH, NADPH, GSH and Ascorbate.

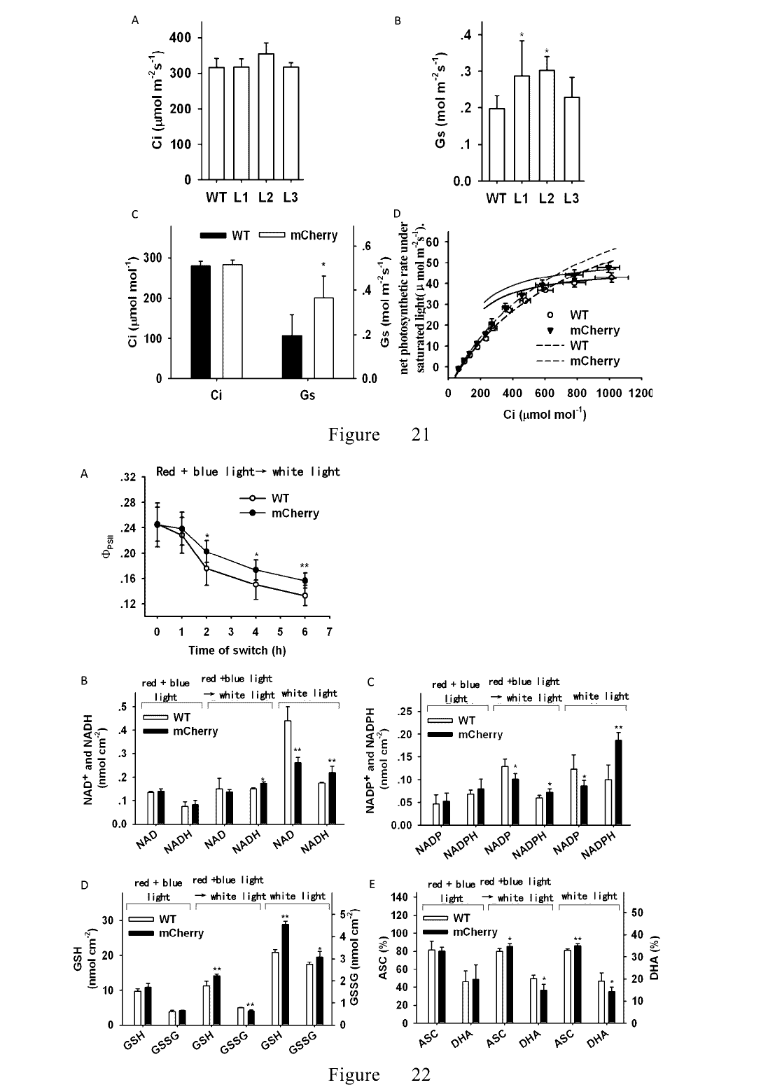

Expression of mCherry in the leaves of Brassica increased the percentage of reduced form of redox metabolites. WT and mCherry transgenic Brassica plants grown under red+blue light (red light: 60 μmol m⁻²s⁻¹, blue light: 20 μmol m⁻²s⁻¹ ) were transferred to white light (light intensity: 80 μmol m⁻²s⁻¹). The changes in Φ_{PSII} (FIG. 22A). the contents of NAD⁺ and NADH (FIG. 22B), the contents of NADP⁺ and NADPH (FIG. 22C), the contents of GSH and GSSG (FIG. 22D), the content of ascorbate (ASC) and dehydroascorbate (DHA) (FIG. 22E) were measured. The above results indicated that expression of mCherry protein increased Φ_{PSH}, the content of reduced metabolites or the ratio of reduced to oxidized metabolites, such as NADH, NADPH, ascorbate and reduced glutathione (GSH), which lead to the improvement of redox status in mCherry transgenic Brassica.

### 8. Light Induced O₂ Evolution in the Etiolated Hypocotyl of mCherry Transgenic Brassica when the Respiration was Blocked

The O₂ evolution of the etiolated hypocotyl of mCherry transgenic Brassica was measured under light. The changes in soluble O₂ in dark represents the respiration (O₂ uptake) rate of the hypocotyls. And the changes in soluble O₂ in light reflected the mixed effects of O₂ evolution and respiration of hypocotyls. The differential value of the two above rate represents the O₂ evolution of hypocotyls. Because chloroplasts were not developed in the etiolated hypocotyls, the differential value may indicate the O₂ evolution catalyzed by mCherry protein in etiolated seedling hypocotyl. HgCl₂ is used widely as a terminal oxide. Both the mitochondrion respiration and the O₂ evolution from chloroplasts are almost inhibited when high concentration of HgCl₂ is presented. However, the activity of mCherry cannot be inhibited by HgCl₂ because mCherry protein does not possess a sulfhydryl group (no cysteine residue), the net O₂ evolution rate can be measured directly in the presence of HgCl₂ which can exclude the disturbance of the plastid respiration in hypocotyls. −HgCl₂: differential value of O₂ uptake in dark and under light without HgCl₂ in the assay; +HgCl₂: net O₂ evolution when the respiration was inhibited with HgCl₂. FIG. 23 showed that the O₂ uptake rate of mCherry transgenic Brassica is significant lower than that of WT under light, moreover, net O₂ evolution was observed significantly when the O₂ uptake was inhibited by HgCl₂, suggesting that net O₂ evolution can be measured when the O₂ uptake was inhibited in the mCherry transgenic Brassica as compared with WT. The results indicating that ectopic expression of mCherry in transgenic Brassica could catalyzed O₂ evolution by using some unknown derivatives of methyl quinone in vivo, for example, plastoquinone, etc. It implies that mCherry could form a photoreaction system in vivo which is similar to the in vitro reaction, and could utilize the light energy continually to produce reduced form of quinone.

### 9. Expression of mCherry Improved the Growth of Rice at Young Seedling Stage

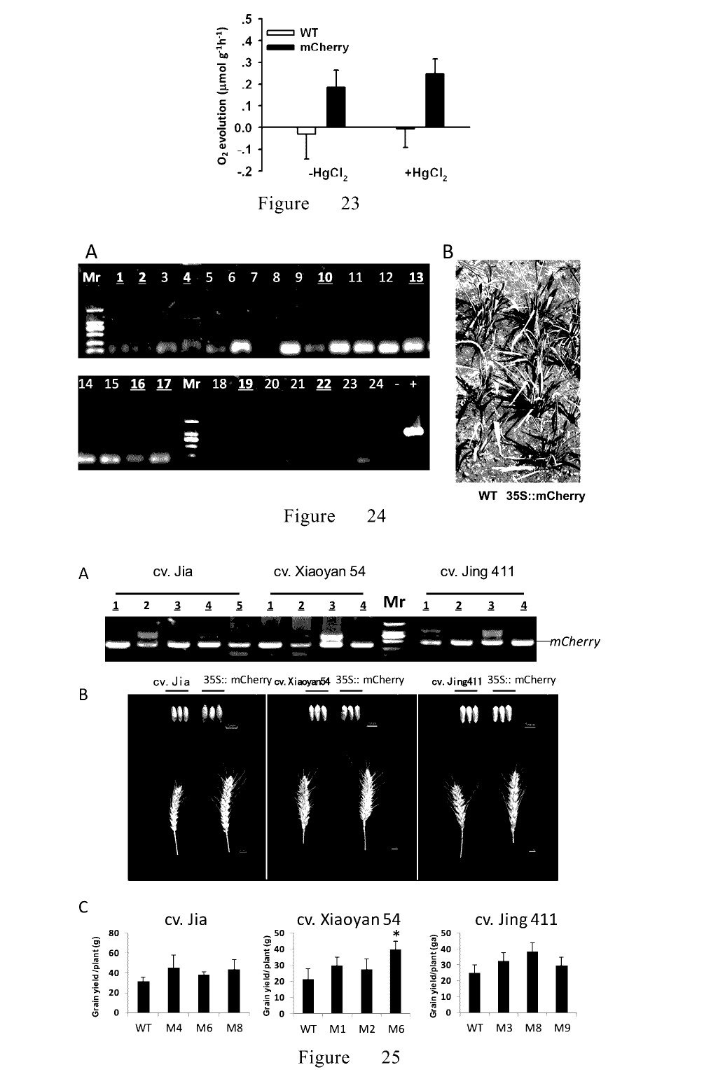

Compared the growth of rice seedling between WT (9311) and T1 generation of mCherry transgenic rice (FIG. 24). The results showed that the expression of mCherry improve the growth of rice at young seedling stage.

### 10. mCherry Transgenic Wheat

The phenotype of mCherry transgenic wheat was further analyzed. Ectopic expression of mCherry in three wheat cultivars, Jia, Xiaoyan 54 and Jin 411 significantly increased the size of seed and panicle in T2 generation as shown in FIG. 25.

### Example 4

### Functional Study on LEAT Protein (BFP) Transgenic Plants

### 1. Expression of BFP in Transgenic Brassica Stimulates the Growth and Biomass Accumulation of Brassica Grown in the Natural Field Conditions

BFP transgenic Brassica plants were identified when the transgenic Brassica were obtained. The result of identification of BFP transgenic Brassica genome was shown in FIG. 26A. Five lines (B1-B5) contain BFP gene integration and therefore are positive transgenic lines. Result of RT-PCR analysis of BFP expression in transgenic Brassica line B1-B5 were shown in FIG. 26B, which suggests that BFP is expressed in transgenic lines B1-B5. T2generation of BFP transgenic Brassica plants were used for all the experiments in this invention.

The 7-week-old BFP transgenic Brassica and WT grown in the natural field conditions and their fresh weight and dry weight were shown in FIG. 26C. Comparison of the size of whole plant, silique and seed as well as grain yield per plant at the harvest time was shown in FIG. 26D. Thus, expression of BFP in Brassica stimulates the growth and biomass accumulation of Brassica grown in the natural field conditions. When compare the T2 seeds of BFP transgenic Brassica with WT, it was found that the seed size of BFP transgenic Brassica was significantly increased and the grain yield per plant was increased (FIG. 26D). This result suggests that BFP transgenic Brassica increases photosynthesis rate and significantly increases seed size and grain yield per plant.

### 2. Expression of BFP in Transgenic Brassica Stimulates the Plant Growth and Resistance to UV

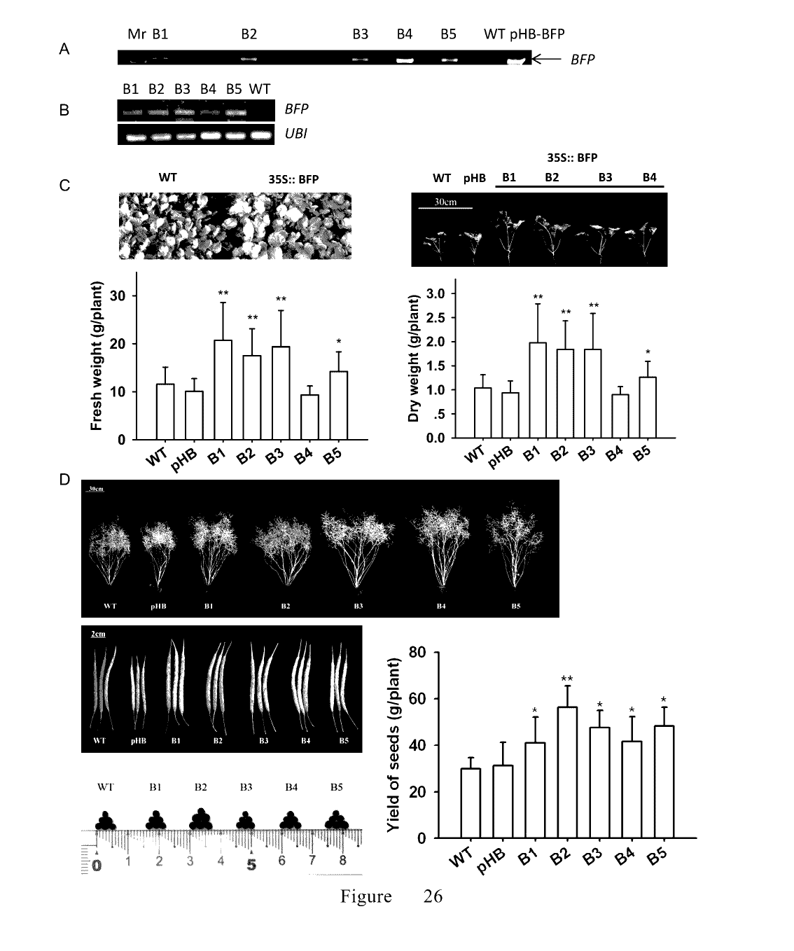

WT, empty vector control (pHB) and BFP transgenic Brassica (B1-B5) were germinated and grown under white light in the phytotron (light intensity: 250 μmol m^{31 2}s⁻¹, UV-B radiation intensity: 0.013 mW cm⁻²) 1 week. 1-week-old young seedlings were switched to white light or white light+UV (UV-B radiation intensity: 0.075 mW cm⁻²) conditions and grew for another three weeks. Photos were taken three weeks after switch. The results were shown in FIGS. 27A and 27B. Therefore, BFP expression in transgenic Brassica stimulates the plant growth and increases the resistance to UV radiation.

### 3. The Net Photosynthesis Rate of BFP Transgenic Brassica Increased when Grown in the Natural Field Conditions. The Net Photosynthesis Rate was Significantly Increased Under High Light Conditions Even when Measured with Red+Blue Light Source

The net photosynthesis rate was measured in situ, and the measuring time was set between 10:00 am-12:00 pm. The measuring light source was sunlight. The net photosynthesis rates of WT and BFP transgenic Brassica under different light intensities were shown in FIG. 28. The net photosynthesis rate of BFP transgenic Brassica was significantly higher than WT under high light conditions, whereas under low light conditions, there was no significant difference.

### 4. The Generation of Photochemical Efficiency of PSII and the Redox Status of Plastoquinone in the BFP Transgenic Brassica was Affected

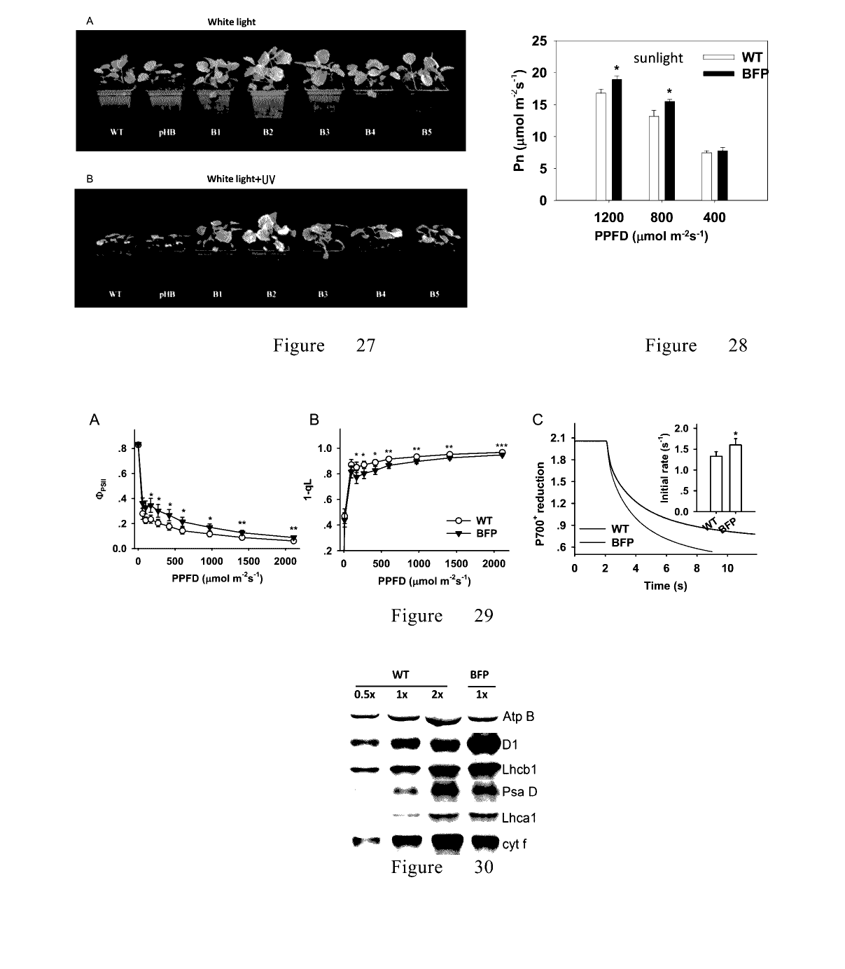

The photochemical efficiency of PSII of WT and BFP transgenic Brassica was compared and the result was shown in FIG. 29A. The excitation pressure of PSII (1−qL) of WT and BFP transgenic Brassica was measured and the result was shown in FIG. 29B. 1−qL represents the redox status of the plastoquinone pool. The reduction kinetics of P700⁺ of WT and BFP transgenic Brassica was shown in FIG. 29C. The reduction kinetics of P700⁺ reflects the cyclic electron transport capacity. Therefore, the photochemical efficiency of PSII and the PSI cyclic electron transport was increased, the redox status of plastoquinone pool was influenced in the BFP transgenic Brassica.

### 5. Expression of Photosynthesis Related Proteins are Increased in the Leaves of BFP Transgenic Brassica

Expression of photosynthesis related proteins Atp B, D1, Psa D, Lhcb1, Lhca1 and cyt f in the leaves of WT and BFP transgenic Brassica was analyzed by Western blot. The results were shown in FIG. 30. The results suggest expression of photosynthesis related proteins are significantly in creased in BFP transgenic Brassica.

### 6. Expression of BFP Increase the Chl a/b Ratio and the Carotenoid Contents in Leaves

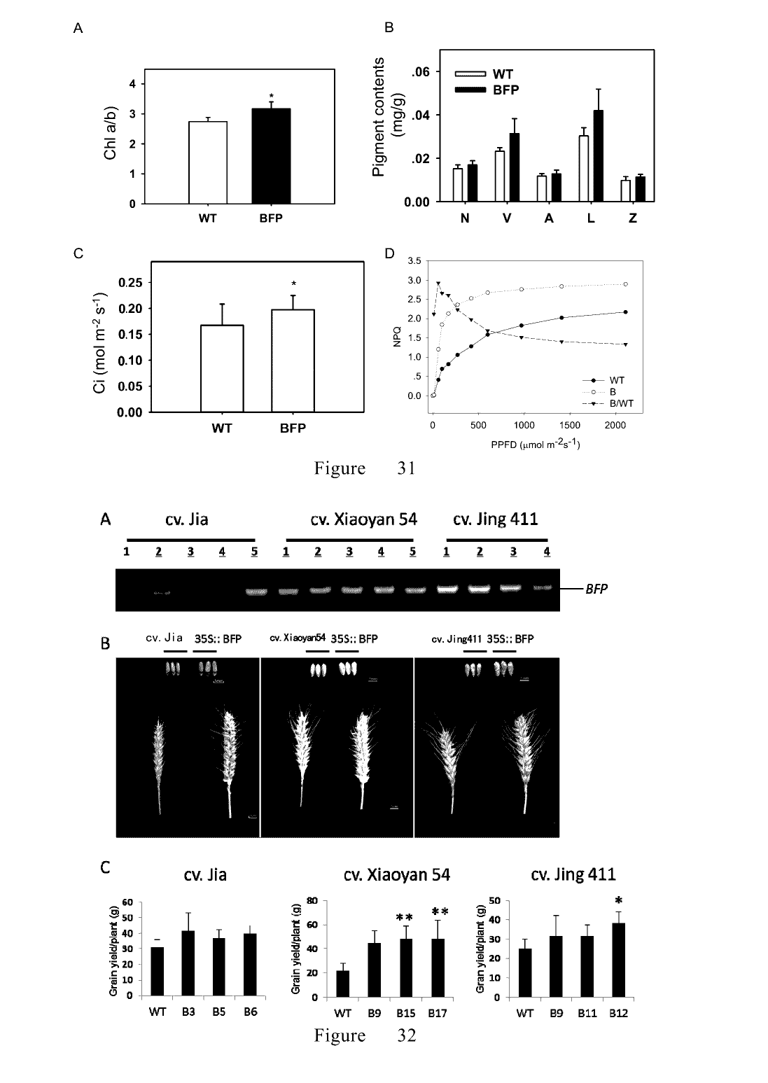

The Chl a/b ratio was shown in FIG. 31A. Contents of different carotenoids were shown in FIG. 31B. The results suggested BFP expression increased the Chl a/b ratio and the carotenoid contents. Carotenoids are accessory photosynthetic pigments. Increase in caroteinoid contents may increase the dissipation of light energy under high light conditions and increase the light protection ability of plants.

### 7. Measurement of Stomatal Conductance

Stomatal conductance of WT and BFP transgenic Brassica was measured in situ in the field by using LI-6400. The PPFD of sunlight during measurement was 1200 μmol m⁻²s⁻¹. The result was shown in FIG. 31C. The stomatal conductance of BFP transgenic Brassica was increased significantly, which makes CO₂, the substrate of photosynthetic carbon assimilation, more easily to be diffused in leaves and therefore increases the efficiency of photosynthetic carbon assimilation.

### 8. Changes in the Light Protection Ability

The light protection abilities of BFP transgenic Brassica and WT were measured. Non-photochemical quenching (NPQ) of transgenic Brassica leaves was measured by using conventional method. The result showed the non-photochemical quenching of BFP transgenic Brassica was significantly increased (FIG. 31D), suggesting that the light protection ability of the transgenic Brassica was enhanced.

### 9. BFP Transgenic Wheat

Further experiments showed that expression of BFP in wheat had similar phenotype. BFP was expressed in three cultivars of wheat, i.e. Jia, Xiaoyan 54 and Jing 411. T2generation of the BFP transgenic wheat showed increased seed size and panicle size (FIG. 32).

### Example 5

### LEAT Protein mGFP5 Transgenic Crops

### 1. Phenotype of Seeds and Panicles of mGFP5 Transgenic Wheat

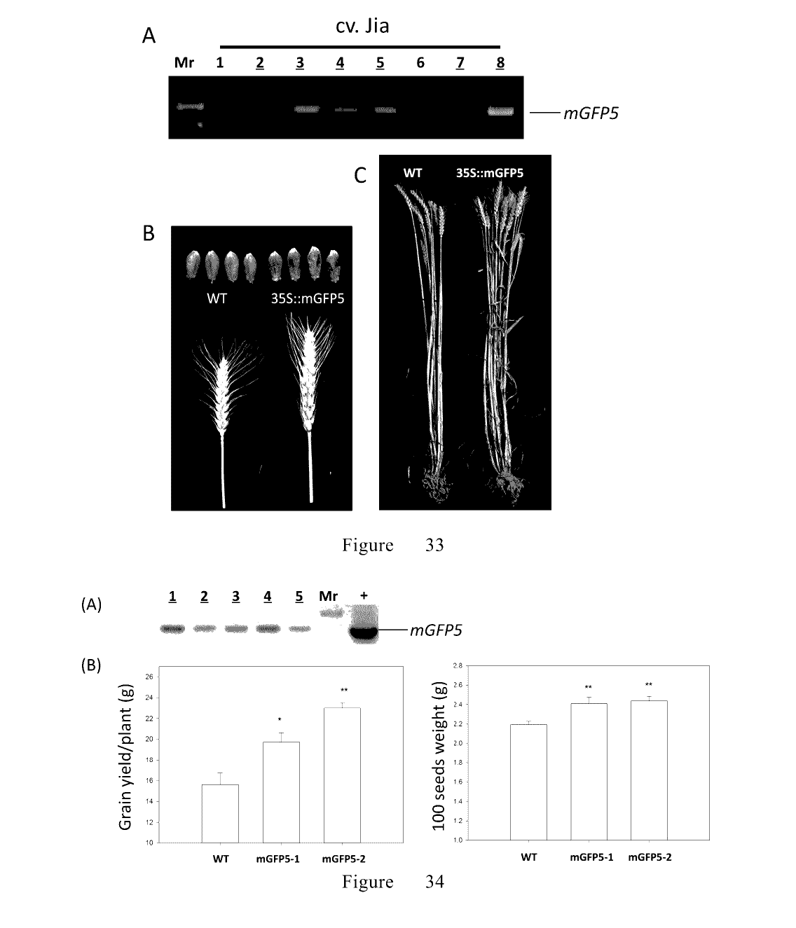

mGFP5 was expressed in wheat (cultivar Jia). The seed size and panicle size of mGFP5 transgenic wheat was significantly increased (FIG. 33B). The tiller number and panicle number are also increased (FIG. 33C).

### 2. mGFP5 Transgenic Rice Increased the Tiller Number Per Plant, Panicle Number, Grain Yield Per Plant and 100-Seed Weight

mGFP5 was expressed in rice (cultivar Zhonghua 11). The average tiller number is 8.1 and panicle number is 7.4 (n=10) which were significantly increased than those of WT cultivar Zhonghua 11 (tiller number=5.8, panicle number=5.8). The 100-seed weight and grain yield per plant were also significantly increased (FIG. 34B).

### 3. Biomass, Boll Number and Boll Weight Per Plant were Increased in mGFP5 Transgenic Cotton

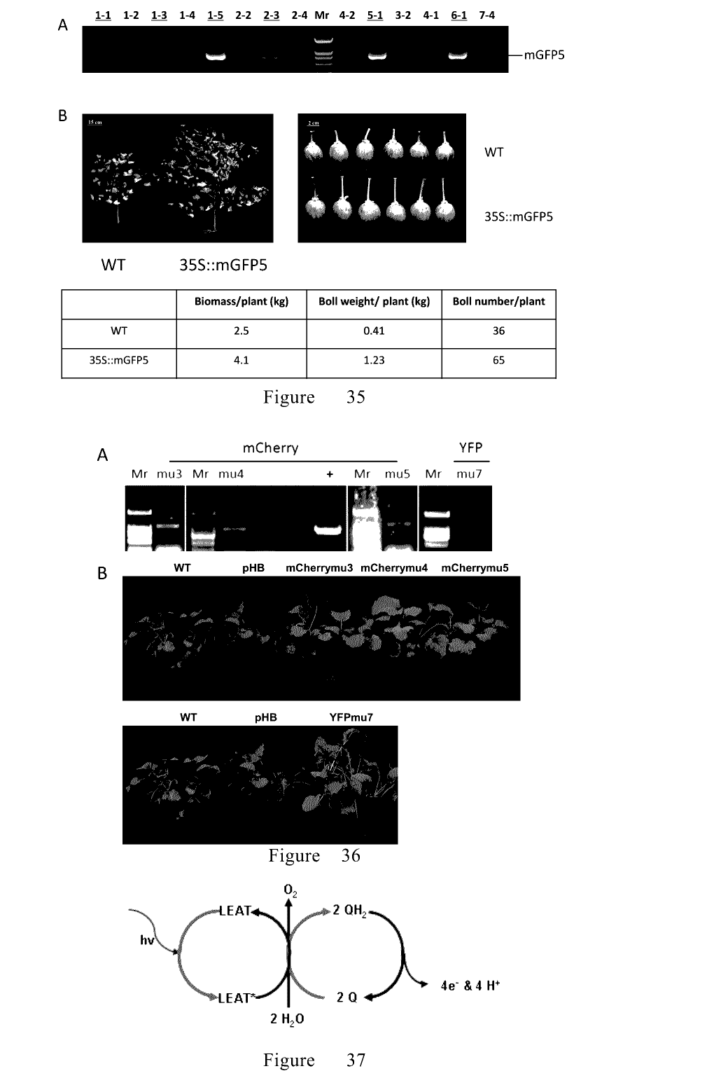

Ectopic expression of mGFP5 in cotton significantly increased the plant height in T3generation. The biomass per plant was 2.5 kg, the weight of boll was 1.2 kg and the number of boll was 65 per plant, which were significantly higher than those of WT (FIG. 35B).

### Example 6

### Functional Study on Low Fluorescent LEAT Mutant Transgenic Brassica

### 1. Expression of Non-Fluorescent or Low Fluorescent YFP and mCherry Mutant Genes in Brassica Stimulate the Growth of Brassica at Young Seedling Stage

The comparison of the growth of T1 generation transgenic Brassica with non-fluorescent or low fluorescence mCherry mutants (mCherrymu3, mCherrymu4 and mCherrymu5) and YFP mutant (YFPmu7) was shown in FIG. 36B. YFP and mCherry mutant transgenic Brassica grow better than WT at vegetative stage.

The above examples 1-6 indicate: LEAT proteins that are excited by light can catalyze H₂O hydrolysis, and transfer the electrons and protons generated from the hydrolysis to the quinones, especially plastoquinones that exist in plant cells and therefore accomplish the oxygen evolution and quinone reduction. This catalysis character exists generally in LEAT proteins, but the quinones with proper structures are required for the catalytic reaction. The electrons and protones stored in the reduced quinones are further transferred through enzymetic oxydation-reduction reactions in the cytosol, which may regulate the redox status in associate with ascorbic acid or NAD(P) and finally enhance the photosynthetic apparatus gene expression and photosynthesis efficiency.

It should be noted that, in the present invention, all of the documents referred to in this application by reference, as if each reference were individually incorporated by reference that. It should also be understood that the above specific embodiments of the present invention and by the use of technical principles, after reading the contents of the present invention described above, the person skilled in the art can make various modifications of the present invention or modifications without departing from the invention. The spirit and scope of these equivalent forms also fall within the scope of the present invention.

1. A method for improving plant traits, comprising the steps of:
1) transforming one or more polynucleotides encoding light energy absorption and transduction protein into plants;
2) selecting the plants with improved traits compared with a control plant from the transformed plants;
said light energy absorption and transduction protein is a protein which can utilize light energy to catalyze water hydrolysis and 2,3,5-trimethyl-1,4-benzoquinone reduction.

2. The method of claim 1, wherein the polynucloetide encoding light energy absorption and transduction protein is selected from the group consisting of:
(a) a polynucleotide encoding a fluorescent protein or the mutant proteins thereof with one or more amino acid side mutations which are changed in fluorescence intensity and fluorescence emission spectra but can still utilize light energy to catalyze water hydrolysis and 2,3,5-trimethyl-1,4-benzoquinone reduction; or
(b) a polynucleotide encoding a non-fluorescent chromoprotein or the mutants thereof with one or more amino acid site mutations which can still utilize light energy to catalyze water hydrolysis and 2,3,5-trimethyl-1,4-benzoquinone reduction.

3. The method of claim 2, wherein the polynucloetide encoding a fluorescent protein or the mutant proteins thereof with one or more amino acid site mutations which are changed in fluorescence intensity and fluorescence emission spectra but can still utilize light energy to catalyze water hydrolysis and 2,3,5-trimethyl-1,4-benzoquinone reduction is selected from the group consisting of:
(a) a polynucleotide encoding a protein with the animo acid sequence as set forth in SEQ ID NO: 4;
(b) a polynucleotide encoding a protein with the animo acid sequence as set forth in SEQ ID NO: 10;
(c) a polynucleotide encoding a protein with the animo acid sequence as set forth in SEQ ID NO: 36;
(d) a polynucleotide encoding a protein with the animo acid sequence as set forth in SEQ ID NO: 6;
(e) a polynucleotide encoding a protein with the animo acid sequence as set forth in SEQ ID NO: 28;
(f) polynucleotide encoding a protein with the animo acid sequence as set forth in SEQ ID NO: 40;
(g) a polynucleotide encoding a protein with the animo acid sequence as set forth in SEQ ID NO; 44;
(h) a polynucleotide encoding a protein with the animo acid sequence as set forth in SEQ ID NO: 52;
(i) a polynucleotide encoding a protein with the animo acid sequence as set forth in SEQ ID NO: 8;
(j) a polynucleotide encoding a protein with the animo acid sequence as set forth in SEQ ID NO: 12;
(k) a polynucleotide encoding a protein with the animo acid sequence as set forth in SEQ ID NO: 14:
(l) a polynucleotide encoding a protein with the animo acid sequence as set forth in SEQ ID NO: 16;
(m) a polynucleotide encoding a protein with the animo acid sequence as set forth in SEQ ID NO: 18;
(n) a polynucleotide encoding a protein with the animo acid sequence as set forth in SEQ ID NO: 20;
(o) a polynucleotide encoding a protein with the animo acid sequence as set forth in SEQ ID NO: 50;
(p) a polynucleotide encoding a protein with the animo acid sequence as set forth in SEQ ID NO: 2;
(q) a polynucleotide encoding a protein with the animo acid sequence as set forth in SEQ ID NO: 22;
(r) a polynucleotide encoding a protein with the animo acid sequence as set forth in SEQ ID NO: 24;
(s) a polynucleotide encoding a protein with the animo acid sequence as set forth in SEQ ID NO: 26;
(t) a polynucleotide encoding a protein with the animo acid sequence as set forth in SEQ ID NO: 30;
(u) a polynucleotide encoding a protein with the animo acid sequence as set forth in SEQ ID NO: 34;
(v) a polynucleotide encoding a protein with the animo acid sequence as set forth in SEQ ID NO: 42;
(w) a polynucleotide encoding a protein with the animo acid sequence as set forth in SEQ ID NO: 46;
(x) a polynucleotide encoding a protein with the animo acid sequence as set forth in SEQ ID NO: 32;
(y) a polynucleotide encoding a protein with the animo acid sequence as set forth in SEQ ID NO: 48;
(z) a polynucleotide encoding a protein with the animo acid sequence as set forth in SEQ ID NO: 62;
(aa) a polynucleotide encoding a protein with the animo acid sequence as set forth in SEQ ID NO: 64;
(ab) a polynucleotide encoding a protein formed by one or more amino acid residue substitution, deletion or addition in any of the amino acid sequences in (a) to (aa) and having the ability to utilize light energy to catalyze water hydrolysis and 2,3,5-trimethyl-1,4-benzoquinone reduction;
(ac) a polynucleotide encoding a protein which has more than 70% sequence identity to the protein with any of the amino sequences in (a) to (aa) and having the ability to utilize light energy to catalyze water hydrolysis and 2,3,5-trimethyl-1,4-benzoquinone reduction; or
(ad) a polynucleotide complementary to any of the polynucleotide in (a) to (ac).

4. The method of claim 3, wherein the polynucleotide encoding a fluorescent protein or the mutant proteins thereof with one or more amino acid site mutations which are changed in fluorescence intensity and fluorescence emission spectra but can still utilize light energy to catalyze water hydrolysis and 2,3,5-trimethyl-1,4-benzoquinone reduction is selected from the groups consisting of:
(a) a polynucleotide encoding the nucleotide sequence as set forth in SEQ ID NO: 3;
(b) a polynucleotide encoding the nucleotide sequence as set forth in SEQ ID NO: 9;
(c) a polynucleotide encoding the nucleotide sequence as set forth in SEQ ID NO: 35;
(d) a polynucleotide encoding the nucleotide sequence as set forth in SEQ ID NO: 5;
(e) a polynucleotide encoding the nucleotide sequence as set forth in SEQ ID NO: 27;
(f) a polynucleotide encoding the nucleotide sequence as set forth in SEQ ID NO: 39;
(g) a polynucleotide encoding the nucleotide sequence as set forth in SEQ ID NO: 43;
(h) a polynucleotide encoding the nucleotide sequence as set forth in SEQ ID NO: 51;
(i) a polynucleotide encoding the nucleotide sequence as set forth in SEQ ID NO: 7;
(j) a polynucleotide encoding the nucleotide sequence as set forth in SEQ ID NO: 11;
(k) a polynucleotide encoding the nucleotide sequence as set forth in SEQ ID NO: 13;
(l) a polynucleotide encoding the nucleotide sequence as set forth in SEQ ID NO: 15;
(m) a polynucleotide encoding the nucleotide sequence as set forth in SEQ ID NO: 17;
(n) a polynucleotide encoding the nucleotide sequence as set forth in SEQ ID NO: 19;
(o) a polynucleotide encoding the nucleotide sequence as set forth in SEQ ID NO: 49;
(p) a polynucleotide encoding the nucleotide sequence as set forth in SEQ ID NO: 1;
(q) a polynucleotide encoding the nucleotide sequence as set forth in SEQ ID NO: 21;
(r) a polynucleotide encoding the nucleotide sequence as set forth in SEQ ID NO: 23;
(s) a polynucleotide encoding the nucleotide sequence as set forth in SEQ ID NO: 25;
(t) a polynucleotide encoding the nucleotide sequence e set forth in SEQ ID NO: 29;
(u) a polynucleotide encoding the nucleotide sequence as set forth in SEQ ID NO: 33;
(v) a polynucleotide encoding the nucleotide sequence as set forth in SEQ ID NO: 41;
(w) a polynucleotide encoding the nucleotide sequence as set forth in SEQ ID NO: 45;
(x) a polynucleotide encoding the nucleotide sequence as set forth in SEQ ID NO: 31;
(y) a polynucleotide encoding the nucleotide sequence as set forth in SEQ ID NO: 47;
(z) a polynucleotide encoding the nucleotide sequence as set forth in SEQ ID NO: 61;
(aa) a polynucleotide encoding the nucleotide sequence as set forth in SEQ ID NO: 63; or
(ab) a polynucleotide complementary to any of the polynucleotide in (a) to (aa).

5. The method of claim 2, wherein the polynucleotide encoding a non-fluorescent chromoprotein or the mutant proteins thereof with one or more amino acid site mutations which can still utilize light energy to catalyze water hydrolysis and 2,3,5-trimethyl-1,4-benzoquinone reduction is selected from the group consisting of:
(a) a protein with the amino acid sequence as set forth in SEQ ID NO: 38;
(b) a protein formed by one or more amino acid residue substitution, deletion or addition in the amino acid sequence as set forth in (a) and having the ability to utilize light energy to catalyze water hydrolysis and 2,3,5-trimethyl-1,4-benzoquinone reduction;
(c) a protein having more than 70% sequence identity to the protein with any amino sequence as set forth in (a) and having the ability to utilize light energy to catalyze water hydrolysis and 2,3,5-trimethyl-1,4-benzoquinone reduction; or
(d) a polynucleotide complementary to any of the polynucleotide in (a) to (c).

6. The method of claim 5, wherein the polynucleotide encoding a non-fluorescent chromoprotein or the mutant proteins there of with one or more amino acid site mutations which can still utilize light energy to catalyze water hydrolysis and 2,3,5-trimethyl-1,4-benzoquinone reduction is selected from the group consisting of:
(a) a polynucleotide with the nucleotide sequence as set forth in SEQ ID NO: 37; or
(b) a polynucleotide complementary to the polynucleotide of (a).

7. The method of claim 2, wherein the polynucleotide encoding light energy absorption and transduction protein is further selected from the group consisting of: a polynucleotide encoding the protein selected from the following Genbank accession numbers; AF168421, AY646070, AY646072, AY646071, AF168420, AY182022, AY182023, DQ206381, DQ206392, DQ206382, AY181556, AY679113, EU498721, AY182017, AY646069, AY646066, AY151052, EU498722, AY647156, AY508123, AY508124, AY508125, AF435432, AY646067, AY485334, AY485335, AY646068, AF545827, AF545830, AY037776, AB180726, DQ206383, DQ206395, DQ206396, DQ206385, EU498723, AB193294, AY182020, AY182021, AB107915, DQ206389, P42212, AY268073, AY181553, AY181554, AY181555, DQ206393, AY155344, AY037766, AY679112, AF401282, EU498724, AY268076, AY268074, AY268075, AY268071, AY268072, DQ206391, AY015995, AY182014, AF372525, EU498725, DQ206390, AF168422, AY646073, AY296063, AF272711, AB128820, DQ206379, DQ206380, AF168419, AY059642, EF186664, AF420591, DQ206387, AY182019, AY765217, AB085641, AY181552, DQ206386, AY182013, AY646064, AY485333, AF168423, AY646077, AY646076, AY646075, EF587182, AF363775, AF383155, DQ206394, DQ206373, AF38315, AF363776, AY461714, AB209967, DQ206377, DQ206378, or the mutant proteins thereof with one or more amino acid site mutations which are changed in fluorescence intensity and fluorescence emission spectra but can still utilize light energy to catalyze water hydrolysis and 2,3,5-trimethyl-1,4-benzoquinone reduction.

8. The method of claim 1, wherein the method for transforming the polynucleotide into plants comprises: transforming the expression cassette containing the nucleotide encoding the sight, energy absorption and transduction protein into planes, thereby expressing the nucleotide in the plants.

9. The method of claim 1, wherein the improved plant trait is one or more traits selected from the group consisting of:
increasing the biomass of a plant;
increasing the yield of a plant;
promoting the growth of a plant
increasing the size of seed or panicle of a plant;
increasing the number of seeds, tillers or panicles of a plant;
increasing seed size;
increasing the seed weight;
increasing the total content of protein of a plant;
increasing light utilization efficiency of a plant;
increasing photochemical efficiency of PSI or PSII of a plant;
increasing photosynthesis electron transfer efficiency of a plant;
increasing CO₂ assimilation ability of a plant;
increasing net photosynthetic rate of a plant;
increasing the light protection ability of a plant;
increasing the content of accessory photosynthetic pigment in a plant;
increasing the photosynthetic O₂ evolution rate of a plant.

10. The method of claim 1, wherein the plants are gymnosperms, monocotyledonous or dicotyledonous plants.

11. A method for improving plant traits, comprising transforming one or more polynucleotides encoding light energy absorption and transduction protein into plants.

12. A separated light energy absorption and transduction protein, comprising:
a protein with the animo acid sequence as set forth in SEQ ID NO: 22;
a protein with the animo acid sequence as set forth in SEQ ID NO: 24;
a protein with the animo acid sequence as set forth in SEQ ID NO: 26;
a protein with the animo acid sequence as set forth in SEQ ID NO: 12;
a protein with the animo acid sequence as set forth in SEQ ID NO: 14;
a protein with the animo acid sequence as set forth in SEQ ID NO: 16;
a protein with the animo acid sequence as set forth in SEQ ID NO: 14;
a protein with the animo acid sequence as set forth in SEQ ID NO: 16
a protein with the animo acid sequence as set forth in SEQ ID NO: 18;
a protein with the animo acid sequence as set forth in SEQ ID NO: 20;
a protein with the animo acid sequence as set forth in SEQ ID NO: 62; or
a protein with the animo acid sequence as set forth in SEQ ID NO: 64.

13. A separated polynucleotide encoding any of the light energy absorption and transduction proteins of claim 12.

14. A recombinant expression vector, wherein the vector comprises the polynucleotide of claim 13.

15. A genetic engineered cell wherein the cell comprises the recombinant expression vector of claim 14, or the genome of the cell is integrated with the polynucleotide encoding any of the light energy absorption and transduction proteins selected from:
a protein with the amino acid sequence as set forth in SEQ ID NO: 22;
a protein with the amino acid sequence as set forth in SEQ ID NO: 24;
a protein with the amino acid sequence as set forth in SEQ ID NO: 26;
a protein with the amino acid sequence as set forth in SEQ ID NO: 12;
a protein with the amino acid sequence as set forth in SEQ ID NO: 14;
a protein with the amino acid sequence as set forth in SEQ ID NO: 16;
a protein with the amino acid sequence as set forth in SEQ ID NO: 18;
a protein with the amino acid sequence as set forth in SEQ ID NO: 20;
a protein with the amino acid sequence as set forth in SEQ ID NO: 62; or
a protein with the amino acid sequence as set forth in SEQ ID NO: 64.

16. A transgenic plant and the cell tissue or progeny thereof prepared by the method of claims 1, wherein the transgenic plant or the progeny thereof possess improved traits compared with a control plant.

17. A method for generating plant seeds with improved traits, comprising culture the transgenic plant prepared ay any of the method of claims 1.


SEQUENCE LISTING (64 sequences)

SEQ ID NO: 1 - DNA, 711 bp, Discosoma sp.
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 2 - protein, 236 aa, Discosoma sp.
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 3 - DNA, 720 bp, Aequorea
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagccacggcgtccagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacagccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 239 aa, Aequorea
MVSKGEELFTGVVPILVELDGDVNGHKFSVSGEGEGDATYGKLTLKFICTTGKLPVPWPTLVTTLSHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKANFKIRHNIEDGSVQLADHYQQNTPIGDGPVLLPDSHYLSTQSALSKDPNEKRDHMVLLEFVTAAGITLGMDELYK

SEQ ID NO: 5 - DNA, 720 bp, Aequorea
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 6 - protein, 239 aa, Aequorea
MVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 7 - DNA, 720 bp, Aequorea Victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 8 - protein, 239 aa, Aequorea Victoria
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 9 - DNA, 720 bp, Aequorea Victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - protein, 239 aa, Aequorea Victoria
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 11 - DNA, 720 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagctgtaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcgcacagtccgccctgagcaaagaccccaacgagaagcgcgatcacatggtcctgctggagttcgtgaccgccgccgggatcactctcggcatggacgagctgtacaagtaa

SEQ ID NO: 12 - protein, 239 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSCNVYIMADKQKNGIKVNFKIRHNIEDGSVQLADHYQQNTPIGDGPVLLPDNHYLSAQSALSKDPNEKRDHMVLLEFVTAAGITLGMDELYK

SEQ ID NO: 13 - DNA, 720 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagctgtaacgtctatatcatggccgacaagcagaagaacggcatcaaggtgaacaacaagatgcgccacaacatcgaggacggcagcgtgcagctcgccgaccactaccagcagaacacccccatcggcgacggccccgtgctgctgcccgacaaccactacctgagcgcacagtccgccctgagcaaagaccccaacgagaagcgcgatcacatggtcctgctggagttcgtgaccgccgccgggatcactctcggcatggacgagctgtacaagtaa

SEQ ID NO: 14 - protein, 239 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSCNVYIMADKQKNGIKVNNKMRHNIEDGSVQLADHYQQNTPIGDGPVLLPDNHYLSAQSALSKDPNEKRDHMVLLEFVTAAGITLGMDELYK

SEQ ID NO: 15 - DNA, 720 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaactgctgtaacgtctatatcatggccgacaagcagaagaacggcatcaaggtgaacaacatgatgcgccacaacatcgaggacggcagcgtgcagctcgccgaccactaccagcagaacacccccatcggcgacggccccgtgctgctgcccgacaaccactacctggctgcacagtccgccctgagcaaagaccccaacgagaagcgcgatcacatggtcctgctggagttcgtgaccgccgccgggatcactctcggcatggacgagctgtacaagtaa

SEQ ID NO: 16 - protein, 239 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNCCNVYIMADKQKNGIKVNNMMRHNIEDGSVQLADHYQQNTPIGDGPVLLPDNHYLAAQSALSKDPNEKRDHMVLLEFVTAAGITLGMDELYK

SEQ ID NO: 17 - DNA, 720 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactcacggcatggacgagctgtacaagtaa

SEQ ID NO: 18 - protein, 239 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITHGMDELYK

SEQ ID NO: 19 - DNA, 720 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactcagggcatggacgagctgtacaagtaa

SEQ ID NO: 20 - protein, 239 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITQGMDELYK

SEQ ID NO: 21 - DNA, 711 bp, artificial sequence
  Codes for LEAT protein
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctgctgtgagcggatgtaccccgaggacggcgccctgaagggcgagatcatgcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 22 - protein, 236 aa, artificial sequence
  LEAT protein
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEACCERMYPEDGALKGEIMQRLKLKDGGHYDAEVKTTYKAKKPVQLPGAYNVNIKLDITSHNEDYTIVEQYERAEGRHSTGGMDELYK

SEQ ID NO: 23 - DNA, 711 bp, artificial sequence
  Codes for LEAT protein
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctgctgtgagcggatgtaccccgaggacggcgccctgaagggcgagatcatgcagaggctgaagctgaaggacggcggccactacgacgctgaggtcaagaccacctacaaggccaagaagcccgtgcagctgcccggcgcctacaacgtcaacgcaaagttggacatcacctcccacaacgaggactacaccatcgtggaacagtacgaacgcgccgagggccgccactccaccggcggcatggacgagctgtacaagtag

SEQ ID NO: 24 - protein, 236 aa, artificial sequence
  LEAT protein
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEACCERMYPEDGALKGEIMQRLKLKDGGHYDAEVKTTYKAKKPVQLPGAYNVNAKLDITSHNEDYTIVEQYERAEGRHSTGGMDELYK

SEQ ID NO: 25 - DNA, 711 bp, artificial sequence
  Codes for LEAT protein
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctgctgtgagcggatgtaccccgaggacggcgccctgaagggcgagaatatgcagaggctgaagctgaaggacggcggccactacgacgctgaggtcaagaccacctacaaggccaagaagcccgtgcagctgcccggcgcctacaacgtcaacgcaaagttggacatcacctcccacaacgaggactacaccatcgtggaacagtacgaacgcgccgagggccgccactccaccggcggcatggacgagctgtacaagtag

SEQ ID NO: 26 - protein, 236 aa, artificial sequence
  LEAT protein
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEACCERMYPEDGALKGENMQRLKLKDGGHYDAEVKTTYKAKKPVQLPGAYNVNAKLDITSHNEDYTIVEQYERAEGRHSTGGMDELYK

SEQ ID NO: 27 - DNA, 717 bp, Aequorea Victoria
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 28 - protein, 238 aa, Aequorea Victoria
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 29 - DNA, 696 bp, Entacmaea quadricolor
atgaattcactgatcaaggaaaatatgcgtatgatggtggtcatggaaggttcggtcaacggctaccaattcaaatgcacaggtgaaggagatggcaatccatacatgggaactcaaaccatgaggatcaaagtcgtcgagggaggacctctgccatttgcctttgacattcttgccacaagcttcatgtatggcagcaagacttttatcaagcacactaaaggcattcctgatttctttaaacagtcctttcctgagggttttacttgggaaagagttacaagatacgaagatggtggagtctttaccgttatgcaggacaccagccttgaagatggctgtctcgtttaccacgccaaagtcactggggtaaactttccctccaatggtgccgtgatgcagaagaagaccaagggttgggagccaaatacagagatgctgtatccagcagatggtggtctgaggggatactctcaaatggcactgaatgttgatggtggtggctatctgtcttgctctttcgaaacaacttacaggtcaaaaaagaccgtcgagaacttcaagatgcccggtttccattttgttgatcaccgcctggaaaggttagaggaaagtgacaaggaaatgttcgtagtacaacacgaacacgcagttgccaagttctgtgaccttccatccaaactgggacgtctttga

SEQ ID NO: 30 - protein, 231 aa, Entacmaea quadricolor
MNSLIKENMRMMVVMEGSVNGYQFKCTGEGDGNPYMGTQTMRIKVVEGGPLPFAFDILATSFMYGSKTFIKHTKGIPDFFKQSFPEGFTWERVTRYEDGGVFTVMQDTSLEDGCLVYHAKVTGVNFPSNGAVMQKKTKGWEPNTEMLYPADGGLRGYSQMALNVDGGGYLSCSFETTYRSKKTVENFKMPGFHFVDHRLERLEESDKEMFVVQHEHAVAKFCDLPSKLGRL

SEQ ID NO: 31 - DNA, 684 bp, Entacmaea quadricolor
atggctggtttgttgaaagaaagtatgcgcatcaagatgtacatggaaggcacggttaatggccattatttcaagtgtgaaggagagggagacggcaacccatttacaggtacgcagagcatgaggattcatgtcaccgaaggggctccattaccatttgccttcgacattttggcaccgtgttgtgagtacggcagcaggacctttgtccaccatacggcagagattcccgatttcttcaagcagtctttccctgaaggctttacttgggaaagaaccacaacctatgaagatggaggcattcttactgctcatcaggacacaagcctggaggggaactgccttatatacaaggtgaaagtccttggtaccaattttcctgctgatggccccgtgatgaagaacaaatcaggaggatgggagccatgcactgaggtggtttatccagagaatggtgtcctgtgtggacgtaatgtgatggcccttaaagtcggtgatcgtcgtttgatctgccatctctatacttcttacaggtccaagaaagcagtccgtgccttgacaatgccaggatttcattttacagacatccgccttcagatgccgaggaaaaagaaagacgagtactttgaactgtacgaagcatctgtggctaggtacagtgatcttcctgaaaaagcaaattga

SEQ ID NO: 32 - protein, 227 aa, Entacmaea quadricolor
MAGLLKESMRIKMYMEGTVNGHYFKCEGEGDGNPFTGTQSMRIHVTEGAPLPFAFDILAPCCEYGSRTFVHHTAEIPDFFKQSFPEGFTWERTTTYEDGGILTAHQDTSLEGNCLIYKVKVLGTNFPADGPVMKNKSGGWEPCTEVVYPENGVLCGRNVMALKVGDRRLICHLYTSYRSKKAVRALTMPGFHFTDIRLQMPRKKKDEYFELYEASVARYSDLPEKAN

SEQ ID NO: 33 - DNA, 684 bp, Echinopora forskaliana
atgagtgtgattaaacaagtaatgaagaccaagttgcacttggagggaactgttaacggacatgactttactattgagggtaaaggagaaggcaaaccttacgaaggattgcagcatatgaaaatgacggtcaccaaaggtgcgcctctgccgttttccgttcacattctgacacctagccatatgtacggaagcaaaccgtttaacaagtatcccgcggacatccctgactaccacaagcagtctttccccgaaggcatgtcttgggagaggagtatgatttttgaagatggaggcgtatgcactgccagcaatcattccagcataaacttgcaagaaaactgtttcatctatgatgtgaaatttcacggcgtgaaccttcctcctgatggtcctgttatgcaaaagaccattgcgggatgggagccgtctgtggagacactgtacgtgcgagacgggatgctgaaaagtgacactgcaatggtttttaagctgaagggaggcggtcatcaccgagttgatttcaagactacttacaaggcaaagaaacctgtcaagttgccagagttccactttgtggagcatcgcctggaactgacaaaacacgacaaggatttcacaacttgggaccagcaggaggcagccgagggtcatttttctccgctgcctaaagctttgccataa

SEQ ID NO: 34 - protein, 227 aa, Echinopora forskaliana
MSVIKQVMKTKLHLEGTVNGHDFTIEGKGEGKPYEGLQHMKMTVTKGAPLPFSVHILTPSHMYGSKPFNKYPADIPDYHKQSFPEGMSWERSMIFEDGGVCTASNHSSINLQENCFIYDVKFHGVNLPPDGPVMQKTIAGWEPSVETLYVRDGMLKSDTAMVFKLKGGGHHRVDFKTTYKAKKPVKLPEFHFVEHRLELTKHDKDFTTWDQQEAAEGHFSPLPKALP

SEQ ID NO: 35 - DNA, 690 bp, Echinopora forskaliana
atgtctttttcaaagcaggttttaaacgacgtgacgatgacctattttatggaaggcagtgtcaacgggcacgactttactattgaaggcgaaggcactggcaaaccatacgaaggacatcaacgttttaatctacgcgtcaccaagggcgcgcctctgcctttcgcagttgacatcctgtcagcagcgtttgcttatggcaaccgatgctttactaagtatcctaaagagataccagactttttcaaacaatcactccccgaagacatgtcatgggagaggacgatgacgttcgaagatggcggtattgttgctatcagtgcgcacatacgccttattggaaaccgcttcgagcacaaatccaagtttgtcggcgtgaacttccccgccgacggacctgtgatgcaaaggaagacgctaggttgggagccttccagcgagaaaatgactccccgcgacggaatactaaagggctatgttccgagtttcctcgtgctgcagggaggtggcaattacagatgcgactacgatactagctacagagccatgaagcctgtggagatgccagggggtcatttcatccagcatcgcattgtcaggagagacatcaagaaagattccaatggcaacacctggcaaattcaagaagacgcgtttgctcataacagcgaggtccctgatagtgcatga

SEQ ID NO: 36 - protein, 229 aa, Echinopora forskaliana
MSFSKQVLNDVTMTYFMEGSVNGHDFTIEGEGTGKPYEGHQRFNLRVTKGAPLPFAVDILSAAFAYGNRCFTKYPKEIPDFFKQSLPEDMSWERTMTFEDGGIVAISAHIRLIGNRFEHKSKFVGVNFPADGPVMQRKTLGWEPSSEKMTPRDGILKGYVPSFLVLQGGGNYRCDYDTSYRAMKPVEMPGGHFIQHRIVRRDIKKDSNGNTWQIQEDAFAHNSEVPDSA

SEQ ID NO: 37 - DNA, 675 bp, Echinopora forskaliana
atgtctcattcaaagcaagctctagccgataccatgaagatgacctggcttatggaaggcagcgtcaatggtcatgcatttaccattgagggagaaggcactggaaaaccttacgagggcaagcaatcagggacattccgtgttacaaagggcggaccccttccatttgcctttgacatagtggcaccgaccttgaagtatggattcaaatgttttatgaagtaccctgctgatatacctgactattttaagctggcatttcccgaaggtcttacatacgacaggaaaatagcatttgaagatggagggtgtgccacggccactgtggaaatgagcctcaaaggcaacactcttgtgcacaagaccaattttcaaggaggcaactttcccattgacgggcctgtcatgcagaaaaggactcttggctgggaaccaacctcagagaaaatgactccttgtgatggaataatcaagggtgacactatcatgtacctgatggttgaaggaggcaaaactctgaaatgccgatatgaaaacaattacagggccaacaagccagttctgatgccaccgagccattttgtggatcttcgccttaccagaaccaaccttgataaggaaggccttgcgtttaaactggaggaatacgctgtggcaagagtcctcgaagtttga

SEQ ID NO: 38 - protein, 224 aa, Echinopora forskaliana
MSHSKQALADTMKMTWLMEGSVNGHAFTIEGEGTGKPYEGKQSGTFRVTKGGPLPFAFDIVAPTLKYGFKCFMKYPADIPDYFKLAFPEGLTYDRKIAFEDGGCATATVEMSLKGNTLVHKTNFQGGNFPIDGPVMQKRTLGWEPTSEKMTPCDGIIKGDTIMYLMVEGGKTLKCRYENNYRANKPVLMPPSHFVDLRLTRTNLDKEGLAFKLEEYAVARVLEV

SEQ ID NO: 39 - DNA, 705 bp, Echinopora forskaliana
atgcagcgtgctgggatgaaggttaaggaacatatgaagatcaaactgcgtatgggaggtactgtaaacggaaagcatttcgcggttaatgggacaggagacggctacccttatcagggaaaacagattttgaaacttatcgtcgaaggcagcgaacctctgcctttcgcttttgatatcttgtcagcagcattccagtatggcaacagggcattcaccgaatacccaacagagatagcagactatttcaagcagtcgtttgagtttggcgaggggttctcctgggaacgaagtttcactttcgaagatggggccatttgcgtcgccaccaacgatataacgatggttggtggtgagtttcagtatgatattcgatttgatggtctgaacttccccgaagatggtccagtgatgcaaaagaaaaccgtaaaatgggagccatccactgagataatgtatatgcaaaatggagtgctgaagggtgaggttaacatggctctgttgcttcaagacaaaagccattaccgttgcgacctcaaaactacttacaaagctaagaataatgtgccgcatcctccaggctaccactatgtggatcactgcattgaaatactcgaagaacgtaaggatcacgttaagctgcgggagcatgctaaagctcgttctagcctgtcacctaccagtgcaaaagaacgaaaggcttag

SEQ ID NO: 40 - protein, 234 aa, Echinopora forskaliana
MQRAGMKVKEHMKIKLRMGGTVNGKHFAVNGTGDGYPYQGKQILKLIVEGSEPLPFAFDILSAAFQYGNRAFTEYPTEIADYFKQSFEFGEGFSWERSFTFEDGAICVATNDITMVGGEFQYDIRFDGLNFPEDGPVMQKKTVKWEPSTEIMYMQNGVLKGEVNMALLLQDKSHYRCDLKTTYKAKNNVPHPPGYHYVDHCIEILEERKDHVKLREHAKARSSLSPTSAKERKA

SEQ ID NO: 41 - DNA, 696 bp, Ricordea florida
atgagtgcactcaaagaggaaatgaaaatcaagcttacattggtgggcgttgttaacgggcacccattcaagatcattggggacggaaaaggcaaaccctatgagggatcgcaggaattaacccttgccgtggtggaaggagggcctctgcctttctcttatgatatcctgacaacgatagttcactatggcaacagggcatttgtgaactacccaaaggacataccagatattttcaagcagacctgctctggtcctggtgctggatattcctggcaaaggaccatgagttttgaagacggaggcgtttgcactgctacgagccatatcagggtggatggcgacactttcaattatgacattcacttcatgggagcggatttccctcttaatggtccagtgatgcagaaaagaacagtgaaatgggagccatccactgagataatgtttcaatgtgatggattgctgaggggtgatgttgccatgtctctgttgctgaaaggaggcggccattaccgatgtgactttaaaactatttataaacccaagaagaatgtcaagatgccaggttaccattttgtggaccactgcattgagataacgagtcaacaggacgattacaacgtggttgagctgtacgagggtgctgtagcccactactctcctctgcagaaaccatgccaagcaaaggcataa

SEQ ID NO: 42 - protein, 231 aa, Ricordea florida
MSALKEEMKIKLTLVGVVNGHPFKIIGDGKGKPYEGSQELTLAVVEGGPLPFSYDILTTIVHYGNRAFVNYPKDIPDIFKQTCSGPGAGYSWQRTMSFEDGGVCTATSHIRVDGDTFNYDIHFMGADFPLNGPVMQKRTVKWEPSTEIMFQCDGLLRGDVAMSLLLKGGGHYRCDFKTIYKPKKNVKMPGYHFVDHCIEITSQQDDYNVVELYEGAVAHYSPLQKPCQAKA

SEQ ID NO: 43 - DNA, 717 bp, Renilla muelleri
atgagtaaacaaatattgaagaacacttgtttacaagaagtaatgtcgtataaagtaaatctggaaggaattgtaaacaaccatgtttttacaatggagggttgcggcaaagggaatattttattcggcaatcaactggttcagattcgtgtcacgaaaggggccccactgccttttgcatttgatattgtgtcaccagcttttcaatatggcaaccgtactttcacgaaatatccgaatgatatatcagattattttatacaatcatttccagcaggatttatgtatgaacgaacattacgttacgaagatggcggacttgttgaaattcgttcagatataaatttaatagaagacaagttcgtctacagagtggaatacaaaggtagtaacttcccagatgatggtcccgtcatgcagaagactatcttaggaatagagccttcatttgaagccatgtacatgaataatggcgtcttggtcggcgaagtaattcttgtctataaactaaactctgggaaatattattcatgtcacatgaaaacattaatgaagtcgaaaggtgtagtaaaggagtttccttcgtatcattttattcaacatcgtttggaaaagacttacgtagaagacggggggttcgttgaacagcatgagactgctattgctcaaatgacatctataggaaaaccactaggatccttacacgaatgggtttaa

SEQ ID NO: 44 - protein, 238 aa, Renilla muelleri
MSKQILKNTCLQEVMSYKVNLEGIVNNHVFTMEGCGKGNILFGNQLVQIRVTKGAPLPFAFDIVSPAFQYGNRTFTKYPNDISDYFIQSFPAGFMYERTLRYEDGGLVEIRSDINLIEDKFVYRVEYKGSNFPDDGPVMQKTILGIEPSFEAMYMNNGVLVGEVILVYKLNSGKYYSCHMKTLMKSKGVVKEFPSYHFIQHRLEKTYVEDGGFVEQHETAIAQMTSIGKPLGSLHEWV

SEQ ID NO: 45 - DNA, 669 bp, Cerianthus sp.
atgaacctgagcaaaaacgtgagcgtgagcgtgtatatgaaggggaacgtcaacaatcatgagtttgagtacgacggggaaggtggtggtgatccttatacaggtaaatattccatgaagatgacgctacgtggtcaaaattgcctacccttttcctatgatatcattaccacggcatttcagtatggtttccgcgtatttacaaaataccctgagggaattgttgactattttaaggattcgcttcccgacgcattccagtggaacagacgaattgtgtttgaagatggtggagtactaaacatgagcagtgatatcacatataaagataatgttctgcatggtgacgtctgggctgtcggagtgaacttcccgccgaatgggccagtgatgaagaatgaaattgtgatggaggaaccgactgaagaaacatttactccaaaaaacggggttcttgttggcttttgtcccaaagcgtacttacttaaagatggttcctattactatggaaatatgacaacattttacagatccaagaaatctggccaggcacctcctgggtatcactttgttaagcatcgtctcgtcaagaccaatgtgggacatggatttaagacggttgagcagactgaatatgccactgctcatgtcagtgatcttcccaagtaa

SEQ ID NO: 46 - protein, 222 aa, Cerianthus sp.
MNLSKNVSVSVYMKGNVNNHEFEYDGEGGGDPYTGKYSMKMTLRGQNCLPFSYDIITTAFQYGFRVFTKYPEGIVDYFKDSLPDAFQWNRRIVFEDGGVLNMSSDITYKDNVLHGDVWAVGVNFPPNGPVMKNEIVMEEPTEETFTPKNGVLVGFCPKAYLLKDGSYYYGNMTTFYRSKKSGQAPPGYHFVKHRLVKTNVGHGFKTVEQTEYATAHVSDLPK

SEQ ID NO: 47 - DNA, 699 bp, Anthomedusae sp.
atggaaggtggtccagcattatttcaatccgatatgacattcaagatcttcatcgatggagtggtgaatgatcagaaattcacgataatcgcagatggatcgtccaaattcccccatggtgacttcaacgtgcatgctgtgtgcgaaaccgggaaactcccaatgtcatggaaacctatttgtcaccttatccaatacggggagccattctttgcaaaatatcccaatggcatcagccattttgcacaggagtgctttccagaaggattaacaattgatcgaacagtcagattcgaaaatgacggcactatgacgtctcaccacacctatgagttggacggcacctgtgtcatttccaggataaccgttaattgtgacggatttcaacctgatggaccaatcatgaaagaccagcttgttgatatcctgccaactgagacacatatgttccctcatgggtccaatgctgtcagacaattgtgctacattggcttcacgacagctgatggtggtctcatgatgtcacattttgattcgaaattgacattcaatggttcgagagcaatcaagattcctggacctcatttcgttactgtgataatcaaacagatgaaagatacaagcgacaagcgtgatcatgtgtgtcaacgtgaagtcacctacgctcactcagttccacgcatcacttctgctatctaa

SEQ ID NO: 48 - protein, 232 aa, Anthomedusae sp.
MEGGPALFQSDMTFKIFIDGVVNDQKFTIIADGSSKFPHGDFNVHAVCETGKLPMSWKPICHLIQYGEPFFAKYPNGISHFAQECFPEGLTIDRTVRFENDGTMTSHHTYELDGTCVISRITVNCDGFQPDGPIMKDQLVDILPTETHMFPHGSNAVRQLCYIGFTTADGGLMMSHFDSKLTFNGSRAIKIPGPHFVTVIIKQMKDTSDKRDHVCQREVTYAHSVPRITSAI

SEQ ID NO: 49 - DNA, 705 bp, Aequorea Victoria
atgtctagtggagcactgttgttccacggaaagatcccatatgttgttgagatggagggaaatgttgatggacacacattctccattagaggtaaaggttatggagatgcaagtgttggtaaagttgatgcccaattcatctgcacaactggagatgtaccagttccatggtcaactttagtaacaacacttacttatggtgcacaatgcttcgccaaatatggtccagaattaaaggatttctacaagagttgcatgcctgaaggctatgtgcaggagcgtacaatcacatttgaaggggacggagtatttaaaactcgcgctgaagttacatttgaaaacggatctgtttataaccgagtcaaacttaatggacaaggatttaagaaagacggacatgtgcttggaaagaatcttgaattcaatttcacacctcattgtctttacatttggggagatcaggctaatcatggtttgaagtctgctttcaaaattatgcatgagattactggatcaaaagaagacttcattgttgcagaccacacccaaatgaacacacccattggtggtggaccagtccatgtccctgaataccatcatataacataccatgtcactctcagcaaagatgttactgatcacagggataacatgagcttggttgaaaccgtacgggctgtggattgcagaaaaacatatctttaa

SEQ ID NO: 50 - protein, 234 aa, Aequorea Victoria
MSSGALLFHGKIPYVVEMEGNVDGHTFSIRGKGYGDASVGKVDAQFICTTGDVPVPWSTLVTTLTYGAQCFAKYGPELKDFYKSCMPEGYVQERTITFEGDGVFKTRAEVTFENGSVYNRVKLNGQGFKKDGHVLGKNLEFNFTPHCLYIWGDQANHGLKSAFKIMHEITGSKEDFIVADHTQMNTPIGGGPVHVPEYHHITYHVTLSKDVTDHRDNMSLVETVRAVDCRKTYL

SEQ ID NO: 51 - DNA, 660 bp, Chiridius poppei
atgacaaccttcaaaatcgagtcccggatccatggcaacctcaacggggagaagttcgagttggttggaggtggagtaggtgaggagggtcgcctcgagattgagatgaagactaaagataaaccactggcattctctcccttcctgctgtcccactgcatgggttacgggttctaccacttcgccagcttcccaaaggggactaagaacatctatcttcatgctgcaacaaacggaggttacaccaacaccaggaaggagatctatgaagacggcggcatcttggaggtcaacttccgttacacttacgagttcaacaagatcatcggtgacgtcgagtgcattggacatggattcccaagtcagagtccgatcttcaaggacacgatcgtgaagtcgtgtcccacggtggacctgatgttgccgatgtccgggaacatcatcgccagctcctacgctagagccttccaactgaaggacggctctttctacacggcagaagtcaagaacaacatagacttcaagaatccaatccacgagtccttctcgaagtcggggcccatgttcacccacagacgtgtcgaggagactcacaccaaggagaaccttgccatggtggagtaccagcaggttttcaacagcgccccaagagacatgtag

SEQ ID NO: 52 - protein, 219 aa, Chiridius poppei
MTTFKIESRIHGNLNGEKFELVGGGVGEEGRLEIEMKTKDKPLAFSPFLLSHCMGYGFYHFASFPKGTKNIYLHAATNGGYTNTRKEIYEDGGILEVNFRYTYEFNKIIGDVECIGHGFPSQSPIFKDTIVKSCPTVDLMLPMSGNIIASSYARAFQLKDGSFYTAEVKNNIDFKNPIHESFSKSGPMFTHRRVEETHTKENLAMVEYQQVFNSAPRDM

SEQ ID NO: 53 - DNA, 30 bp, Forward primer
cccaagcttatggtgagcaagggcgaggag

SEQ ID NO: 54 - DNA, 31 bp, Reverse primer
ccgtctagactacttgtacagctcgtccatg

SEQ ID NO: 55 - DNA, 30 bp, Forward primer; identical to SEQ ID NO: 53

SEQ ID NO: 56 - DNA, 31 bp, Reverse primer
ccgtctagattacttgtacagctcgtccatg

SEQ ID NO: 57 - DNA, 28 bp, Forward primer
cccaagcttatgagtaaaggagaagaac

SEQ ID NO: 58 - DNA, 30 bp, Reverse primer
ccgtctagattatttgtatagttcatccat

SEQ ID NO: 59 - DNA, 30 bp, Forward primer
cccggatccatggtgagcaagggcgaggag

SEQ ID NO: 60 - DNA, 31 bp, Reverse primer
ccggtcgacctacttgtacagctcgtccatg

SEQ ID NO: 61 - DNA, 696 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcacttaa

SEQ ID NO: 62 - protein, 231 aa, artificial sequence
  LEAT protein
MVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGIT

SEQ ID NO: 63 - DNA, 696 bp, artificial sequence
  Codes for LEAT protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcacttaa

SEQ ID NO: 64 - protein, 231 aa, artificial sequence
  LEAT protein
MVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGIT